(19) United States
(12) Patent Application Publication
(10) Pub. No.: US 2003/0023034 A1
(43) Pub. Date: Jan. 30, 2003

(54) p27 (Kip1) -FKBP-12 protein complexes

(76) Inventors: Krishnan Nandabalan, Guilford, CT (US); Meijia Yang, East Lyme, CT (US)

Correspondence Address: MINTZ, LEVIN, COHN, FERRIS,, GLOVSKY AND POPEO, P.C., One Financial Center, Boston, MA, 02111, US

(21) Appl. No.: 09/970,561

(22) Filed: Oct. 3, 2001

Related U.S. Application Data
(63) Continuation of application No. 09/099,857, filed on Jun. 18, 1998, now abandoned.

(51) Int. Cl. G01N 33/53; C07K 14/435; C07K 16/40

(52) U.S. Cl. 530/350; 530/388.1; 435/7.1

(57) ABSTRACT

The present invention is directed to complexes of the protein p27(Kip1) with proteins identified as interacting with p27(Kip1) by a yeast mating test. Specifically, the present invention is directed to complexes with p27(Kip1) and FKBP-12. The present invention is also directed to complexes of a derivative, fragment or analog of p27(Kip1) and a derivative, fragment or analog of FKBP-12. Methods of screening the complexes for efficacy in treating and/or preventing certain diseases and disorders, particularly hyperproliferative disorders, including cancer, neurodegenerative disease, autoimmune disease, are also provided.

This invention was made with United States Government support under grant number 70NANB5 H1066 awarded by the National Institute of Standards and Technology. The United States Government has certain rights in the invention.

### 1. FIELD OF THE INVENTION

The present invention is directed to complexes of p27(Kip1) and FKBP-12 proteins. The invention is also directed to antibodies specific for such complexes and to methods of using such antibodies.

### 2. BACKGROUND OF THE INVENTION

2.1. p27(Kip1)

2.1.1. Role of p27(Kip1) in the Eukaryotic Cell Cycle

Eukaryotic cell cycle progression is controlled by the activation and inactivation of a highly conserved family of protein complexes. The protein complex minimally consists of a catalytic subunit having kinase activity (cyclin-dependent kinase or CDK) and a regulatory subunit (cyclin). Each phase of the cell cycle is characterized by the expression of a unique profile of such cyclin-CDK complexes. For instance, the commitment of cells to enter the DNA synthesis (S) phase of the cell cycle occurs at a restriction (R) point late in the first gap (G1) phase of the cell cycle. Progression through this first gap phase is regulated by the activity of the D-type cyclins and cyclin E, which associate with CDK4 and CDK6, respectively. Sherr & Roberts, 1995, Genes and Dev. 9:1149-1163.

CDK inhibitors are negative regulatory proteins that bind to a cyclin-CDK complex and inhibit their catalytic activity. Sherr & Roberts, 1995, Genes and Dev. 9:1149-1163. Two families of low molecular weight proteins which are CDK inhibitors are the Ink4 family and the Cip1/Kip1 family of proteins. The Cip1/Kip1 family of proteins, which includes p21(Cipl), p27(Kip1)(Kip1), and p57(Kip2), have broad specificity and potently inhibit the activity of most cyclin-CDK complexes. Cip1/Kip1 proteins appear to regulate cell proliferation by stopping cell cycle progression in response to a variety of anti-mitogenic signals. The importance of these cyclin inhibitor proteins is evidenced by the fact that cancer development and/or progression is strictly linked to alterations of molecular mechanisms controlling the cell division cycle.

Human p27(Kip1) (27 kDa kinase inhibitor protein), encodes a 22 kDa protein of 198 amino acids and is broadly expressed in human tissues with similar p27(Kip1) encoding mRNA levels in both proliferating and quiescent cells. Polyak et al., 1994, Cell 78:59-66; Toyoshima and Hunter, 1994, Cell 78:67-74; U.S. Pat. No. 5,688,665. The nucleotide sequence is available in GenBank under Accession No. U10906. p27(Kip1) appears to be primarily responsible for regulating CDK activity by inhibiting cyclin-CDK complex-associated kinase activity (for example, cyclin D-CDK4, cyclin E-CDK2, and cyclin A-CDK2) in response to extracellular antiproliferative cues, thereby arresting the cell-cycle and preventing proliferation. Nomura et al., 1997 Gene 191:211-218. The involvement of p27(Kip1) in the negative regulation of cell proliferation suggests that it also functions as a tumor suppressor gene. In addition to 25 its role as an inhibitor, p27(Kip1) may function as an adaptor protein to facilitate assembly of specific CDK/cyclin complexes which will have specific functions. LaBaer et al., 1997, Genes and Dev. 11:847-862.

2.1.2. Functional Domains of p27(Kip1)

Several functional domains of p27(Kip1) have been identified. One such domain is an amino-terminal domain of 60 amino acids that is both necessary and sufficient for cyclin/CDK complex binding and inhibition. Within this domain, a minimal inhibitory region responsible for inhibiting cyclin-CDK associated kinase activity has been determined to comprise amino acid residues 28-79. Polyak et al., 1994, Cell 78:59-66; Toyoshima and Hunter, 1994, Cell 78:67-74; Kwon et al., 1996, Biochem. Biophys. Res. Comm. 220:703-709.

Another domain at the amino-terminal region of p27(Kip1) is a cyclin binding motif. Cyclin binding motifs are also found in other CDK inhibitors and is also found in other proteins, including retinoblastoma gene family members p207 and pl30, and transcription factors E2F-1, E2F-2, and E2F-3.

The carboxyl-terminal domain of p27(Kip1) can bind cyclin Dl in vitro, suggesting that p27(Kip1) associates with D-type cyclins independently of CDK4. Further, is has been demonstrated that CDC2 kinase activity is down-regulated by the carboxyl-terminal region of p27(Kip1). Also, amino acid residue Thr187 located in the carboxyl-terminal domain of p27(Kip1) is a potential substrate site for cyclin-CDK phosphorylation. Vlach et al., 1997, EMBO J. 16:5334-5344.

It has been demonstrated that the oncogenic adenovirus protein E1A binds to p27(Kip1), however, the interacting domain in p27(Kip1) has not been identified. Mal 1996, Nature 380: 262-265.

2.1.3. Regulation of p27(Kip1) Expression

In normal cells, expression of p27(Kip1) increases during entry into a quiescent or nondividing state, and rapidly decreases upon re-entry into the cell cycle after stimulation with specific growth factor(s). The abundance of the p27(Kip1) protein is believed to be regulated mainly by translational and post-translational control mechanisms, although some regulation is seen on the transcriptional level.

2.1.3.1. Transcriptional Regulation

Transcriptional regulation of the p27(Kip1) gene may be involved in cellular differentiation. This is indicated by the fact that p27(Kip1) mRNA is downregulated by 1,25-dihydroxyvitamin D3 (Vitamin D3). Vitamin D3 acts through its cognate nuclear receptor (Vitamin D3 receptor) to induce a myeloid leukemic cell line to terminally differentiate into monocytes/macrophages; overexpression of p27(Kip1) directly leads to a terminal differentiation program in these myeloid cells. Liu, 1996, Genes Dev 10:142-153; Wang et al., 1997, Cancer Res 57:2851-2855. Furthermore, the mitogen Interleukin-2 also influences the function of the p27(Kip1) gene promoter, which effectively results in the transcriptional down-regulation of p27(Kip1), which results in a significant reduction of p27(Kip1), and thus, the cells are stimulated to progress through the cell cycle. Kwon et al., 1997, J. Immunol. 158:5642-5648. Nevertheless, mRNA levels of p27(Kip1) are relatively constant in pituitary adenomas and carcinomas, suggesting that p27(Kip1) protein levels are mainly regulated by translational and post-translational mechanisms.

2.1.3.2. Translational and Post-Translational Regulation

p27(Kip1) is expressed at high levels in hepatoma cells, macrophages, fibroblasts, T-lymphocytes, astroglial cells, and mesanglial cells, and this high level of expression is decreased in response to insulin (Mann et al., 1997, Oncogene 14:1759-1766), colony-stimulating factor 1 25 (Antonov et al., 1997, J. Clin. Invest. 99:2867-2876), serum (Dietrich et al., 1997 Oncogene 15:2743-2747), Interleukin-2 (IL-2) (Dumont, 1996, Life Sci. 58:373-395), Interleukin-4 (IL-4) (Liu et al., 1997, J. Imm. 159:812-819), and platelet-derived growth factor (PDGF) (Shankland, 1997, Kidney Int. 51:1088-1099).

Other events that regulate p27(Kip1) expression include mitogen deprivation, cell-cell contact, and addition of transforming growth factor (TGF)-β or p53 to a p27(Kip1) expressing cell. The down-regulation of p27(Kip1) by mitogens occurs mainly through the ubiquitin-dependent post-translational degradation pathway. Pagano et al., 1995, Science 269:682-685; Esposito et al., 1997 Cancer Res. 57:3381-3385. Also, the phosphorylation of p27(Kip1) by CDK2 at the carboxyl-terminal CDK target site of amino acid residues 187-190 (TPKK) is essential for this post-translational degradation.

When T-cells are stimulated with IL-2, cyclin E-CDK2 complexes become activated and phosphorylate p27(Kip1). The phosphorylated p27(Kip1) is eliminated via the ubiquitin-dependent pathway and the cell cycle progresses. This activation of the cell cycle by degradation of p27(Kip1) can be prevented by the immunosuppressant drug rapamycin, which inhibits the enzymatic activity of cyclin E-CDK2 and results in the presence of abnormally high levels of p27(Kip1) in rapamycin treated cells. Consistent with this mechanism, fibroblasts and T-lymphocytes with a targeted disruption of the p27(Kip1) gene display impaired growth-inhibitory responses to rapamycin. Luo et al., 1996, Mol. Cell. Biol. 16:6744-6751. The antiproliferative effect of rapamycin is mediated indirectly and only after binding of rapamycin to its receptor FKBP-12 (see Section 2.2, infra).

Similarly to regulation of p27(Kip1) by rapamycin, transforming growth factor-β (TGF-β) can control the cell cycle by regulating normal and neoplastic cell function and by regulating the expression of various proteins, including p27(Kip1). Jin, 1997, Am. J. Path. 151:509-519; Polyak et al., 1994, Genes Dev 8:9-22. TGF-β disrupts the signaling pathway that coordinates the GI to S phase transition in the cell cycle through several mechanisms, including the upregulation of p27(Kip1) which results in the inhibition of activation of cyclin D-CDK4 complex activity, and inhibition of cyclin E-CDK2 complex activity resulting in the hypophosphorylation of Rb protein. TGF-β inhibition can be reversed by the oncogenic adenovirus protein E1A, which binds to and thereby inhibits the activity of p27(Kip1), such that cells can proceed though the cell cycle. Mal et al., 1996, Nature 380:262-265; Nomura et al., 1997, Gene 1991:211-218; Carneiro et al., 1998, Oncogene 16(11):1455-1465; Muller et al., 1997, Oncogene 15(21):2561-2576.

Hengst and Reed, 1996, Science 271:1861-1864 showed that p27(Kip1) protein levels vary throughout the cell cycle; however, the levels of p27(Kip1)-encoding messenger RNA remained constant throughout the cell cycle, and concluded that translational control of p27(Kip1) is an important mechanism for controlling p27(Kip1) protein levels.

In summary, transcriptional, translational and post-translational regulation of p27(Kip1) provide a critical mechanistic link between mitogenic signals and cell cycle progression.

2.1.4.. Role of p27(Kip1) in Tumorigenesis and Tumor Suppression

Through their roles in cell cycle control, CDK inhibitors such as p27(Kip1) play significant roles in various biological phenomena such as cancer development and/or progression, neuronal differentiation, and apoptosis. Cancer development and/or progression is strictly linked to alterations of molecular mechanisms controlling the cell division cycle. p27(Kip1) regulates the progression through the G1 phase of the cell cycle and at the G1/S phase transition. Thus, p27(Kip1) is implicated in numerous cancers, including leukemia, lymphoma, breast cancer, pancreatic cancer, colorectal cancer, and lung cancer.

Mice lacking p27(Kip1), referred to as “p27(Kip1)(−/−)” herein, are larger than control animals, with thymus, pituitary and adrenal glands, and gonadal organs exhibiting striking enlargement. This is the result of increased numbers of cells in all tissues and organs and confirms the importance of p27(Kip1) in the control of cell proliferation. Similar to mice with a retinoblastoma (Rb) gene mutation, the p27(Kip1)(−/−) mice often develop pituitary tumors spontaneously. This clearly shows that p27(Kip1) plays an important role in inhibiting tumor formation and that p27(Kip1) may act as a growth regulator of a variety of cells.

Despite tumor development in p27(Kip1)(−/−) mice, the p27(Kip1) gene has never been observed to be inactivated in human tumors, and mutations in p27(Kip1) have been detected only in rare cases of primary adult T cell leukemia, non-Hodgkin lymphoma and human breast carcinoma. Hatta et al., 1997, Leukemia 11:984-989; Ferrando, 1996, Human Genetics, 97:91-94. Moreover, reduced levels of p27(Kip1) predict poor survival of patients with breast, colorectal and pancreatic cancer. Fredersdorf et al., 1997, Proc. Nat. Acad. Sci. USA 94:6380-6385; Groshong et al., 1997, Mol. Endocrinol. 11:1593-1607; Yasui et al., 1997, Japanese J. Cancer Res. 88:625-629; Kawa et al., 1997, Int. J. Cancer 72:906-911. p27(Kip1) expression levels correlate with cancer progression since a decrease in p27(Kip1) expression levels significantly correlates with advanced stage, depth of tumor invasion and lymph node metastasis. Thus, p27(Kip1) is directly implicated in human cancer and expression levels of p27(Kip1) can serve as a useful prognostic marker in cancer.

2.1.5. Role of p27(Kip1) in Differentiation

p27(Kip1) has been observed to be involved in the differentiation of a number of cell types. For example, the introduction of p27(Kip1) into neuronal, hematopoietic, and muscle precursor cells accelerates their differentiation. Kranenburg et al., 1995, J. Chem. Biol. 87:1225-1235; Liu et al., 1996, Genes and Dev. 10:142-153; Guo et al., 1995, Mol. Cell. Biol. 15:3823-3829. Further, p27(Kip1) was found to be down-regulated in a subset of developing thymocytes (Hoffman et al., 1996 Genes and Dev. 9:948-962) and high levels of p27(Kip1) accumulate in cortical post-mitotic neurons during mouse neurogenesis whereas low levels were found in-their progenitor neuroblasts. Also, elevated levels of p27(Kip1) in staged embryo brain extracts correlate with binding of p27(Kip1) to CDK2. Lee et al., 1996, Proc. Nat. Acad. Sci. USA 93:3259-3263. p27(Kip1) mediates the withdrawal of oligodendrocyte progenitor cells (0-2A) from the cell cycle during development of the central nervous system, and accumulation of p27(Kip1) in these progenitor cells correlates with differentiation of oligodendrocytes. Casaccia-Bonnefil et al., 1997, Genes and Dev. 11:2335-2346.

2.1.6. Role pf p27(KiP1) in Apoptosis

During hormone-induced apoptosis, expression of p27(Kip1) increases, and as a result, the G2/M phase transition of the cell cycle is blocked. Furuya et al., 1997, Anticancer Res. 17:2089-2093. Similarly, growth arrest in anti-IgM induced B-cell lymphomas is dependent on increased synthesis of p27(Kip1). Generally, increased levels of p27(Kip1) correlate with reduced phosphorylation of the retinoblastoma gene product, which leads to cell cycle arrest and subsequent apoptosis. Scott et al., 1997, Curr. Top. Microbiol. Immunol. 224:103-112; Eyhevesky et al., 1996, Mol. Biol. Cell. 7:553-564. This is in contrast to that seen in tumor proliferation where p27(Kip1) levels are significantly decreased.

2.1.7. Role of p27(Kip1) in Atherosclerosis

During atherosclerosis and re-stenosis, abnormal proliferation of vascular smooth muscle cells (VSMC) contribute to intimal hyperplasia. The downregulation of CDK2 activity in these cells is mediated by CDK2-p27(Kip1) complexes. Chen et al., 1997, J. Clin. Invest. 99:2334-2341. Further, accumulation of monocyte-derived macrophages, which accumulation also contributes to plaque formation, is driven by macrophage colony stimulating factor (MCSF) present in atherosclerotic plaques. Interestingly, MCSF is required for successful down-regulation of p27(Kip1) before cell cycling. Antonov et al., 1997, J. Clin. Invest. 99:2867-2876.

2.1.8. Role of p27(Kip1) in Membranous Nephropathy

In progressive glomerulonephritis, the thickening of glomerular mesangial cells is associated with a marked up-regulation in expression of cyclin kinase inhibitors p27(Kip1) and p21(Cipl). Shankland et al., 1997, Kidney Int. 52:404-413. Such up-regulation of expression of these kinase inhibitors leads to cell cycle arrest and results in decreased cell proliferation, reduced glomerular function, and resultant renal insufficiency.

In summary, p27(Kip1) is implicated in the control of cell cycle progression, and thus, has a role in tumorigenesis, tumor progression and spread, neuronal differentiation, apoptosis, atherosclerosis, and nephropathy.

2.2. FKBP-12

The low molecular weight (11.8 kDa) cytosolic drug-binding protein FKBP-12 catalyzes the slow cis to trans isomerization of a Xaa-proline peptide bond in short synthetic peptides. Siehkierka et al., 1989, Nature 341:755-757; Galat, 1993, Eur. J. Biochem. 216:689-707. It has thus been classified as a peptidyl-prolyl-cis-trans isomerase (PPIase). Other proteins in this class are the cyclosporin A-binding proteins or cyclophilins. The similar enzymatic activity of FKBP-12 and cyclophilins, together with their ability to serve as receptors for immunosuppressive agents, has justified the generic denomination of “immunophilins” for these proteins. Marks, 1996, Phys. Reviews 76:631-649.

FKBP-12 binds to the immunosuppressant FK506, hence the name FK506-binding protein. Also, FKBP-12 selectively binds with equivalent affinity to another potent and clinically useful immunosuppressant, rapamycin, and probably mediates rapamycin-dependent immunosuppression. Both FK506 and rapamycin have realized or potential clinical applications in the prevention of graft rejection after organ transplantation and the treatment of autoimmune disorders.

FKBP-12 has been isolated from calf thymus, human spleen (Harding et al., 1989, Nature 341:761-763) and T-lymphoma cells (Siekierka et al., 1989, Nature 341:755-757) and was first cloned by Standaert et al., 1990, Nature 346:641-674 and Maki et al., 1990, Proc. Nat. Acad. Sci. USA 87:5440-5443. The nucleotide sequence of FKBP-12 is available in GenBank under Accession No. X55741. Human FKBP-12 is a 108 amino acid protein and its encoding gene resides on chromosome 20. DiLella, 1991, Biophys. Biochem. Res. Comm. 179:1427-1433. FKBP-12 genes are highly conserved over evolution; the genes from human, mouse, rat and bovine cells have a high sequence homology (97%). Three distinct transcripts encode human FKBP-12. The transcripts contain identical open reading frames but vary in abundance and are distinguished by unique 3′ untranslated regions. The mature transcripts derive from five exons of the genomic human FKBP-12 gene sequence (24 kb). Peattie, 1994, Gene 150:251-257. The exon modules correlate with structural features, namely separate exons encode the anti-parallel beta strands and an alpha helix. DiLella and Craig, 1991, Biochemistry 30:8512-8517.

X-ray crystallography studies have revealed that FKBP-12 has a compact globular structure, containing five anti-parallel beta-sheets which wrap around a short alpha-helix, amino acid residues 58-64. The immunosuppressant drugs rapamycin or FK506 bind in an oval-shaped deep hydrophobic pocket (containing Tyr26, Phe46, Phe99, Val55, Ile56, Trp59) between beta sheets 3 and 4 and the helix and make contact with the protein through hydrophobic interactions and intermolecular hydrogen bonds, thereby forming a unique effector molecular complex. Whereas the FKBP-12:FK506 complex interacts with and inhibits a Ca2+-dependent serine-threonine phosphatase (calcineurin), the FKBP-12:rapamycin complex affects unique biochemical processes of cytokine-mediated signal transduction and blocks the transition from GI to S phase in the cell cycle.

FKBP-12 is also critical to intracellular Ca²⁺ regulation through effects on the ryanodine and inositol-triphosphate receptors that control calcium-efflux from the sarcoplasmic and endoplasmic reticulum. FKBP-12 is physically associated with and modulates the function of the major Ca²⁺ release channel/ryanodine receptor of the sarcoplasmic reticulum of skeletal and cardiac muscles. The FKBP-12:FK506 complex specifically binds to and inhibits calcineurin, a Ca²+and calmodulin binding signaling protein required for transcriptional activation of the interleukin-2 gene in response to T-cell antigen receptor engagement. Abraham & Wiederrecht, 1996, Ann. Rev. 1 mm. 14:483-510. FKBP-12 was also found to be an integral component of the intracellular calcium-release channel complex and can modulate the function of these channels by effecting the channel gating. Brillantes et al., 1994, Cell 77: 513-323.

FKBP-12 also interacts with the type I receptor for transforming growth factor-β (TGF-β RI) and inhibits its signaling function. Wang et al., 1996, Cell 86:435-444. FKBP-12 binding to TGF-β receptor involves the rapamycin/Leu-Pro binding pocket of FKBP-12 and a Leu-Pro sequence located next to the activating phosphorylation sites in the TGF-β receptor I. This interaction is competitively inhibited by excess FK506; similarly, rapamycin competes with the binding of FKBP-12 to TGF-β receptor. Chen et al., 1997, EMBO J 16:3866-3876. It is believed that FKBP-12 binding is inhibitory to the signaling pathways of the TGF-β family ligands.

Finally, a distinct function of rapamycin is the involvement of FKBP-12 in ligand-activated immunosuppression and inhibition of cellular proliferation. During the binding of rapamycin to its cytosolic receptor FKBP-12, several biochemical alterations in the cell are mediated. The potent antiproliferative activity of rapamycin involves binding to FKBP-12, and subsequent interaction with targets of rapamycin, resulting in the inhibition of p70S6 kinase. However, neither p70S6 kinase inhibition, nor p27(Kip1)-induced cyclin E-CDK2 inhibition are directly mediated by the FKBP-rapamycin complex. Instead this complex physically interacts with the mTOR protein which has sequence homology with the catalytic domain of phosphatidylinositol kinases. Dumont and Su, 1996, Life Sci. 58:373-395. According to the linear pathway hypothesis, mTOR affects p27(Kip1) levels and G1 phase CDKs by modulating the activity of p70S6 kinase on protein synthesis or certain transcriptional events.

In summary, FKBP-12 is implicated in the control of cell cycle progression in various biological phenomena such as tumorigenesis, and tumor progression and spread. Furthermore, FKBP-12 is involved in immunosuppression and may have significant roles in organ transplantation and autoimmune diseases. Also, FKBP-12 is involved in the regulation of calcium-efflux in cardiac and skeletal muscles. In addition, FKBP-12 plays a role in cytokine-mediated signal transduction.

p27(Kip1) and FKBP-12 have been described to be involved in similar processes, however no direct association or interaction of p27(Kip1) with FKBP-12 has been described previously to the present invention.

Citation or identification of any reference in Section 2 or any other section of this application shall not be construed as an admission that such reference is available as prior art to the present invention.

### 3. SUMMARY OF THE INVENTION

The present invention is based, in part, upon the inventors' discovery that p27(Kip1) binds to and forms a complex with a p27(Kip1) binding protein, such as FKBP-12. Accordingly, the present invention is directed to compositions and methods of production of protein complexes of p27(Kip1) with a protein that interacts with, i.e., binds to, p27(Kip1). Specifically, the invention is directed to a complex of p27(Kip1), or a derivative, fragment or analog thereof, with FKBP-12, or a derivative, analog or fragment thereof. A complex of p27(Kip1) and FKBP-12 is designated as “p27(Kip1):FKBP-12” herein. The present invention is further directed to methods of screening for proteins that interact with p27(Kip1), or that interact with a derivative, fragment or analog of p27(Kip1). Preferably, the method for screening is a matrix mating test or a variation thereof, see Section 6.

The present invention further provides methods for production of a p27(Kip1):FKBP-12 complex, or a derivative or analog of the complex and/or individual protein by, e.g., recombinant means. Pharmaceutical compositions are also provided in the present invention.

The present invention is also directed to methods for modulating, i.e., inhibiting or enhancing, the activity of a p27(Kip1):FKBP-12 complex or formation of said complex. The protein components of the p27(Kip1):FKBP-12 complex have been implicated in a variety of cellular functions, including, but are not limited to, physiological processes such as control of cell cycle progression, cellular differentiation and apoptosis, intracellular signal transduction, neurogenesis, response to viral infection, and pathophysiological processes, which include hyperproliferative disorders such as tumorigenesis and tumor spread, degenerative disorders such as neurodegenerative disease and autoimmune disease, disorders associated with organ transplantation, inflammatory and allergic disease, atherosclerosis, nephropathy and cardiac and muscle disease.

Accordingly, the present invention also provides for methods for screening a p27(Kip1):FKBP-12 complex, as well as screening for a derivative or analog of the p27(Kip1):FKBP-12 complex for the ability to alter cell function, particularly a cell function in which p27(Kip1) and/or FKBP-12 has been implicated.

The present invention is also directed to therapeutic, prophylactic, diagnostic, prognostic, as well as screening methods and compositions for use in such methods based on a p27(Kip1):FKBP-12 complex, and nucleic acid molecules encoding the individual proteins that participate in such a complex. Therapeutic compounds of the invention include, but are not limited to, a p27(Kip1):FKBP-12 complex or a complex where one or both members of the complex is a derivative, homolog, or analog of p27(Kip1) or FKBP-12; antibodies specific for and nucleic acid molecules encoding the foregoing p27(Kip1) or FKBP-12 proteins; and antisense nucleic acids complementary to the nucleotide sequences encoding the complex components. The present invention also provides kits for diagnostic, prognostic and screening use.

Animal models and methods of screening for modulators, i.e., agonists, antagonists, of p27(Kip1):FKBP-12 complex activity are also provided.

Methods of identifying molecules that inhibit, or alternatively, that increase formation of a p27(Kip1):FKBP-12 complex are also provided in the present invention.

### 4. BRIEF DESCRIPTION OF TRE DRAWINGS

FIG. 1. The nucleotide sequence (GenBank Accession No. U10906) (SEQ ID NO: 1) and deduced amino acid sequence (SEQ ID NO:2) of p27(Kip1). The coding sequence beginning at base 127 (amino acid 43), indicated by an arrow, and ending at base 597 (amino acid 198) was used as bait in the assays described in Section 6, infra.

FIG. 2. The nucleotide sequence (GenBank Accession No. X55741) (SEQ ID NO:3) and deduced amino acid sequence (SEQ ID NO:4) of human FKBP-12. The 5′ start site of the identified prey sequence was at base 109 (amino acid 34), indicated by an arrow.

FIG. 3. Demonstration of the specificity of p27(Kip1):FKBP-12 interaction. The results of the matrix mating test using p27(Kip1) and proteins A1 and B1 as bait are indicated above the columns and the prey proteins CDK2 (positive control), FKBP-12, TrkA, CYC-B (cyclophilin B), and Vector (vector control; negative control) are indicated to the left of the rows. A positive interaction between bait and prey proteins is indicated as ‘+’, a lack of interaction is designated by ‘−’.

### 5. DETAILED DESCRIPTION OF THE INVENTION

The present invention is based, in part, upon identification of proteins that interact with p27(Kip1) using a modified form of the yeast matrix mating test. FKBP-12 was found to form a complex under physiological conditions with p27(Kip1) (the complex of p27(Kip1) with FKBP-12 is indicated as “p27(Kip1):FKBP-12” herein). The p27(Kip1):FKBP-12 complex, by virtue of the interaction, is implicated in modulating the functional activities of p27(Kip1) and its binding partner. Such functional activities include, but are not limited to, physiological processes such as control of cell cycle progression, cellular differentiation and apoptosis, intracellular signal transduction, neurogenesis, response to viral infection, and pathophysiological processes including hyperproliferative disorders such as tumorigenesis and tumor spread, degenerative disorders such as neurodegenerative diseases, autoimmune disease, disorders associated with organ transplantation, inflammatory and allergic disease, atherosclerosis, nephropathy and cardiac and muscle diseases.

The present invention is also directed to methods of screening for proteins that interact with, e.g., bind to p27(Kip1). The present invention is further directed to p27(Kip1) complexes, in particular p27(Kip1) complexes with FKBP-12. The present invention is also directed to a complex of p27(Kip1) or a derivative, analog or fragment of p27(Kip1) with a derivative, analog or fragment of FKBP-12. In a preferred embodiment, such complexes bind an anti-p27(Kip1):FKBP-12 complex antibody. In a specific embodiment of the present invention, a complex of human p27(Kip1) with human FKBP-12 is provided.

The present invention also provides methods of producing and/or isolating a p27(Kip1):FKBP-12 complex. In a specific embodiment, the present invention provides a method of using recombinant DNA techniques to express both p27(Kip1) and FKBP-12 (or a derivative, fragment or analog of one or both members of the complex) either where both binding partners are under the control of one heterologous promoter, i.e., a promoter not naturally associated with the gene encoding the particular complex component, or where each is under the control of a different heterologous promoter.

Methods of diagnosis, prognosis, and screening for diseases and disorders associated with aberrant levels of a p27(Kip1):FKBP-12 complex are also provided in the present invention. The present invention also provides methods of treating or preventing a disease or disorder associated with an aberrant level of a p27(Kip1):FKBP-12 complex, or an aberrant level of activity of one or more of the components of the complex, by administration of a p27(Kip1):FKBP-12 complex, or by administrating a modulator of p27(Kip1):FKBP-12 complex activity or formation, e.g., antibodies that bind to a p27(Kip1):FKBP-12 complex, or non-complexed p27(Kip1) or its binding partner or a fragment thereof. In this embodiment of the present invention, the fragment preferably contains the portion of p27(Kip1) or FKBP-12 that is directly involved in complex formation. In another embodiment, mutants of p27(Kip1) or of FKBP-12 that increase or decrease binding affinity, small molecule inhibitors or enhancers of complex formation, or antibodies that either stabilize or neutralize the complex, etc, can be administered.

Methods of assaying a p27(Kip1):FKBP-12 complex, for activity as a therapeutic or diagnostic, as well as methods of screening for a p27(Kip1):FKBP-12 complex modulator, i.e., agonists and antagonists, are also provided.

For clarity of disclosure, and not by way of limitation, the detailed description of the invention is divided into the following subsections.

5.1. p27(Kip1):FKBP-12 Complexes and Derivatives and Analogs Thereof

One embodiment of the present invention is directed to a p27(Kip1):FKBP-12 complex. In a preferred embodiment, the p27(Kip1):FKBP-12 complex is a complex of human proteins. The present invention is also directed to a complex of a derivative of p27(Kip1), including a fragment or analog thereof, with FKBP-12; a complex of p27(Kip1) with a derivative of FKBP-12, including a fragment or analog thereof; and a complex of a derivative of p27(Kip1) and a derivative of FKBP-12. As used herein, a fragment, derivative, homolog or analog of a p27(Kip1):FKBP-12 complex includes complexes where one or both members of the complex are fragments, derivatives or analogs of the wild-type p27(Kip1) or FKBP-12 protein. Preferably, the p27(Kip1):FKBP-12 complex in which one or both members of the complex is a fragment, derivative, homolog or analog of the wild type protein is a functionally active p27(Kip1):FKBP-12 complex. In particular aspects of this embodiment, the native proteins p27(Kip1) and/or FKBP-12, or a derivative or analog thereof, are obtained from animals, e.g., mouse, rat, pig, cow, dog, monkey, human, fly, frog. In another aspect, the native proteins or derivatives or analogs thereof are obtained from plants.

As used herein, a “functionally active p27(Kip1):FKBP-12 complex” refers to a complex displaying one or more functional attributes of a complex of wild type p27(Kip1) with wild type FKBP-12, including protein-protein binding, binding to a p27(Kip1)-, a FKBP-12-, and/or a p27(Kip1):FKBP-12 complex-specific antibody, or has the functional attribute(s) of p27(Kip1), FKBP-12, and/or a p27(Kip1):FKBP-12 complex involved in control of cell cycle progression, cellular differentiation and apoptosis, intracellular signal transduction, neurogenesis, response to viral infection, a hyperproliferative disorder such as tumorigenesis and tumor spread, a degenerative disorder such as a neurodegenerative disease, autoimmune disease, a disorder associated with organ transplantation, inflammatory and/or allergic disease, atherosclerosis, nephropathy, cardiac disease or muscle disease.

The present invention is also directed to a method of screening a p27(Kip1):FKBP-12 complex for the ability to alter a cell function, particularly those cell functions in which p27(Kip1) and/or FKBP-12 has been implicated, including physiological processes such as control of cell cycle progression, cellular differentiation and apoptosis, intracellular signal transduction, neurogenesis, response to viral infection; and pathophysiological processes including hyperproliferative disorders such as tumorigenesis and tumor spread, degenerative disorders such as neurodegenerative diseases, autoimmune diseases, disorders associated with organ transplantation, inflammatory and allergic diseases, atherosclerosis, nephropathy and cardiac and muscle diseases. The present invention is also directed to a method for screening a complex of a derivative or analog of p27(Kip1) and/or FKBP-12 for the ability to alter a cell function.

A specific embodiment of the present invention is directed to a p27(Kip1):FKBP-12 complex of a fragment of p27(Kip1) and/or a fragment of FKBP-12 that can be bound by an anti-p27(Kip1) antibody and/or bound by an anti-FKBP-12 antibody, respectively, or bound by an antibody specific for a p27(Kip1):FKBP-12 complex. A p27(Kip1):FKBP-12 complex containing a fragment of p27(Kip1) and/or of FKBP-12 or other derivatives or analogs of p27(Kip1) and/or FKBP-12 can be tested for a desired activity by procedures known in the art, including but not limited to the assays described in Sections 5.5 and 5.6, supra.

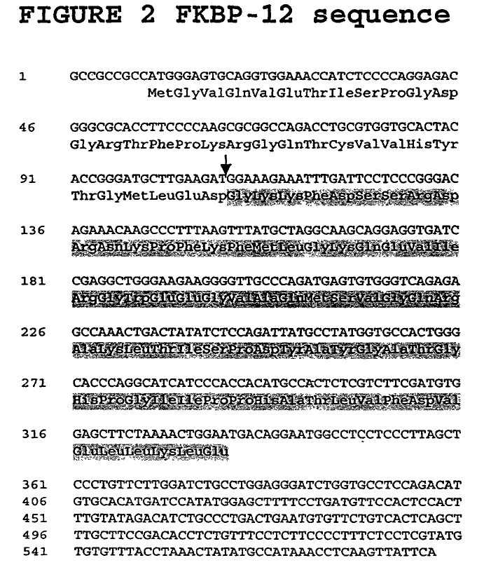

In specific embodiments, the present invention is directed to a p27(Kip1):FKBP-12 complex comprising a fragment of one or both members of the complex. In a preferred embodiment, these fragments consist of, but are not exclusive to fragments of FKBP-12, identified as interacting with p27(Kip1) in the modified yeast matrix mating assay, i.e., amino acids 34-107 of FKBP-12 as depicted in FIG. 2 (SEQ ID NO:4). Fragments, or proteins comprising fragments, lacking a region of either member of the complex, are also provided.

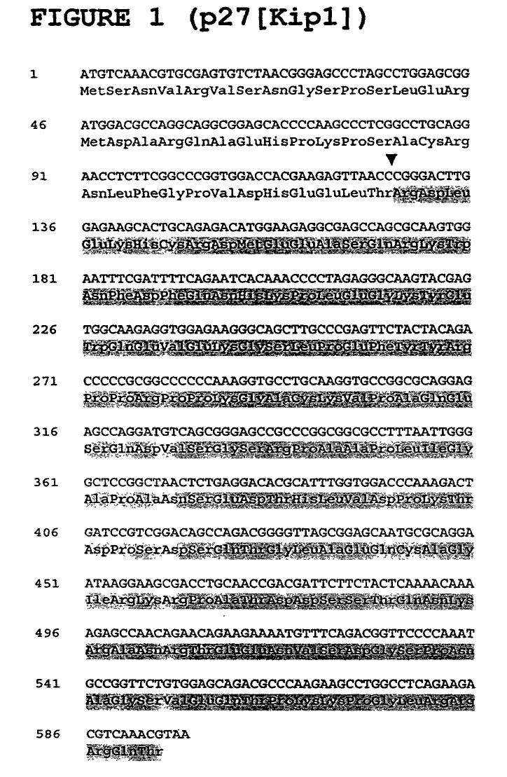

The nucleotide sequences encoding human p27(Kip1) and human FKBP-12 are known, (GenBank Accession No. U10906; GenBank Accession No. M80199, respectively), and are disclosed in FIGS. 1 and 2, SEQ ID NOS:1 and 3, respectively. Nucleic acid molecules encoding p27(Kip1) or FKBP-12 can be obtained by any method known in the art, e.g., by PCR amplification using synthetic primers hybridizable to the 3′ and 5′ ends of each sequence and/or by cloning from a cDNA or genomic library using an oligonucleotide specific for each nucleotide sequence.

Homologs (e.g., nucleic acid molecules encoding p27(Kip1) or FKBP-12 from a species other than human) or other related sequences, e.g., paralogs, can be obtained by low, moderate or high stringency hybridization with all or a portion of the particular human sequence as a probe using methods well known in the art for nucleic acid hybridization and cloning.

The p27(Kip1) and FKBP-12 proteins (the amino acid sequences are shown in FIGS. 1 and 2, SEQ ID NOS:2 and 4, respectively) either alone or in a complex, can be obtained by methods known in the art for protein purification and recombinant protein expression. For recombinant expression of one or more of the proteins, a nucleic acid molecule containing all or a portion of the nucleotide sequence encoding the protein can be inserted into an appropriate expression vector, i.e., a vector that contains the necessary elements for transcription and translation of the inserted protein coding sequence. The necessary transcriptional and translational signals may be supplied by the native promoter of the p27(Kip1) or FKBP-12 gene, and/or flanking regions.

A variety of host-vector systems may be utilized to express the protein coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

In a preferred embodiment, a p27(Kip1):FKBP-12 complex is obtained by expressing the entire p27(Kip1) coding sequence and the entire FKBP-12 coding sequence in the same cell, either under the control of the same promoter or two separate promoters. In yet another embodiment, a derivative, fragment or homolog of p27(Kip1) and/or a derivative, fragment or homolog of FKBP-12 are recombinantly expressed in the same cell. Preferably the derivative, fragment or homolog of p27(Kip1) and/or of FKBP-12 forms a complex with a binding partner identified by a binding assay, such as the modified yeast matrix mating test described in Section 5.6.1 infra, and more preferably forms a complex that binds to an anti-p27(Kip1):FKBP-12 complex antibody.

Any method available in the art can be used for the insertion of DNA fragments into a vector to construct expression vectors containing a chimeric gene consisting of appropriate transcriptional/translational control signals and protein coding sequences. These methods may include in vitro recombinant DNA and synthetic techniques and in vivo recombinant techniques (genetic recombination). Expression of nucleic acid sequences encoding p27(Kip1) or FKBP-12, or derivatives, fragments or homologs thereof, may be regulated by a second nucleic acid sequence so that the genes or fragments thereof are expressed in a host transformed with the recombinant DNA molecule(s). For example, expression of the proteins may be controlled by any promoter/enhancer known in the art. In a specific embodiment, the promoter is not native to the genes for p27(Kip1) or for FKBP-12. Promoters that may be used include but are not limited to the SV40 early promoter (Bernoist and Chambon, 1981, Nature 290: 304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42); prokaryotic expression vectors such as the β-lactamase promoter (Villa-Kamaroff et al., 1978, Proc. Natl. Acad. Sci. USA 75:3727-3731) or the tac promoter (DeBoer et al., 1983, Proc. Natl. Acad. Sci. USA 80:21-25; Gilbert et al., 1980, Scientific American 242:79-94); plant expression vectors comprising the nopaline synthetase promoter (Herrar-Estrella et al., 1984, Nature 303:209-213) or the cauliflower mosaic virus 35S RNA promoter (Garder et al., 1981, Nucleic Acids Res. 9:2871), and the promoter of the photosynthetic enzyme ribulose bisphosphate carboxylase (Herrera-Estrella et al., 1984, Nature 310:115-120); promoter elements from yeast and other fungi such as the Gal4 promoter (Johnston et al., 1987, Microbiol. Rev. 51:458-476), the alcohol dehydrogenase promoter (Schibler et al., 1987, Annual Review Genetics 21:237-257), the phosphoglycerol kinase promoter (Struhl et al., 1995, Annual Review Genetics 29:651-674-257; Guarente 1987, Annual Review Genetics 21:425-452), the alkaline phosphatase promoter (Struhl et al., 1995, Annual Review Genetics 29:651-674-257; Guarente 1987, Annual Review Genetics 21:425-452), and the following animal transcriptional control regions that exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald 1987, Hepatology 7:425-515); insulin gene control region which is active in pancreatic beta cells (Hanahan et al., 1985, Nature 315:115-122), immunoglobulin gene control region which is active in lymphoid cells (Grosschedl et al., 1984, Cell 38:647-658; Adams et al., 1985, Nature 318:533-538; Alexander et al., 1987,-Mol. Cell Biol. 7:1436-1444), mouse mammary tumor virus control region which is active in testicular, breast, lymphoid and mast cells (Leder et al., 1986, Cell 45:485-495), albumin gene control region which is active in liver (Pinckert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha-1 antitrypsin gene control region which is active in liver (Kelsey et al., 1987, Genes and Devel. 1:161-171), beta globin gene control region which is active in myeloid cells (Mogram et al., 1985, Nature 315:338-340; Kollias et al., 1986, Cell 46:89-94), myelin basic protein gene control region which is active in oligodendrocyte cells of the brain (Readhead et al., 1987, Cell 48:703-712), myosin light chain-2 gene control region which is active in skeletal muscle (Sani 1985, Nature 314:283-286), and gonadotrophic releasing hormone gene control region which is active in gonadotrophs of the hypothalamus (Mason et al., 1986, Science 234:1372-1378).

In a specific embodiment, a vector is used that comprises a promoter operably linked to a nucleic acid sequence encoding p27(Kip1) and/or FKBP-12, or a fragment, derivative or homolog thereof, one or more origins of replication, and optionally, one or more selectable markers (e.g., an antibiotic resistance gene). In a preferred embodiment, a vector is used that comprises a promoter operably linked to a nucleic acid sequence encoding both p27(Kip1) and FKBP-12, one or more origins of replication, and optionally, one or more selectable markers.

In another specific embodiment, an expression vector containing the coding sequence, or a portion thereof, of p27(Kip1) and FKBP-12, either together or separately, is made by subcloning the gene sequences into the EcoRI restriction site of each of the three pGEX vectors (glutathione S-transferase expression vectors; Smith and Johnson, 1988, Gene 7:31-40). This allows for the expression of products in the correct reading frame.

Expression vectors containing the sequences of interest can be identified by three general approaches: (a) nucleic acid hybridization, (b) presence or absence of “marker” gene function, and (c) expression of the inserted sequences. In the first approach, p27(Kip1) or FKBP-12 encoding nucleic acid sequences can be detected by nucleic acid hybridization to probes comprising sequences homologous and complementary to the encoding sequences. In the second approach, the recombinant vector/host system can be identified and selected based upon the presence or absence of certain “marker” functions (e.g., resistance to antibiotics, occlusion body formation in baculovirus, etc.) caused by insertion of the sequences of interest in the vector. For example, if a p27(Kip1) or FKBP-12 gene, or portion thereof, is inserted within the marker gene sequence of the vector, recombinants containing the p27(Kip1) or FKBP-12 fragment will be identified by the absence of the marker gene function (e.g., loss of beta-galactosidase activity). In the third approach, recombinant expression vectors can be identified by assaying for p27(Kip1) or FKBP-12 expressed by the recombinant vector. Such assays can be based, for example, on the physical or functional properties of the interacting species in in vitro assay systems, e.g., formation of a p27(Kip1):FKBP-12 complex or binding to an anti-p27(Kip1), anti-FKBP-12, or anti-p27(Kip1):FKBP-12 complex antibody.

Once recombinant p27(Kip1) and FKBP-12 molecules are identified and the complexes or individual proteins isolated, several methods known in the art can be used to propagate them. Once a suitable host system and growth conditions have been established, recombinant expression vectors can be propagated and amplified in quantity. As previously described, the expression vectors or derivatives which can be used include, but are not limited to: human or animal viruses such as vaccinia virus or adenovirus; insect viruses such as baculovirus, yeast vectors; bacteriophage vectors such as lambda phage; and plasmid and cosmid vectors.

In addition, a host cell strain may be chosen that modulates the expression of the inserted sequences, or modifies or processes the expressed proteins in the specific fashion desired. Expression from certain promoters can be elevated in the presence of certain inducers; thus expression of the genetically-engineered p27(Kip1) and/or FKBP-12 may be controlled. Furthermore, different host cells have characteristic and specific mechanisms for the translational and post-translational processing and modification (e.g., glycosylation, phosphorylation, etc.) of the expressed proteins. Appropriate cell lines or host systems can be chosen to ensure that the desired modification and processing of the expressed protein is achieved. For example, expression in a bacterial system can be used to produce an unglycosylated core protein, while expression in mammalian cells ensures “native” glycosylation of an expressed mammalian protein. Furthermore, different vector/host expression systems may effect processing reactions to a different extent.

In other specific embodiments, p27(Kip1) and/or FKBP-12 or fragments, homologs or derivatives thereof, may be expressed as a fusion or chimeric protein product comprising the protein, fragment, homolog, or derivative joined via a peptide bond to a heterologous protein sequence of a different protein. Such chimeric products can be made by ligating the appropriate nucleic acid sequence encoding the desired amino acid sequence to each other by methods known in the art, in the proper coding frame, and expressing the chimeric products in a suitable host by methods commonly known in the art. Alternatively, such a chimeric product can be made by protein synthetic techniques, e.g., by use of a peptide synthesizer. Chimeric genes comprising portions of p27(Kip1) and/or FKBP-12 fused to any heterologous protein-encoding sequence may be constructed. A specific embodiment of the present invention is directed to a chimeric protein comprising a fragment of p27(Kip1) and/or FKBP-12 of at least six amino acids.

In a specific embodiment, fusion proteins-are provided that contain the interacting domains of the p27(Kip1) protein and the FKBP-12 protein and, optionally, a peptide linker between the two domains, where such a linker promotes or at least does not disrupt or inhibit the interaction between the p27(Kip1) and FKBP-12 binding domains. These fusion proteins may be particularly useful here the stability of the interaction is desirable (due to he formation of the complex as an intra-molecular reaction), for example in production of antibodies specific for a 27(Kip1):FKBP-12 complex.

In particular, p27(Kip1) and/or FKBP-12 derivatives an be made by altering their sequences by substitutions, dditions or deletions that provide for functionally equivalent molecules. Due to the degeneracy of nucleotide coding sequences, other DNA sequences that encode substantially the same amino acid sequence as the native 27(Kip1) or FKBP-12 gene or cDNA can be used in the practice of the present invention. These include but are not limited o nucleotide sequences comprising all or portions of 27(Kip1) and FKBP-12 genes that are altered by the substitution of different codons that encode a functionally equivalent amino acid residue within the sequence, thus producing a silent change. Likewise, the p27(Kip1) and FKBP-12 derivatives of the invention include, but are not limited to, those containing, as a primary amino acid sequence, all or part of the amino acid sequence of p27(Kip1) or FKBP-12, including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment of the present invention, nucleic acid sequences encoding a protein or a protein consisting of or comprising a fragment of p27(Kip1) or FKBP-12 consisting of at least 6 (continuous) amino acids of p27(Kip1) or FKBP-12 are provided. In other embodiments, the fragment consists of at least 10, 20, 30, 40, or 50 amino acids of p27(Kip1) or FKBP-12. In specific embodiments, such fragments are not larger than 35, 100 or 200 amino acids. Derivatives or analogs of p27(Kip1) or FKBP-12 include, but are not limited to, molecules comprising regions that are substantially homologous to p27(Kip1) or to FKBP-12, in various embodiments, by at least 30%, 40%, 50%, 60%, 70%, 80%, 90% or 95% identity over an amino acid sequence of identical size or when compared to an aligned sequence in which the alignment is done by a computer homology program known in the art or whose encoding nucleic acid is capable of hybridizing to a sequence encoding p27(Kip1) or FKBP-12 under stringent, moderately stringent, or nonstringent conditions.

p27(Kip1) and FKBP-12 derivatives and analogs can be produced by various methods known in the art. The manipulations which result in their production can occur at the gene or protein level. For example, the cloned p27(Kip1) and FKBP-12 gene sequences can be modified by any of numerous strategies known in the art (Sambrook et al., 1989, Molecular Cloning, A Laboratory Manual, 2d Ed., Cold Spring Harbor Laboratory Press, Cold Spring Harbor, New York). The sequences can be cleaved at appropriate sites with restriction endonuclease(s), followed by further enzymatic modification if desired, isolated, and ligated in vitro. In the production of the gene encoding a derivative, homolog or analog of p27(Kip1) or FKBP-12, care should be taken to ensure that the modified gene retains the original translational reading frame, uninterrupted by translational stop signals, in the gene region where the desired activity is encoded.

Additionally, the p27(Kip1) and/or FKBP-12 encoding nucleic acid sequence can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Any technique for mutagenesis known in the art can be used, including but not limited to, chemical mutagenesis and in vitro site-directed mutagenesis (Hutchinson et al., 1978, J. Biol. Chem 253:6551-6558), use of TAB™ linkers (Pharmacia, Uppsala, Sweden), etc.

Once a recombinant cell expressing p27(Kip1) and/or FKBP-12, or fragment or derivative thereof, is identified, the individual gene product or complex can be isolated and analyzed. This is achieved by assays based on the physical and/or functional properties of the protein or complex, including, but not limited to, radioactive labeling of the product followed by analysis by gel electrophoresis, immunoassay, cross-linking to marker-labeled product, etc.

The p27(Kip1):FKBP-12 complex may be isolated and purified by standard methods known in the art (either from natural sources or recombinant host cells expressing the complex or member proteins), including but not restricted to column chromatography (e.g., ion exchange, affinity, gel exclusion, reversed-phase high pressure, fast protein liquid, etc.), differential centrifugation, differential solubility, or by any other standard technique used for the purification of proteins. Functional properties may be evaluated using any suitable assay known in the art.

Alternatively, once p27(Kip1) or its derivative, or FKBP-12 or its derivative is identified, the amino acid sequence of the protein can be deduced from the nucleic acid sequence of the chimeric gene from which it was encoded. As a result, the protein or its derivative can be synthesized by standard chemical methods known in the art (see, e.g., Hunkapiller et al., 1984, Nature 310:105-111).

In a specific embodiment of the present invention, such p27(Kip1):FKBP-12 complexes, whether produced by recombinant DNA techniques, chemical synthesis methods, or by purification from native sources, include but are not limited to those containing, as a primary amino acid sequence, all or part of the amino acid sequences substantially as depicted in FIGS. 1 and 2 (SEQ ID NOS:2 and 4, respectively), as well as fragments and other analogs and derivatives thereof, including proteins homologous thereto.

Manipulations of p27(Kip1) and/or FKBP-12 sequences ay be made at the protein level. Included within the scope of the invention are complexes of p27(Kip1) or FKBP-12 fragments, and p27(Kip1) or FKBP-12 fragments, derivatives or analogs that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formulation, oxidation, reduction, metabolic synthesis in the presence of tunicamycin, etc.

In specific embodiments, the p27(Kip1) and/or FKBP-12 amino acid sequences are modified to include a fluorescent label. In another specific embodiment, p27(Kip1) and/or FKBP-12 are modified to contain a heterofunctional reagent; such heterofunctional reagents can be used to crosslink the members of the complex.

In addition, complexes of analogs and derivatives of p27(Kip1) and/or FKBP-12 can be chemically synthesized. For example, a peptide corresponding to a portion of p27(Kip1) and/or FKBP-12, which comprises the desired domain or mediates the desired activity in vitro (e.g., p27(Kip1):FKBP-12 complex formation) can be synthesized by use of a peptide synthesizer. Furthermore, if desired, non-classical amino acids or chemical amino acid analogs can be introduced as a substitution or addition into the p27(Kip1) and/or FKBP-12 sequence. Non-classical amino acids include but are not limited to the D-isomers of the common amino acids, a-amino isobutyric acid, 4-aminobutyric acid (4-Abu), 2-aminobutyric acid (2-Abu), 6-amino hexanoic acid (Ahx), 2-amino isobutyric acid (2-Aib), 3-amino propionoic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Na-methyl amino acids, and amino acid 10 analogs in general. Furthermore, the amino acid can be D (dextrorotary) or L (levorotary).

In cases where natural products are suspected of being mutant or are isolated from new species, the amino acid sequence of p27(Kip1) or FKBP-12 isolated from the natural source, as well as those expressed in vitro, or from synthesized expression vectors in vivo or in vitro, can be determined from analysis of the DNA sequence, or alternatively, by direct sequencing of the isolated protein. Such analysis can be performed by manual sequencing or through use of an automated amino acid sequenator.

The p27(Kip1):FKBP-12 complexes of the present invention can also be analyzed by hydrophilicity analysis (Hopp and Woods, 1981, Proc. Natl. Acad. Sci. USA 78:3824-3828). A hydrophilicity profile can be used to identify the hydrophobic and hydrophilic regions of the proteins, and help predict their orientation in designing substrates for experimental manipulation, such as in binding experiments, antibody synthesis, etc. Secondary structural analysis can also be done to identify regions of p27(Kip1) and/or FKBP-12, or their derivatives, that form specific structures (Chou and Fasman, 1974, Biochemistry 13: 222-23). Manipulation, translation, secondary structure prediction, hydrophilicity and hydrophobicity profiles, open reading frame prediction and plotting, and determination of sequence homologies, can be accomplished using computer software programs available in the art. other methods of structural analysis including but not limited to X-ray crystallography (Engstrom, 1974 Biochem. Exp. Biol. 11:7-13), mass spectroscopy and gas chromatography (Methods in Protein Science, J. Wiley and Sons, New York, 1997), and computer modeling (Fletterick and Zoller, eds., 1986, Computer Graphics and Molecular Modeling, In: Current Communications in Molecular Biology, Cold Spring Harbor Laboratory, Cold Spring Harbor Press, New York) can also be employed.

5.2. Antibodies to p27(Kip1):FKBP-12 Complexes

According to the present invention, a p27(Kip1):FKBP-12 complex or a fragment, derivative and/or homolog thereof may be used as an immunogen to generate antibodies which immunospecifically bind the complex, fragment, derivative or homolog. Such antibodies include but are not limited to polyclonal, monoclonal, chimeric, single chain, Fab fragments, and an antibody obtained from a Fab expression library and can be purified antibodies. In a specific embodiment, antibodies to a complex of human p27(Kip1) and human FKBP-12 are produced. In another embodiment, a complex formed from a fragment of p27(Kip1) and FKBP-12, which fragments contain the protein domain that interacts with the-other member of the complex, are used as immunogens for antibody production. In yet another embodiment, the antibodies produced are specific for a complex of human proteins and not for a complex of proteins from another species.

Various procedures known in the art may be used for the production of polyclonal antibodies to a p27(Kip1):FKBP-12 complex, or to a fragment, derivative, homolog or analog thereof. For production of an antibody, various host animals can be immunized by injection with the native p27(Kip1):FKBP-12 complex or a synthetic version, or a derivative of the foregoing, such as a cross-linked p27(Kip1):FKBP-12 complex. Such host animals include but are not limited to rabbits, mice, rats, etc. Various adjuvants can be used to increase the immunological response, depending on the host species, and include but are not limited to Freund's adjuvant (complete and incomplete), mineral gels such as aluminum hydroxide, surface active substances such as lysolecithin, pluronic polyols, polyanions, peptides, oil emulsions, dinitrophenol, and potentially useful human adjuvants such as bacille Calmette-Guerin (BCG) and Corynebacterium parvum.

For preparation of a monoclonal antibody directed towards a p27(Kip1):FKBP-12 complex, or a fragment, derivative, homolog or analog thereof, any technique that provides for the production of antibody molecules by continuous cell lines in culture may be used. Such techniques include but are not restricted to the hybridoma technique originally developed by Kohler and Milstein, 1975, Nature 256:495-497, the trioma technique (Gustafsson et al., 1991, Hum. Anitbodies. Hybridomas 2:26-32), the human B-cell hybridoma technique (Kozbor et al., 1983, Immunology Today 4:72), and the EBV hybridoma technique to produce human monoclonal antibodies (Cole et al., 1985, In: Monoclonal Antibodies and Cancer Therapy, Alan R. Liss, Inc., pp. 77-96). In another embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. USA 80:2026-2030) or by transforming human B cells with EBV virus in vitro (Cole et al., 1985, In: Monoclonal Antibodies and Cancer Therapy, Alan R. Liss, Inc., pp. 77-96). In fact, according to the invention, techniques developed for the production of “chimeric antibodies” by splicing the genes from a mouse antibody molecule specific for the p27(Kip1):FKBP-12 complex together with genes from a human antibody molecule of appropriate biological activity can be used; such antibodies are within the scope of this invention. Morrison et al., 1984, Proc. Natl. Acad. Sci. USA 81:6851-6855; Neuberger et al., 1984, Nature 312:604-608; and Takeda et al., 1985, Nature 314:452-454.

According to the invention, techniques described for the production of single chain antibodies (U.S. Pat. No. 4,946,778) can be adapted to produce p27(Kip1):FKBP-12 complex-specific antibodies. Another embodiment of the invention utilizes the techniques described for the construction of Fab expression libraries (Huse et al., 1989, Science 246:1275-1281) to allow rapid and easy identification of monoclonal Fab′ fragments with the desired specificity for p27(Kip1):FKBP-12 complexes, derivatives, or analogs. Non-human antibodies can be “humanized” by known methods, see, e.g., U.S. Pat. No. 5,225,539.

Antibody fragments that contain the idiotypes of p27(Kip1):FKBP-12 complexes can be generated by techniques known in the art. For example, such fragments include but are not limited to: the F(ab′)2 fragment which can be produced by pepsin digestion of the antibody molecule; the Fab′ fragments that can be generated by reducing the disulfide bridges of the F(ab′)2 fragment; the Fab fragments that can be generated by treating the antibody molecular with papain and a reducing agent; and Fv fragments.

In the production of antibodies, screening for the desired antibody can be accomplished by techniques known in the art, e.g., ELISA (enzyme-linked immunosorbent assay). To select antibodies specific to a particular domain of the p27(Kip1):FKBP-12 complex, or a derivative, homolog, or analog thereof, one may assay generated hybridomas for a product that binds to a fragment of the p27(Kip1):FKBP-12 complex, or a derivative, homolog, or analog thereof, that contains such a domain. For selection of an antibody that specifically binds a p27(Kip1):FKBP-12 complex, or a derivative, homolog, or analog thereof, but which does not specifically bind to the individual proteins of the p27(Kip1):FKBP-12 complex, or a derivative, homolog, or analog thereof, one can select on the basis of positive binding to the p27(Kip1):FKBP-12 complex and a lack of binding to the individual p27(Kip1) and FKBP-12 proteins.

Antibodies specific to a domain of a p27(Kip1):FKBP-12 complex, or a derivative, homolog, or analog thereof, are also provided.

The foregoing antibodies can be used in methods known in the art relating to the localization and/or quantitation of a p27(Kip1):FKBP-12 complex, e.g., for imaging, measuring levels of the complex in an appropriate physiological sample, in diagnostic methods, etc. This holds true also for a derivative, homolog, or analog of a p27(Kip1):FKBP-12 complex.

In another embodiment of the invention (see infra), anti-p27(Kip1):FKBP-12 complex antibodies and fragments containing the binding domain thereof are used as therapeutics.

5.3. Diagnostic, Prognostic, and Screening Uses of a p27(KiP1):FKBP-12 Complex

p27(Kip1):FKBP-12 complexes are involved in normal physiological processes including, but not limited to, control of cell cycle progression, cellular differentiation and apoptosis, intracellular signal transduction, neurogenesis, response to viral-infection; and pathophysiological processes including, but not limited to, hyperproliferative disorders such as tumorigenesis and tumor spread, degenerative disorders such as neurodegenerative diseases, autoimmune diseases, disorders associated with organ transplantation, inflammatory and allergic diseases, atherosclerosis, nephropathy and cardiac and muscle diseases. Thus, the complexes have diagnostic utility by measuring the levels as compared to “normal” individuals, with aberrant levels being a marker of a disease or disorder. Further, definition of particular groups of patients with increased or decreased levels of a p27(Kip1):FKBP-12 complex can lead to new nosological classifications of diseases, furthering diagnostic ability. In addition, detecting levels of a p27(Kip1):FKBP-12 complex, or individual constituent proteins that have been shown to form complexes with p27(Kip1), or detecting levels of the mRNAs encoding the components of a p27(Kip1):FKBP-12 complex may be used in prognosis, to follow the course of a disease state, to follow therapeutic response, etc.

p27(Kip1):FKBP-12 complexes and the individual components of the p27(Kip1):FKBP-12 complexes, i.e., p27(Kip1) and FKBP-12, and derivatives, analogs and fragments thereof; p27(Kip1) and/or FKBP-12 encoding nucleic acid sequences, and sequences complementary thereto; and anti-p27(Kip1):FKBP-12 complex antibodies and antibodies directed against the individual components that can form a p27(Kip1):FKBP-12 complex, have uses in diagnostic assays. The foregoing molecules can be used in assays, such as immunoassays, to detect, prognoses diagnose, or monitor various conditions, diseases, or disorders characterized by aberrant levels of a p27(Kip1):FKBP-12 complex, or monitor the treatment of such diseases.

In particular, such an immunoassay is carried out by a method comprising contacting a sample obtained from a patient with an anti-p27(Kip1):FKBP-12 complex antibody under conditions such that immunospecific binding can occur, and detecting or measuring the amount of any immunospecific binding by the antibody. In a specific aspect, such binding of antibody, in tissue sections, can be used to detect aberrant p27(Kip1):FKBP-12 complex localization, or aberrant, i.e., high, low or absent, levels of a p27(Kip1):FKBP-12 complex or complexes. In a specific embodiment, an antibody to a p27(Kip1):FKBP-12 complex can be used to assay for the presence of a p27(Kip1):FKBP-12 complex in a patient tissue or serum sample, where an aberrant level of the p27(Kip1):FKBP-12 complex is an indication of a diseased condition. An “aberrant level” means an increased or decreased level relative to that present, or a standard level representing that present, in an analogous sample from a portion or fluid of the body, or from a subject not having the disorder.

The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few known in the art.

Nucleic acid sequences encoding the components of a p27(Kip1):FKBP-12 complex, i.e., p27(Kip1) and FKBP-12, and related nucleic acid sequences and subsequences, including complementary sequences, can be used in hybridization assays. The p27(Kip1) and FKBP-12 encoding nucleic acid sequences, or subsequences thereof comprising about at least 8 nucleotides, can be used as hybridization probes. Hybridization assays can be used to detect, prognose, diagnose, or monitor conditions, disorders, or disease states associated with aberrant levels of the mRNAs encoding the components of a p27(Kip1):FKBP-12 complex as described, supra. In particular, such a hybridization assay comprises contacting a sample containing a nucleic acid sequence with a nucleic acid probe capable of hybridizing to p27(Kip1) or to FKBP-12 DNA or RNA, under conditions such that hybridization can occur, and detecting or measuring any resulting hybridization. In a preferred aspect, the hybridization assay is carried out using nucleic acid probes capable of hybridizing to p27(Kip1) and to a binding partner of p27(Kip1), e.g., FKBP-12, to measure concurrently the expression of members of a p27(Kip1):FKBP-12 complex.

In specific embodiments of the present invention, diseases and disorders involving or characterized by aberrant levels of a p27(Kip1):FKBP-12 complex can be diagnosed, or their suspected presence can be screened for, or a predisposition to develop such disorders can be detected by detecting aberrant levels of a p27(Kip1):FKBP-12 complex, or by detecting aberrant levels of un-complexed p27(Kip1) and/or FKBP-12 proteins or their encoding nucleic acid sequences, or by detecting functional activity including, but not limited to, binding to an interacting partner, e.g., p27(Kip1) or FKBP-12, or by detecting mutations in p27(Kip1) and/or FKBP-12 or their encoding RNA or DNA, e.g., translocations, truncations, changes in nucleotide or amino acid sequence relative to wild-type p27(Kip1) and/or FKBP-12, that cause increased or decreased expression or activity of a p27(Kip1):FKBP-12 complex, or p27(Kip1), or a protein that binds to p27(Kip1), e.g., FKBP-12. Such diseases and disorders include but are not limited to those described in Section 5.4 and its subsections infra.

By way of example, levels of a p27(Kip1):FKBP-12 complex and the individual components of a p27(Kip1):FKBP-12 complex can be detected by immunoassay, levels of p27(Kip1) and/or FKBP-12 RNA can be detected by hybridization assays, e.g., Northern blots, dot blots, and binding of p27(Kip1) to FKBP-12 can be measured by binding assays commonly known in the art. Translocations and point mutations in p27(Kip1) and/or FKBP-12 genes can be detected by Southern blotting, RFLP analysis, PCR using primers that preferably generate a fragment spanning at least most of the p27(Kip1) and/or FKBP-12 gene, by sequencing p27(Kip1) and/or FKBP-12 genomic DNA or cDNA obtained from a patient, etc.

Assays well known in the art, e.g., assays described above such as immunoassays, nucleic acid hybridization assays, activity assays, etc., can be used to determine whether one or more particular p27(Kip1):FKBP-12 complexes are present at either increased or decreased levels, or are absent, in samples from patients suffering from a particular disease or disorder, or having a predisposition to develop such a disease or disorder, as compared to the levels in samples from subjects not having such a disease or disorder, or not having a predisposition to develop such a disease or disorder. Additionally, these assays can be used to determine whether the ratio of the p27(Kip1):FKBP-12 complex to the un-complexed components of the p27(Kip1):FKBP-12 complex, i.e, p27(Kip1) and/or FKBP-12, is increased or decreased in samples from patients suffering from a particular disease or disorder, or having a predisposition to develop such a disease or disorder, as compared to the ratio in samples from subjects not having such a disease or disorder or not having such predisposition. In the event that levels of a p27(Kip1):FKBP-12 complex, or one or more particular p27(Kip1):FKBP-12 complexes, i.e., complexes formed from p27(Kip1) and/or FKBP-12 derivatives, homologs, fragments, or analogs, are determined to be increased in patients suffering from a particular disease or disorder or having a predisposition to develop such a disease or disorder, then the particular disease or disorder or predisposition for a disease or disorder can be diagnosed, have prognosis defined for, be screened for, or be monitored by detecting increased levels of the one or more p27(Kip1):FKBP-12 complexes, or by detecting increased levels of mRNA encoding for a member of one or more particular p27(Kip1):FKBP-12 complexes, or by detecting increased p27(Kip1):FKBP-12 complex functional activity.

Accordingly, in a specific embodiment of the present invention, diseases and disorders involving increased levels of one or more p27(Kip1):FKBP-12 complexes can be diagnosed, or their suspected presence can be screened for, or a predisposition to develop such disorders can be detected by detecting increased levels of the one or more p27(Kip1):FKBP-12 complexes, by detecting increased mRNA encoding any members of the complex, or by detecting increased complex functional activity, or by detecting mutations in p27(Kip1) or FKBP-12, e.g., translocations in nucleic acids, truncations in the gene or protein, changes in nucleotide or amino acid sequence relative to wild-type p27(Kip1) or FKBP-12, that increase p27(Kip1):FKBP-12 complex formation.

In the event that levels of one or more particular p27(Kip1):FKBP-12 complexes are determined to be decreased in patients suffering from a particular disease or disorder or having a predisposition to develop such a disease or disorder, then the particular disease or disorder or predisposition for a disease or disorder can be diagnosed, have its prognosis determined, be screened for, or be monitored by detecting decreased levels of the one or more p27(Kip1):FKBP-12 complexes, by detecting decreased mRNA encoding for a member of the particular one or more p27(Kip1):FKBP-12 complexes, or by detecting decreased p27(Kip1):FKBP-12 complex functional activity.

Accordingly, in a specific embodiment of the present invention, diseases and disorders involving decreased levels of one or more p27(Kip1):FKBP-12 complexes can be diagnosed, or their suspected presence can be screened for, or a predisposition to develop such disorders can be detected by detecting decreased levels of the one or more p27(Kip1):FKBP-12 complexes, by detecting decreased levels of mRNA encoding a member of the one or more particular complexes, or by detecting a decreased in complex functional activity, or by detecting mutations in p27(Kip1) or FKBP-12, e.g., translocations in nucleic acids, truncations in the gene or protein, changes in nucleotide or amino acid sequence relative to wild-type p27(Kip1) or the FKBP-12, that decrease p27(Kip1):FKBP-12 complex formation.

The use of detection techniques, especially those involving antibodies against a p27(Kip1):FKBP-12 complex, provides a method of detecting specific cells that express the complex or a constitute protein. Using such assays, specific cell types can be defined in which one or more particular p27(Kip1):FKBP-12 complexes are expressed, and the presence of the complex or protein can be correlated with cell viability.

Also embodied in the present invention are methods to detect a p27(Kip1):FKBP-12 complex in cell culture models that express a particular p27(Kip1):FKBP-12 complex or derivative thereof, for the purpose of characterizing or preparing a p27(Kip1):FKBP-12 complex for harvest. This embodiment includes cell sorting of prokaryotes such as but not restricted to bacteria (Davey and Kell, 1996, Microbiol. Rev. 60:641-696), primary cultures and tissue specimens from eukaryotes, including mammalian species such as human (Steele et al., 1996, Clin. Obstet. Gynecol 39:801-813), and continuous cell cultures (Orfao and Ruiz-Arguelles, 1996, Clin. Biochem. 29:5-9). Such isolations can also be used in methods of diagnosis, described supra.

Kits for diagnostic use are also provided in the present invention, that comprise in one or more containers an anti-p27(Kip1):FKBP-12 complex antibody, and, optionally, a labeled binding partner to the antibody. Alternatively, the anti-p27(Kip1):FKBP-12 complex antibody, can be labeled with a detectable marker, e.g., a chemiluminescent, enzymatic, fluorescent, or radioactive moiety. A kit is also provided in the present invention that comprises in one or more containers a nucleic acid probe capable of hybridizing to p27(Kip1) and/or FKBP-12 DNA or RNA, e.g., FKBP-12 encoding mRNA. In a specific embodiment, a kit can comprise in one or more containers a pair of primers, e.g., each in the size range of 6-30 nucleotides, that are capable of priming for amplification by, e.g., polymerase chain reaction (see, e.g., Innis et al., 1990, PCR Protocols, Academic Press, Inc., San Diego, Calif.), ligase chain reaction (see, e.g., EP 320,308), use of Qβ replicase, cyclic probe reaction, or other methods known in the art, under appropriate reaction conditions of at least a portion of a p27(Kip1) and/or FKBP-12 encoding nucleic acid molecule. A kit can optionally further comprise a predetermined amount of a purified p27(Kip1):FKBP-12 complex, p27(Kip1) and/or FKBP-12 protein, or a nucleic acid molecule encoding p27(Kip1) and/or FKBP-12 for use as a standard or control.

5.4. Therapeutic Uses of p27(Kip1):FKBP-12 Complexes

The present invention provides a method for treatment or prevention of various diseases and disorders by administration of a therapeutic compound (termed herein “Therapeutic”). Such “Therapeutics” include but are not limited to: a p27(Kip1):FKBP-12 complex, the individual p27(Kip1) or FKBP-12 protein and analogs and derivatives, including fragments of the foregoing; antibodies thereto; nucleic acid sequences encoding p27(Kip1) and/or FKBP-12, and analogs or derivatives, thereof; p27(Kip1) and FKBP-12 antisense nucleic acids, and agents that modulate p27(Kip1):FKBP-12 complex activity or formation, i.e., agonists and antagonists).

As discussed in Section 2 supra, p27(Kip1) and its binding partner FKBP-12, are implicated significantly in disorders of cell cycle progression and cell differentiation, including cancer and tumorigenesis and tumor progression. Disorders of neurodegeneration resulting from altered cellular apoptosis, differentiation, and DNA repair likewise involves these same proteins. A wide range of cell diseases affected by physiological processes such as control of cell cycle progression, cellular differentiation and apoptosis, intracellular signal transduction, neurogenesis, response to viral infection; and pathophysiological processes including but not limited to hyperproliferative disorders such as tumorigenesis and tumor spread, degenerative disorders such as neurodegenerative diseases, autoimmune diseases, disorders associated with organ transplantation, inflammatory and allergic diseases, atherosclerosis, nephropathy and cardiac and muscle diseases are treated or prevented by administration of a Therapeutic that modulates, i.e., antagonizes or promotes) p27(Kip1):FKBP-12 complex activity or formation.

Diseases or disorders associated with aberrant levels of a p27(Kip1):FKBP-12 complex or levels of activity or aberrant levels of p27(Kip1) may be treated by administration of a Therapeutic that modulates p27(Kip1):FKBP-12 complex formation or activity. In a specific embodiment, the activity or levels of p27(Kip1) are modulated by administration of FKBP-12. In another specific embodiment, the activity or levels of FKBP-12 are modulated by administration of p27(Kip1).

Diseases and disorders characterized by increased (relative to a subject not suffering from the disease or disorder) p27(Kip1):FKBP-12 complex levels or activity can be treated with Therapeutics that antagonize, i.e., reduce or inhibit, p27(Kip1):FKBP-12 complex formation or activity. Therapeutics that can be used include but are not limited to p27(Kip1) or FKBP-12 or analogs, derivatives or fragments thereof; anti-p27(Kip1):FKBP-12 complex antibodies, e.g., antibodies specific for p27(Kip1):FKBP-12, or fragments and derivatives containing the binding region thereof; nucleic acid molecules encoding p27(Kip1) or FKBP-12; p27(Kip1) and/or FKBP-12 antisense nucleic acid; and p27(Kip1) and/or FKBP-12 nucleic acid molecules that are dysfunctional, e.g., due to a heterologous (non-p27(Kip1) and/or non-FKBP-12) insertion within the coding sequences of the p27(Kip1) or FKBP-12 coding sequences that are used to “knockout” endogenous p27(Kip1) and/or FKBP-12 function by homologous recombination.

In a specific embodiment of the invention, a nucleic acid molecule, that contains a portion of the p27(Kip1) and/or FKBP-12 gene in which p27(Kip1) and FKBP-12 sequences flank a different gene sequence, is used as a p27(Kip1) and/or FKBP-12 antagonist, or is used to promote p27(Kip1) and/or FKBP-12 inactivation by homologous recombination. Koller and Smithies, 1989, Proc. Natl. Acad. Sci. USA 86:8932-8935; Zijlstra et al., 1989, Nature 342: 435-438. Additionally, mutants or derivatives of a wild type FKBP-12 protein that has greater affinity for p27(Kip1) than the wild type FKBP-12 may be administered to compete with wild type FKBP-12 protein.for p27(Kip1) binding, thereby reducing the levels of p27(Kip1) complexes with wild type FKBP-12. Other Therapeutics that inhibit p27(Kip1):FKBP-12 complex function can be identified by use of known convenient in vitro assays based on their ability to inhibit p27(Kip1):FKBP-12 complex formation, or as described in Section 5.5, infra.

In specific embodiments, Therapeutics that antagonize p27(Kip1):FKBP-12 complex formation or activity are administered therapeutically (including prophylactically): (1) in diseases or disorders involving an increased (relative to normal or desired) level of a p27(Kip1):FKBP-12 complex, for example, in patients where the p27(Kip1):FKBP-12 complex is overactive or overexpressed; or (2) in diseases or disorders wherein in vitro or in vivo assays indicate the utility of p27(Kip1):FKBP-12 antagonist administration. Increased levels of a p27(Kip1):FKBP-12 complex can be readily detected, e.g., by quantifying protein and/or RNA, by obtaining a patient tissue sample (e.g., from biopsy tissue) and assaying the sample in vitro for RNA or protein levels, structure and/or activity of the expressed p27(Kip1):FKBP-12 complex or of the p27(Kip1) and FKBP-12 mRNA. Many methods which are standard in the art can be employed, including but not limited to, immunoassays to detect a p27(Kip1):FKBP-12 complex and/or visualize a p27(Kip1):FKBP-12 complex (e.g., Western blot, immunoprecipitation followed by sodium dodecyl sulfate polyacrylamide gel electrophoresis, immunocytochemistry, etc.) and/or hybridization assays to detect concurrent expression of p27(Kip1) and FKBP-12 mRNA (e.g., Northern assays, dot blots, in situ hybridization, etc.).

A more specific embodiment includes methods of reducing p27(Kip1):FKBP-12 complex expression (i.e., the expression of the two components of the p27(Kip1):FKBP-12 complex and/or formation of the complex) by targeting mRNAs that express the protein moieties. RNA therapeutics currently fall within three classes, antisense species, ribozymes, or RNA aptamers. Good et al., 1997, Gene Therapy 4:45-54.

Antisense oligonucleotides have been the most widely used. By way of example, but not for limitation, antisense oligonucleotide methodology to reduce p27(Kip1):FKBP-12 complex formation is presented below in subsection 5.4.9, infra. Ribozyme therapy involves the administration, induced expression, etc., of small RNA molecules with enzymatic ability to cleave, bind, or otherwise inactivate specific RNAs to reduce or eliminate expression of particular proteins. Grassi and Marini, 1996, Annals of Medicine 28:499-510; Gibson, 1996, Cancer and Metastasis Reviews 15:287-299. At present, the design of “hairpin” and “hammerhead” RNA ribozymes is necessary to specifically target a particular mRNA such as that for p27(Kip1). RNA aptamers are specific RNA ligands for proteins, such as for Tat and Rev RNA (Good et al., 1997, Gene Therapy 4:45-54) that can specifically inhibit their translation. Aptamers specific for p27(Kip1) or FKBP-12 can be identified by many methods well known in the art, for example but not limited to the protein-protein interaction assay described in Section 5.6.1 infra.

In another embodiment, the activity or levels of p27(Kip1) are reduced by administration of FKBP-12, or a nucleic acid that encodes the FKBP-12, or antibody that immunospecifically binds to p27(Kip1), or a fragment or a derivative of the antibody containing the binding domain thereof. Additionally, the levels or activity of FKBP-12 may be reduced by administration of p27(Kip1) or a nucleic acid that encodes p27(Kip1), or an antibody that immunospecifically binds FKBP-12 or a fragment or derivative of the antibody containing the binding domain thereof.

In another aspect of the invention, diseases or disorders associated with increased levels of p27(Kip1) or increased levels of FKBP-12 may be treated or prevented by administration of a Therapeutic that increases p27(Kip1):FKBP-12 complex.formation if the complex formation acts to reduce or inactivate p27(Kip1) or FKBP-12 through p27(Kip1):FKBP-12 complex formation. Such diseases or disorders can be treated or prevented by administration of one member of the p27(Kip1):FKBP-12 complex, including mutants of a member of the p27(Kip1):FKBP-12 that has increased affinity for the other member of the p27(Kip1):FKBP-12 complex (to cause increased complex formation), administration of antibodies or other molecules that stabilize the p27(Kip1):FKBP-12 complex, etc.

Diseases and disorders associated with underexpression of a p27(Kip1):FKBP-12, or p27(Kip1) or FKBP-12 are treated or prevented by administration of a Therapeutic that promotes (i.e., increases or supplies) p27(Kip1):FKBP-12 complex activity or formation or individual p27(Kip1) or FKBP-12 activity. Examples of such a Therapeutic include but are not limited to p27(Kip1):FKBP-12 complexes and derivatives, analogs and fragments thereof that are functionally active (e.g., able to form a p27(Kip1):FKBP-12 complex), un-complexed p27(Kip1) or FKBP-12 proteins and derivatives, analogs, and fragments thereof, and nucleic acid sequences encoding the members of a p27(Kip1):FKBP-12 complex or functionally active derivatives or fragments thereof. In a specific embodiment of the present invention, Therapeutics are derivatives, homologs or fragments of p27(Kip1) and/or FKBP-12 that increase and/or stabilize p27(Kip1):FKBP-12 complex formation. Examples of other agonists can be identified using in vitro assays or animal models, examples of which are described in section 5.6 and 5.8, infra.

In specific embodiments, Therapeutics that promote p27(Kip1):FKBP-12 complex function are administered therapeutically (including prophylactically): (1) in diseases or disorders involving an absence or decreased (relative to normal or desired) level of a p27(Kip1):FKBP-12 complex, for example, in patients where p27(Kip1):FKBP-12 complexes (or the individual components necessary to form the complexes) are lacking, genetically defective, biologically inactive or underactive, or under-expressed; or (2) in diseases or disorders wherein in vitro (or in vivo) assays (see infra) indicate the utility of p27(Kip1):FKBP-12 complex agonist administration. The absence or decreased level of p27(Kip1):FKBP-12 complex, protein or function can be readily detected, e.g., by obtaining a patient tissue sample (e.g., from biopsy tissue) and assaying it in vitro for RNA or protein levels, structure and/or activity of the expressed p27(Kip1):FKBP-12 complex (or for the concurrent expression of mRNA encoding the two components of the p27(Kip1):FKBP-12 complex). Many methods standard in the art can be thus employed, including but not limited to immunoassays to detect and/or visualize a p27(Kip1):FKBP-12 complex (or the individual components of a p27(Kip1):FKBP-12 complex (e.g., Western blot, immunoprecipitation followed by sodium dodecyl sulfate polyacrylamide gel electrophoresis, immunocytochemistry, etc.) and/or hybridization assays to detect expression of the mRNA encoding the individual protein components of the p27(Kip1):FKBP-12 complex by detecting and/or visualizing p27(Kip1) and FKBP-12 mRNA concurrently or separately using, e.g., Northern assays, dot blots, in situ hybridization, etc.

In specific embodiments, the activity or levels of p27(Kip1) are increased by administration of FKBP-12, or a derivative, homolog or analog thereof, a nucleic acid encoding FKBP-12, or an agent that stabilizes or enhances FKBP-12, or a fragment or derivative of such an agent having such stabilizing or enhancing ability. In another specific embodiment, the activity or levels of FKBP-12 are increased by administration of p27(Kip1), or derivative, homolog or analog thereof, a nucleic acid encoding p27(Kip1), or an agent that stabilizes or enhances p27(Kip1), or a fragment or derivative of such an agent having such stabilizing or enhancing activity.

Generally, administration of products of species origin or species reactivity (in the case of antibodies) that is the same species as that of the patient is preferred. Thus, in a preferred embodiment, a human p27(Kip1):FKBP-12 complex, or derivative, homolog or analog thereof, nucleic acid molecules encoding the members of the human p27(Kip1):FKBP-12 complex or derivative, homolog or analog thereof, an antibody to a human p27(Kip1):FKBP-12 complex, or derivative thereof, or other human agents that affect p27(Kip1) and/or FKBP-12 expression or ability to form a p27(Kip1):FKBP-12 complex, are therapeutically or prophylactically administered to a human patient.

Preferably, suitable in vitro or in vivo assays are utilized to determine the effect of a specific Therapeutic and whether its administration is indicated for treatment of an affected tissue.

In various specific embodiments, in vitro assays can be carried out with representative cells of cell types involved in a patient's disorder, to determine if a Therapeutic has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used, as described in section 5.8. Additional descriptions and sources of Therapeutics that can be used according to the present invention are found in Sections 5.1-5.3 and 5.7 herein.

5.4.1. Malignancies

Components of the p27(Kip1):FKBP-12 complex have been implicated in regulation of cell proliferation. Accordingly, Therapeutics of the present invention may be useful in treating or preventing diseases or disorders associated with cell hyperproliferation or loss of control of cell proliferation, particularly cancers, malignancies and tumors. Therapeutics of the invention can be assayed by any method known in the art for efficacy in treating or preventing malignancies and related disorders. Such assays include in vitro assays using transformed cells or cells derived from the tumor of a patient or in vivo assays using animal models of cancer or malignancies, or any of the assays described in Sections 5.5, 5.6, and 5.8, infra. Potentially effective Therapeutics, for example, include agents that inhibit proliferation of tumors or transformed cells in culture, or cause regression of tumors in animal models in comparison to controls, e.g., as described in Section 5.5, 5.6 and 5.8, infra.

Accordingly, once a malignancy or cancer has been shown to be amenable to treatment by modulation of (i.e., to antagonize or agonize) p27(Kip1):FKBP-12 complex activity, that cancer or malignancy can be treated or prevented by administration of a Therapeutic that modulates p27(Kip1):FKBP-12 complex activity or formation, including supplying a p27(Kip1):FKBP-12 complex or the individual binding partners of a p27(Kip1):FKBP-12 complex, e.g., p27(Kip1) and FKBP-12, and derivatives or fragments of p27(Kip1) and FKBP-1 that modify p27(Kip1):FKBP-12 complex activity or formation. Such cancers and malignancies include but are not limited to those listed in Table 1 (for a review of such disorders, see Fishman et al., 1985, Medicine, 2d Ed., J. B. Lippincott Co., Philadelphia).

TABLE 1
MALIGNANCIES AND RELATED DISORDERS

Leukemia

acute leukemia

acute lymphocytic leukemia
acute myelocytic leukemia

myeloblastic
promyelocytic
myelomonocytic
monocytic
erythroleukemia

chronic leukemia

chronic myelocytic (granulocytic) leukemia
chronic lymphocytic leukemia

Polycythemia vera
Lymphoma

Hodgkin's disease
non-Hodgkin's disease

Multiple myeloma
Waldenstrom's macroglobulinemia
Heavy chain disease
Solid tumors

sarcomas and carcinomas

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular tumor
lung carcinoma
small cell lung carcinoma
bladder carcinoma
epithelial carcinoma
glioma
astrocytoma
medulloblastoma
craniopharyngioma
ependymoma
pinealoma
hemangioblastoma
acoustic neuroma
oligodendroglioma
meningioma
melanoma
neuroblastoma
retinoblastoma

In specific embodiments, malignancy or dysproliferative changes, such as metaplasias and dysplasias, or hyperproliferative disorders, are treated or prevented in the bladder, brain, breast, colon, lung, ovary, pancreas, prostate, testicle, stomach, or uterus.

5.4.2. Premalignant Conditions

Therapeutics of the invention that are effective in treating cancer or malignancies (e.g., as described above) can also be administered to treat premalignant conditions and to prevent progression to a neoplastic or malignant state, including but not limited to those disorders listed in Table 1. Such prophylactic or therapeutic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and-Angell, 1976, Basic Pathology, 2d Ed., W.B. Saunders Co., Philadelphia, pp. 68-79). Hyperplasia is a form of controlled cell proliferation involving an increase in cell number in a tissue or organ, without significant alteration in structure or function. As an example, endometrial hyperplasia often precedes endometrial cancer. Metaplasia is a form of controlled cell growth in which one type of adult cell or fully differentiated cell substitutes for another type of adult cell. Metaplasia can occur in epithelial or connective tissue cells, preceding malignant transformation, in, for example, squamous epithelium. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder, as a premalignant condition.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of a Therapeutic of the invention that modulates p27(Kip1):FKBP-12 complex activity. As mentioned supra, such characteristics of a transformed phenotype include morphological changes, loosening of substratum attachment, loss of contact inhibition, loss of anchorage dependence, protease release, increased sugar transport, decreased serum requirement for growth or survival, expression of fetal antigens, disappearance of the 250,000 dalton cell surface protein, etc. (see also Id., pp. 84-90 for characteristics associated with a transformed or malignant phenotype).

In a specific embodiment, leukoplakia, a benign-appearing hyperplastic or dysplastic lesion of the oral epithelium, or Bowen's disease, a carcinoma in situ, are pre-neoplastic lesions indicative of the desirability of prophylactic intervention.

In another specific embodiment, fibrocystic disease, cystic hyperplasia, and mammary dysplasia, particularly adenosis (benign epithelial hyperplasia) are indicative of the desirability of prophylactic intervention.

In other embodiments, a patient that exhibits one or more of the following predisposing factors for malignancy is treated by administration of an effective amount of a Therapeutic: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon tumorigenesis), benign monoclonal gammopathy (a possible forerunner of multiple myeloma), and a first degree kinship with persons having a cancer or precancerous disease showing a Mendelian (genetic) inheritance pattern, e.g., familial polyposis of the colon, ulcerative colitis, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; Robbins and Angell, 1976, Basic Pathology, 2d Ed., W.B. Saunders Co., Philadelphia, pp. 112-113, etc.

In another specific embodiment, a Therapeutic of the invention is administered to a human patient to prevent progression to breast, colon, lung, pancreatic, prostatic, ovarian, stomach, or uterine cancer, or melanoma or sarcoma.

5.4.3. Hyperproliferative and Dysproliferative Disorders

In another embodiment of the present invention, a Therapeutic is administered to treat or prevent hyperproliferative or benign dysproliferative disorders. Therapeutics of the invention can be assayed by any method known in the art for efficacy in treating or preventing hyperproliferative diseases or disorders. Such assays include in vitro cell proliferation assays, in vitro or in vivo assays using animal models of hyperproliferative diseases or disorders, or any of the assays described in Sections 5.5 and 5.6 infra. Potentially effective Therapeutics, for example but not by way of limitation, decrease cell proliferation in culture or inhibit growth or cell proliferation in animal models in comparison to controls.

Accordingly, once a hyperproliferative disorder has been shown to be amenable to treatment by modulation of p27(Kip1):FKBP-12 complex activity, that hyperproliferative disease or disorder can be treated or prevented by administration of a Therapeutic that modulates p27(Kip1):FKBP-12 complex formation including supplying a p27(Kip1):FKBP-12 complex or the individual binding partners of a p27(Kip1):FKBP-12 complex. Specific embodiments are directed to treatment or prevention of cirrhosis of the liver (a condition in which scarring has overtaken normal liver regeneration processes), treatment of keloid (hypertrophic scar) formation (disfiguring of the skin in which the scarring process interferes with normal renewal), psoriasis (a common skin condition characterized by excessive proliferation of the skin and delay in proper cell fate determination), benign tumors, fibrocystic conditions, and tissue hypertrophy (e.g., prostatic hyperplasia).

5.4.4. Neurodegenerative Disorders

p27(Kip1) and its binding partner FKBP-12 have been implicated in the deregulation of cellular maturation and apoptosis, which are both characteristic of neurodegenerative disease. Accordingly, Therapeutics of the invention, particularly but not limited to those that modulate (or supply) p27(Kip1):FKBP-12 complex activity may be effective in treating or preventing neurodegenerative disease. Therapeutics of the present invention that modulate p27(Kip1):FKBP-12 complex activity involved in neurodegenerative disorders can be assayed by any method known in the art for efficacy in treating or preventing such neurodegenerative diseases and disorders. Such assays include in vitro assays for regulated cell maturation or inhibition of apoptosis or in vivo assays using animal models of neurodegenerative diseases or disorders, or any of the assays described in Sections 5.5, 5.6, and 5.8 infra. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation of p27(Kip1):FKBP-12 complex activity, that neurodegenerative disease or disorder can be treated or prevented by administration of a Therapeutic that modulates p27(Kip1):FKBP-12 complex formation or activity, including supplying a p27(Kip1):FKBP-12 complex or the individual binding partners p27(Kip1) and/or FKBP-12.

Such diseases include all degenerative disorders involved with aging, especially osteoarthritis and neurodegenerative disorders. Neurodegenerative disorders that can be treated or prevented include but are not limited to those listed in Table 2. Beal et al., 1997, In: Isslebacher et al., Eds., Harrison's Principals of Internal Medicine, 13^{th} Ed., McGraw Hill, New York, p. 2270, Table 370-371.

TABLE 2
NEURODEGENERATIVE DISORDERS

Progressive dementia in the absence of other neurological
signs:

Alzheimer's Disease (AD) (or early-onset AD)
Senile dementia of the Alzheimer's type (or late
onset AD)
Pick's Disease

Syndromes combining progressive dementia with prominent
neurological abnormalities:

Huntington's disease
Multiple system atrophy (dementia combined with
ataxia, Parkinson's disease, etc.)
Progressive supranuclear palsy
Diffuse Lewy body disease
Corticodentatonigral degeneration
Hallervorden-Spatz disease
Progressive familial myoclonic epilepsy

Syndromes of gradually developing abnormalities of posture
and movement:

Parkinson's disease
Striatonigral degeneration
Progressive supranuclear palsy
Torsion dystonia
Spasmodic torticollis and other restricted
dyskinesias
Familial tremor
Gilles de la Tourette syndrome

Syndromes of progressive ataxia:

Cerebellar cortical degeneration
Olivopontocerebellar atrophy
Friedreich's ataxia and related spinocerebellar
degenerations
Shy-Drager syndrome
Subacute necrotizing encephalopathy

Motor neuron disease without sensory changes:

Amyotrophic lateral sclerosis
Infantile spinal muscular atrophy
Juvenile spinal muscular atrophy
Other forms of familial spinal muscular atrophy
Primary lateral sclerosis
Hereditary spastic paraplegia

Motor neuron disease with sensory changes:

Peroneal muscular atrophy
Hypertrophic interstitial polyneuropathy
Other forms of chronic progressive neuropathy

Syndromes of progressive visual loss:
Retinitis pigmentosa

5.4.5. Autoimmune Disorders

FKBP-12 has been implicated in autoimmune disorders. Therapeutics of the invention, particularly those that modulate (or supply) p27(Kip1):FKBP-12 complex activity may be effective in treating or preventing autoimmune diseases or disorders. Therapeutics of the invention (particularly Therapeutics that modulate the levels or activity of FKBP-12) can be assayed by any method known in the art for efficacy in treating or preventing such autoimmune diseases and disorders, such assays include in vitro assays for using cell culture models as described in Section 5.5, infra, or in vivo assays using animal models of autoimmune diseases or disorders as described in Section 5.8 infra. Potentially effective Therapeutics, for example but not by way of limitation, reduce autoimmune responses in animal models in comparison to controls.

Accordingly, once an autoimmune disease or disorder has been shown to be amenable to treatment by modulation of p27(Kip1):FKBP-12 complex activity, that autoimmune disease or disorder can be treated or prevented by administration of a Therapeutic that modulates p27(Kip1):FKBP-12 complex formation including supplying a p27(Kip1):FKBP-12 complex or individual p27(Kip1) and/or FKBP-12 proteins.

Autoimmune disorders that can be treated or prevented include but are not limited to those listed in Table 3. Haynes and Fauci, 1997, In: Isslebacher et al., Ed., Harrison's Principals of Internal Medicine, 13^{th} Ed., McGraw Hill, New York, pp.1544-1545, Table 277-1.

TABLE 3
AUTOIMMUNE DISEASES

Organ Specific

Endocrine

Thyroid

Hashimoto's thyroiditis
Grave's disease
thyroiditis with hyperthyroidism

type I autoimmune polyglandular syndrome
type II autoimmune polyglandular syndrome
insulin-dependent diabetes mellitus
immune-mediated infertility
autoimmune Addison's disease

Epidermal

pemphigus vulgaris
pemphigus foliaceous
bullus pemphigoid
dermatitis herpetiformis
linear IgA dermatosis
epidermolysis bullous aquisita
autoimmune alopecia
erythema nodosa
contact dermatitis
herpes gestationis
cicatricial pemphigoid
chronic bullous disease of childhood

Hemotologic

autoimmune hemolytic anemia
lymphomas

chronic lymphocytic anemia
non-Hodgkin's lymphoma
Hodgkin's disease

drug-induced

alpha methyl dopa
penicillin type
quinidine type

post-viral infections
tumors (rare)
cold agglutinin diseases

acute

mycoplasma infection
infectious mononucleosis

chronic

idiopathic lymphoma

paroxysmal cold hemoglobinuria
autoimmune thrombocytopenic purpura

idiopathic
drug-induced

autoimmune neutropenia

Neuromuscular

myasthenia gravis
acute disseminated encephalomyelitis
multiple sclerosis
Guillain-Barre syndrome
Chronic inflammatory demyelinating
polyradiculoneuropathy

Hepatobiliary

autoimmune chronic active hepatitis
primary biliary sclerosis
sclerosing cholangitis

Gastrointestinal

gluten-sensitive enteropathy
pernicious anemia
inflammatory bowel disease

Non-Organ Specific

Connective tissue disease

system lupus erythematososis
rheumatoid arthritis
scleroderma
mixed connective tissue disease
psoriasis
polymyositis
dermatomyositis
Sjogren's syndrome
ankylosing spondylitis
reactive arthritis
undifferentiated spondylarthropathy
Behcet's syndrome

Vasculitis syndromes

systemic necrotizing vasculitides

classic polyarteritis nodosa
Churg-Strauss disease
polyangiitis overlap syndrome

hypersensitivity vasculitis
Wegener's granulomatosis
temporal arteritis
Takayasu's arteritis

Kawasaki's disease
isolated vasculitis of the central nervous system
thromboangiitis obliterans

Sarcoidosis
Graft-vs-host disease

5.4.6. Disorders Related to Organ Transplantation

FKBP-12 has been implicated in disorders related to organ transplantation, in particular but not limited to organ rejection. Therapeutics of the invention, particularly those that modulate (or supply) p27(Kip1):FKBP-12 complex activity, may be effective in treating or preventing diseases or disorders related to organ transplantation. Therapeutics of the invention (particularly Therapeutics that modulate the levels or activity of a p27(Kip1):FKBP-12 complex) can be assayed by any method known in the art for efficacy in treating or preventing such diseases and disorders related to organ transplantation, such assays include in vitro assays for using cell culture models as described in Section 5.8, infra, or in vivo assays using animal models of diseases and disorders related to organ transplantation as described in Section 5.5 infra. Potentially effective Therapeutics, for example but not by way of limitation, reduce immune rejection responses in animal models in comparison to controls.

Accordingly, once diseases and disorders related to organ transplantation are shown to be amenable to treatment by modulation of p27(Kip1):FKBP-12 complex activity or formation, such diseases or disorders can be treated or prevented by administration of a Therapeutic that modulates p27(Kip1):FKBP-12 complex activity or formation (including supplying a p27(Kip1):FKBP-12 complex or individual p27(Kip1) and/or FKBP-12 proteins).

5.4.7. Cardiovascular Disease

p27(Kip1) has been implicated in cardiovascular disorders, including in atherosclerotic plaque formation. All diseases shown in Table 4 below, are either directly or indirectly, associated with atherosclerosis. Accordingly, Therapeutics of the invention, particularly those that modulate (or supply) p27(Kip1):FKBP-12 complex activity or formation, may be effective in treating or preventing atherosclerosis-associated diseases or disorders. Therapeutics of the invention (particularly Therapeutics that modulate the levels or activity of a p27(Kip1):FKBP-12 complex) can be assayed by any method known in the art, including those described in Section 5.5 and 5.6, infra, for efficacy in treating or preventing such diseases and disorders.

A vast array of animal and cell culture models exist for processes involved in atherosclerosis. A limited and non-exclusive list of animal models includes knockout mice for premature atherosclerosis (Kurabayashi and Yazaki, 1996, Int. Angiol. 15:187-194), transgenic mouse models of atherosclerosis (Kappel et al., 1994, FASEB J. 8:583-592), antisense oligonucleotide treatment of animal models (Callow, 1995, Curr. Opin. Cardiol. 10:569-576), transgenic rabbit models for atherosclerosis (Taylor, 1997, Ann. N.Y. Acad. Sci 811:146-152), hypercholesterolemic animal models (Rosenfeld, 1996, Diabetes Res. Clin. Pract. 30 Suppl.:1-11), hyperlipidemic mice (Paigen et al., 1994, Curr. Opin. Lipidol. 5:258-264), and inhibition of lipoxygenase in animals (Sigal et al., 1994, Ann. N.Y. Acad. Sci. 714:211-224). In addition, in vitro cell models include but are not limited to monocytes exposed to low density lipoprotein (Frostegard et al., 1996, Atherosclerosis 121:93-103), cloned vascular smooth muscle cells (Suttles et al., 1995, Exp. Cell Res. 218:331-338), endothelial cell-derived chemoattractant exposed T cells (Katz et al., 1994, J. Leukoc. Biol. 55:567-573), cultured human aortic endothelial cells (Farber et al., 1992, Am. J. Physiol. 262:H1088-1085), and foam cell cultures (Libby et al,, 1996, Curr Opin Lipidol 7:330-335). Potentially effective Therapeutics, for example but not by way of limitation, reduce foam cell formation in cell culture models, or reduce atherosclerotic plaque formation in hypercholesterolemic mouse models of atherosclerosis in comparison to controls.

Accordingly, once an atherosclerosis-associated disease or disorder has been shown to be amenable to treatment by modulation of p27(Kip1):FKBP-12 complex activity or formation, that disease or disorder can be treated or prevented by administration of a Therapeutic that modulates p27(Kip1):FKBP-12 complex activity or formation including supplying a p27(Kip1):FKBP-12 complex, or individual p27(Kip1) and/or FKBP-12 proteins.

Diseases associated with atherosclerosis that can be treated or prevented include but are not limited to those listed in Table 4. Bierman, 1997, In: Isslebacher et al., Ed., Harrison's Principals of Internal Medicine, 13th Ed., McGraw Hill, New York, p. 1107, Table 208-2.

TABLE 4
DISEASES ASSOCIATED WITH ATHEROSCLEROSIS

Cardiovascular disease

cerebral thrombosis
cerebral hemorrhage
ischemic heart disease
peripheral vascular disease
ischemic renal disease
thrombosis of other major vessels

Other

diabetes mellitus
hypertension
familial hypercholesterolemia
familial combined hyperlipidemia
familial dysbetalipoproteinemia
familial hypoalphalipoproteinemia
hypothyroidism
cholesterol ester storage disease
systemic lupus erythematosus
homocysteinemia

5.4.8. Gene Therapy

In a specific embodiment, nucleic acid molecules comprising a sequence encoding a p27(Kip1) and/or FKBP-12, or a functional derivative thereof, are administered to modulate p27(Kip1):FKBP-12 complex activity or formation, by way of gene therapy. In more specific embodiments, a nucleic acid molecule or nucleic acid molecules encoding both p27(Kip1) and FKBP-12, or functional derivatives thereof, are administered by way of gene therapy. Gene therapy refers to therapy performed by the administration of a nucleic acid molecule to a subject. In this embodiment of the present invention, a nucleic acid molecule produces its encoded protein(s) and mediates a therapeutic effect by modulating p27(Kip1):FKBP-12 complex activity or formation. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; and May, 1993, TIBTECH 11:155-215). Methods commonly known in the art of recombinant DNA technology which can be used are described in Ausubel et al., eds., 1993, Current Protocols in Molecular Biology, John Wiley & Sons, NY; and Kriegler, 1990, Gene Transfer and Expression, A Laboratory Manual, Stockton Press, NY.

In a preferred aspect, the Therapeutic comprises a p27(Kip1) and FKBP-12 encoding nucleic acid molecule that is part of an expression vector that expresses the proteins p27(Kip1) and FKBP-12 or fragments or chimeric proteins thereof in a suitable host. In particular, such a nucleic acid molecule has a promoter operably linked to the p27(Kip1) and the FKBP-12 coding region(s) (or, less preferably two separate promoters linked to the p27(Kip1) and the FKBP-12 coding regions, respectively, separately), the promoter being inducible or constitutive, and, optionally, tissue-specific. In another particular embodiment, a nucleic acid molecule is used in which the p27(Kip1) and FKBP-12 coding sequences and any other desired sequences, are flanked by regions that promote homologous recombination at a desired site in the genome, thus providing for intra-chromosomal expression of the p27(Kip1) and/or the FKBP-12 nucleic acid molecules. Koller and Smithies, 1989, Proc. Natl. Acad. Sci. USA 86:8932-8935; Zijlstra et al., 1989, Nature 342:435-438.

Delivery of the nucleic acid molecule into a patient may be either direct, in which case the patient is directly exposed to the nucleic acid molecule or nucleic acid molecule-carrying vector, or indirect, in which case, cells are first transformed with the nucleic acid molecule in vitro, then transplanted into the patient. These two approaches are known, respectively, as in vivo or ex vivo gene therapy.

In a specific embodiment, the nucleic acid molecule is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (e.g., Wu and Wu, 1987, J. Biol. Chem. 262:4429-4432) (which can be used to target cell types specifically expressing the receptors), etc. In another embodiment, a nucleic acid molecule-ligand complex can be formed in which the ligand comprises a fusogenic viral peptide to disrupt endosomes, allowing the nucleic acid molecule to avoid lysosomal degradation. In yet another embodiment, the nucleic acid molecule can be targeted in vivo for cell specific uptake and expression, by targeting a specific receptor (see, e.g., Wu et al., 1992, International Patent Publication WO 92/06180; Wilson et al., 1992, International Patent Publication WO 92/22635; Findeis et al., 1992, International Patent Publication WO 92/20316); Clark et al., 1993, International Patent Publication Wo 93/14188; and Young, 1993, International Patent Publication WO 93/20221). Alternatively, the nucleic acid molecule can be introduced intracellularly and incorporated within host cell DNA for expression, by homologous recombination. Koller and Smithies, 1989, Proc. Natl. Acad. Sci. USA 86:8932-8935; Zijlstra et al., 1989, Nature 342:435-438.

In a specific embodiment, a viral vector that contains the p27(Kip1) and/or the FKBP-12 encoding nucleic acid molecule(s) is used. For example, a retroviral vector can be used. Miller et al., 1993, Meth. Enzymol. 217:581-599. These retroviral vectors have been modified to delete retroviral sequences that are not necessary for packaging of the viral genome and integration into host cell DNA. The p27(Kip1) and/or FKBP-12 (preferably both p27(Kip1) and FKBP-12) encoding nucleic acid molecule(s) to be used in gene therapy is/are cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. See, Kozarsky and Wilson, 1993, Current Opinion in Genetics and Development 3:499-503, a review which discusses adenovirus-based gene therapy. The use of adenovirus vectors to transfer genes to the respiratory epithelia of rhesus monkeys has been demonstrated. Bout et al., 1994, Human Gene Therapy 5:3-10. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy. Walsh et al., 1993, Proc. Soc. Exp. Biol. Med. 204:289-300.

Another approach to gene therapy involves transferring a gene into cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene from these that have not. Those cells are then delivered to a patient.

In this embodiment, the nucleic acid molecule is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; and Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid molecule to the cell, so that the nucleic acid sequence is expressed by the cell and preferably, is heritable and expressed by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid molecule can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient. In another embodiment in which recombinant cells are used in gene therapy, a p27(Kip1) and/or FKBP-12 (preferably both a p27(Kip1) and FKBP-12) encoding nucleic acid molecule is/are introduced into the cells such that the gene or genes are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (PCT Publication Wo 94/08598, dated Apr. 28, 1994), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 2A: 229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture. Rheinwald, 1980, Meth. Cell Bio. 2A:229; Pittelkow and Scott, 1986, Mayo Clinic Proc. 61:771. If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration, see, e.g., Kodo et al., 1984, J. Clin. Invest. 73:1377-1384. In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid molecule to be introduced for purposes of gene therapy comprises an inducible promoter operably linked to the coding region, such that expression of the nucleic acid is controllable by controlling the presence or absence of the appropriate inducer of transcription.

Additional methods can be adapted for use to deliver a nucleic acid encoding the p27(Kip1) and/or FKBP-12 proteins, or functional derivatives thereof, e.g., as described in Section 5.1, supra.

5.4.9. Use of Antisense Oligonucleotides For Suppression of p27(KIPl):FKBP-12 Complex Activity or Formation

In a specific embodiment, p27(Kip1):FKBP-12 complex activity or formation is inhibited by use of antisense nucleic acid molecules for p27(Kip1) and/or FKBP-12 (preferably both p27(Kip1) and FKBP-12). The present invention provides the therapeutic or prophylactic use of nucleic acid molecule of at least six nucleotides that are antisense to a gene or cDNA encoding p27(Kip1) and/or FKBP-12 or portions thereof. A p27(Kip1) or FKBP-12 “antisense” nucleic acid molecule as used herein refers to a nucleic acid molecule capable of hybridizing to a portion of a p27(Kip1) or FKBP-12 RNA (preferably mRNA) by virtue of some sequence complementarily. The antisense nucleic acid molecule may be complementary to a coding and/or noncoding region of a p27(Kip1) or FKBP-12 mRNA. Such antisense nucleic acid molecules that inhibit p27(Kip1):FKBP-12 complex activity or formation have utility as Therapeutics, and can be used in the treatment or prevention of disorders as described, supra.

The antisense nucleic acid molecules of the present invention can be oligonucleotides that are double-stranded or single-stranded, RNA or DNA, or a modification or derivative thereof, which can be directly administered to a cell, or which can be produced intracellularly by transcription of exogenous, introduced sequences.

In another embodiment, the present invention is directed to methods for inhibiting the expression of p27(Kip1) and/or FKBP-12 nucleic acid sequences, in a prokaryotic or eukaryotic cell, comprising providing the cell with an effective amount of a composition comprising an antisense nucleic acid molecule of p27(Kip1) and FKBP-12, or derivatives thereof, of the present invention.

The p27(Kip1) and FKBP-12 antisense nucleic acid molecules are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; International Patent Publication WO 88/09810) or blood-brain barrier (see, e.g., International Patent Publication WO 89/10134, hybridization-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, a p27(Kip1) and/or FKBP-12 antisense oligonucleotide is provided, referably as single-stranded DNA. The oligonucleotide may be modified at any position on its structure with constituents generally known in the art.

The p27(Kip1) and FKBP-12 antisense oligonucleotides may comprise at least one modified base oiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-a-anomeric oligonucleotide. An a-anomeric oligonucleotide forms specific double-stranded hybrids with complementary RNA in which, contrary to the usual β-units, the strands run parallel to each other. Gautier et al., 1987, Nucl. Acids Res. 15:6625-6641.

The oligonucleotide may be conjugated to another molecule, e.g., a peptide, hybridization-triggered cross-linking agent, transport agent, hybridization-triggered cleavage agent, etc.

Oligonucleotides of the invention may be synthesized by standard methods known in the art, e.g., by use of an automated DNA synthesizer. As examples, phosphorothioate oligonucleotides may be synthesized by the method of Stein et al., 1988, Nucl. Acids Res. 16:3209, methylphosphonate oligonucleotides can be prepared by use of controlled pore glass polymer supports (Sarin et al., 1988, Proc. Natl. Acad. Sci. U.S.A. 85:7448-7451), etc.

In a specific embodiment, the p27(Kip1) and FKBP-12 antisense oligonucleotides comprise catalytic RNAs, or ribozymes (see, e.g., PCT International Publication Wo 90/11364, published Oct. 4, 1990; Sarver et al., 1990, Science 247:1222-1225). In another embodiment, the oligonucleotide is a 2′-O-methylribonucleotide (Inoue et al., 1987, Nucl. Acids Res. 15:6131-6148), or a chimeric RNA-DNA analog (Inoue et al., 1987, FEBS Lett. 215:327-330).

In an alternative embodiment, the p27(Kip1) and FKBP-12 antisense nucleic acids of the invention are produced intracellularly by transcription from an exogenous sequence. For example, a vector can be introduced in vivo such that it is taken up by a cell, within which cell the vector-or a portion thereof is transcribed, producing an antisense nucleic acid (RNA) of the invention. Such a vector would contain a sequence encoding p27(Kip1) and FKBP-12 (preferably, a p27(Kip1) and FKBP-12 antisense nucleic acid molecule). Such a vector can remain episomal or become chromosomally integrated, as long as it can be transcribed to, produce the desired antisense RNA. Such vectors can be constructed by recombinant DNA technology methods standard in the art. Vectors can be plasmid, viral, or others known in the art, to be capable of replication and expression in mammalian cells. Expression of the sequences encoding the p27(Kip1) and FKBP-12 antisense RNAs can be by any promoter known in the art to act in mammalian cells, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids of the invention comprise a sequence complementary to at least a portion of an RNA transcript of a p27(Kip1) or a FKBP-12 gene, preferably a human p27(Kip1) or FKBP-12 gene. However, absolute complementarily, although preferred, is not required. A sequence “complementary to at least a portion of an RNA”, as referred to herein, means a sequence having sufficient complementarily to be able to hybridize with the RNA, forming a stable duplex; in the case of double-stranded p27(Kip1) or FKBP-12 antisense nucleic acid molecules, a single strand of the duplex DNA may thus be tested, or triplex formation may be assayed. The ability to hybridize will depend on both the degree of complementarily and the length of the antisense nucleic acid. Generally, the longer the hybridizing nucleic acid, the more base mismatches with a p27(Kip1) or FKBP-12 RNA it may contain and still form a stable duplex or triplex, as the case may be. One skilled in the art can ascertain a tolerable degree of mismatch by use of standard procedures to determine the melting point of the hybridized complex.

The p27(Kip1) and FKBP-12 antisense nucleic acid molecule can be used to treat (or prevent) disorders of a cell type that expresses, or overexpresses a p27(Kip1):FKBP-12 complex. In a preferred embodiment, a single-stranded DNA antisense p27(Kip1) and/or FKBP-12 antisense oligonucleotide or single-stranded DNA antisense p27(Kip1):FKBP-12 fusion is used.

Cell types that express or overexpress p27(Kip1) and/or FKBP-12 RNA can be identified by various methods known in the art. Such methods include, but are not limited to, hybridization with p27(Kip1)- and FKBP-12-specific nucleic acid molecules, e.g. by Northern hybridization, dot blot hybridization, in situ hybridization, or by observing the ability of RNA from the cell type to be translated in vitro into p27(Kip1) or FKBP-12 by immunohistochemistry. In a preferred aspect, primary tissue from a patient can be assayed for p27(Kip1) and/or FKBP-12 expression prior to treatment, e.g., by immunocytochemistry, in situ hybridization, or any number of methods to detect protein or mRNA expression.

Pharmaceutical compositions of the invention (see Section 5.7, infra), comprising an effective amount of a p27(Kip1) and/or FKBP-12 antisense nucleic acid in a pharmaceutically acceptable carrier can be administered to a patient having a disease or disorder which is of a type that expresses or overexpresses a p27(Kip1):FKBP-12 complex.

The amount of p27(Kip1) and/or FKBP-12 antisense nucleic acid that will be effective in the treatment of a particular disorder or condition will depend on the nature of the disorder or condition, and can be determined by standard clinical techniques. Where possible, it is desirable to determine the antisense cytotoxicity in vitro, and then in useful animal model systems, prior to testing and use in humans.

In a specific embodiment, pharmaceutical compositions comprising p27(Kip1) and FKBP-12 antisense nucleic acid molecules are administered via liposomes, microparticles, or microcapsules. In various embodiments of the invention, it may be useful to use such compositions to achieve sustained release of the p27(Kip1) and/or FKBP-12 antisense nucleic acid molecules. In a specific embodiment, it may be desirable to utilize liposomes targeted via antibodies to specific identifiable central nervous system cell types. Leonetti et al., 1990, Proc. Natl. Acad. Sci. U.S.A. 87:2448-2451; Renneisen et al., 1990, J. Biol. Chem. 265:16337-16342.

5.5. Assays of p27(Kip1):FKBP-12 Complexes and Derivatives and Analogs Thereof

The functional activity of a p27(Kip1):FKBP-12 complex or of a derivative, fragment or analog thereof can be assayed by various methods. Potential modulators (e.g., agonists and antagonists) of p27(Kip1):FKBP-12 complex activity or formation, e.g., anti-p27(Kip1):FKBP-12 antibodies, or p27(Kip1) or FKBP-12 antisense nucleic acids, can be assayed for the ability to modulate p27(Kip1):FKBP-12 complex activity or formation.

In one embodiment of the present invention, where one is assaying for the ability to bind or compete with a wild-type p27(Kip1):FKBP-12 complex for binding to an anti-p27(Kip1):FKBP-12 antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention.

The expression of p27(Kip1) and FKBP-12 genes (both endogenous genes and those expressed from cloned DNA containing these genes) can be detected using techniques known in the art, including but not limited to Southern hybridization (Southern, 1975, J. Mol. Biol. 98:503-517), northern hybridization (see, e.g., Freeman et al., 1983, Proc. Natl. Acad. Sci. USA 80:4094-4098), restriction endonuclease mapping (Sambrook et al., 1989, Molecular Cloning, A Laboratory Manual, 2^{nd} Ed. Cold Spring Harbor Laboratory Press, New York), RNase protection (Current Protocols in Molecular Biology, John Wiley and Sons, New York, 1997), DNA sequence analysis, and polymerase chain reaction amplification (PCR) (U.S. Pat. Nos. 4,683,202, 4,683,195, and 4,889,818; Gyllenstein et al., 1988, Proc. Natl. Acad. Sci. USA 85:7652-7657; Ochman et al., 1988, Genetics 120:621-623; Loh et al., 1989, Science 243:217-220) followed by Southern hybridization or with probes specific for p27(Kip1) or FKBP-12 genes, in various cell types. Methods of amplification other than PCR commonly known in the art also can be employed. In one embodiment, Southern hybridization can be used to detect genetic linkage of p27(Kip1) and FKBP-12 gene mutations to physiological or pathological states. Various cell types, at various stages of development, can be characterized for their expression of p27(Kip1) and FKBP-12 (particularly expression of p27(Kip1) and FKBP-12 at the same time and in the same cells). The stringency of the hybridization conditions for northern or Southern blot analysis can be manipulated to ensure-detection of nucleic acids with the desired degree of relatedness to the specific probes used. Modifications to these methods and other methods commonly known in the art can be used.

Derivatives (e.g., fragments), homologs and analogs of FKBP-12 can be assayed for binding to p27(Kip1) by any method known in the art, including, for example, the modified yeast matrix mating test described in Section 5.6.1 infra, immunoprecipitation with an antibody that binds to p27(Kip1) in a complex followed by analysis by size fractionation of the immunoprecipitated proteins (e.g. by denaturing or nondenaturing polyacrylamide gel electrophoresis), Western analysis, non-denaturing gel electrophoresis, etc.

One embodiment of the invention provides a method for screening a derivative, homolog or analog of p27(Kip1) for biological activity comprising contacting said derivative, homolog or analog of p27(Kip1) with FKBP-12 and detecting the formation of a complex between said derivative, homolog or analog of p27(Kip1) and FKBP-12; wherein detecting formation of said complex indicates that said derivative, homolog or analog of p27(Kip1) has biological, e.g., binding, activity. Additionally, another embodiment of the invention relates to a method for screening a derivative, homolog or analog of FKBP-12 for biological activity comprising contacting said derivative, homolog or analog of FKBP-12 with p27(Kip1) and detecting the formation of a complex between said derivative, homolog or analog of FKBP-12 and p27(Kip1); wherein detecting the formation of said complex indicates that said derivative, homolog or analog of said protein has biological activity.

The present invention also provides methods of modulating the activity of a protein that can participate in a p27(Kip1):FKBP-12 complex, e.g., p27(Kip1) and FKBP-12, by administration of a binding partner of said protein or derivative, homolog or analog thereof. p27(Kip1) and derivatives and analogs thereof, can be assayed for the ability to modulate the activity or levels of FKBP-12 by contacting a cell with or administering to an animal expressing a FKBP-12 gene.a p27(Kip1) protein, derivative or analog, or a nucleic acid encoding a p27(Kip1) protein, derivative or analog, or an antibody that immunospecifically binds the p27(Kip1) protein or a fragment or derivative of said antibody containing the binding domain thereof and measuring a change in FKBP-12 levels or activity, wherein a change in FKBP-12 levels or activity indicates that the p27(Kip1) protein, derivative or analog can modulate FKBP-12 levels or activity. Alternatively, FKBP-12 can be assayed for the ability to modulate the activity or levels of a p27(Kip1) protein by contacting a cell with or administering to an animal expressing a p27(Kip1) protein a FKBP-12 protein, derivative or analog, or a nucleic acid encoding a FKBP-12 protein, derivative or analog, or an antibody that immunospecifically binds FKBP-12, or a fragment or derivative of said antibody containing the binding domain thereof, wherein a change in p27(Kip1) levels or activity indicates that the FKBP-12 can modulate p27(Kip1) levels or activity.

p27(Kip1) and its identified binding partner FKBP-12, have roles in the control of cell proliferation and, therefore, cell-transformation and tumorigenesis. Accordingly, methods of the invention are provided for screening a p27(Kip1):FKBP-12 complex or a fragment, derivative or analog of the complex, for activity in altering cell proliferation, cell transformation and/or tumorigenesis in vitro and in vivo.

The p27(Kip1):FKBP-12 complex or derivative, fragment, or analog thereof, can be assayed for activity to alter (i.e., increase or decrease) cell proliferation in cultured cells in vitro using methods that are well known in the art for measuring cell proliferation. Specific examples of cell culture models include, but are not limited to: for lung cancer, primary rat lung tumor cells (Swafford et al., 1997, Mol. Cell. Biol., 17:1366-1374) and large-cell undifferentiated cancer cell lines (Mabry et al., 1991, Cancer Cells, 3:53-58); colorectal cell lines for colon cancer (Park and Gazdar, 1996, J. Cell Biochem. Suppl. 24:131-141); multiple established cell lines for breast cancer (Hambly et al., 1997, Breast Cancer Res. Treat. 43:247-258; Gierthy et al., 1997, Chemosphere 34:1495-1505; 35 Prasad and Chiurch, 1997, Biochem. Biophys. Res. Commun. 232:14-19); a number of well-characterized cell models for prostate cancer (Webber et al., 1996, Prostate, Part 1, 29:386-394; Parts 2 and 3, 30:58-64 and 136-142; Boulikas, 1997, Anticancer Res. 17:1471-1505); for genitourinary cancers continuous human bladder cancer cell lines (Ribeiro et al., 1997, Int. J. Radiat. Biol. 72:11-20), organ cultures of transitional cell carcinomas (Booth et al., 1997, Lab Invest. 76:843-857), and rat progression models (Vet et al., 1997, Biochem. Biophys. Acta 1360:39-44); and established cell lines for leukemias and lymphomas (Drexler, 1994, Leuk. Res. 18:919-927; Tohyama, 1997, Int. J. Hematol. 65:309-317).

For example, but not by way of limitation, cell proliferation can also be assayed by measuring ³H-thymidine incorporation, by direct cell count, by detecting changes in transcriptional activity of known genes such as proto-oncogenes (e.g., fos, myc) or cell cycle markers, etc. Accordingly, one embodiment of the present invention provides a method for screening a p27(Kip1):FKBP-12 complex or a fragment, derivative, or analog thereof, for activity in altering (i.e., increasing or decreasing) proliferation of a cell in vitro, comprising contacting the cell with a p27(Kip1):FKBP-12 complex or derivative, analog, or fragment thereof, measuring the proliferation of the cell that has been so contacted, and comparing the proliferation of the cell so contacted with the complex or derivative, analog or fragment with the proliferation of a cell not so contacted with the complex or derivative, analog or fragment, wherein a change in the level of proliferation in said contacted cell as compared to the non-contacted cell indicates that the complex or derivative, analog or fragment has activity to alter cell proliferation.

A p27(Kip1):FKBP-12 complex or derivative, fragment or analog thereof, can also be screened for activity in inducing or inhibiting cell transformation (or progression to malignant phenotype) in vitro. The complexes and proteins of the invention can be screened by contacting either cells with a normal phenotype (for assaying cell transformation) or a transformed cell phenotype (for assaying inhibition of cell transformation) with a complex or protein of the present invention, and examining the cells for the acquisition or loss of characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, expression of fetal antigens, disappearance of the 250 kD surface protein, etc. See, generally, Luria et al., 1978, General Virology, 3d Ed., John Wiley & Sons, New York, pp. 436-446.

A p27(Kip1):FKBP-12 complex or a derivative, fragment, or analog thereof can also be screened for activity to promote or inhibit tumor formation in vivo in non-human test animals. A vast number of animal models of hyperproliferative disorders, including tumorigenesis and metastatic spread, are known in the art (see Mendelson, 1997, In: Harrison's Principals of Internal Medicine, 13th Edition, Isselbacher et al., Eds., McGraw-Hill, New York, p.1816, Table 317-2; Lovejoy et al., 1997, J. Pathol. 181:130-135). Specific examples include: for lung cancers, transplantation of tumor modules into rats (Wang et al., 1997, Ann. Thorac. Surg. 64:216-219) or establishment of lung cancer metastases in SCID mice depleted of NK cells (Yono and Sone, 1997, Gan To Kagaku Ryoho, 24:489-494); colon cancer transplantation of human colon cancer cells into nude mice (Gutman and Fidler, 1995, World J. Surg. 19:226-234), the cotton top tamarin model of human ulcerative colitis (Warren, 1996, Aliment. Pharmacol. Ther. 10 Suppl2:45-47) and mouse models with mutations of the adenomatous polyposis coli tumor suppressor (Polakis, 1997, Biochim. Biophys. Acta 1332:F127-F147); for breast cancer, transgenic models of breast cancer (Dankort and Muller, 1996, Cancer Treat. Res. 83:71-88; Amundadittir et al., 1996, Breast Cancer Res. Treat. 39:119-135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35-40); for genitourinary cancers, induced bladder neoplasm in rats and mice (Oyasu, 1995, Food Chem. Toxicol 33:747-755) and xenografts of human transitional cell carcinomas into nude rats (Jarrett et al., 1995, J. Endourol. 9:1-7); for hematopoietic cancers, transplanted allogeneic marrow in animals (Appelbaum, 1997, Leukemia 11 Suppl 4:S15-S17). Further, general animal models applicable to many types of cancer have been described, including but not restricted to the p53-deficient mouse model (Donehower, 1996, Semin. Cancer Biol. 7:269-278), the Min mouse (Shoemaker et al., 1997, Biochem. Biophys. Acta, 1332:F25-F48), and immune response to tumors in the rat (Frey, 1997, Methods, 12:173-188).

For example, a complex or protein of the present invention can be administered to a non-human test animal (preferably a test animal predisposed to develop a type of tumor) and the non-human test animal subsequently examined for an increased incidence of tumor formation in comparison with controls not administered the complex or protein of the present invention, i.e., FKBP-12, p27(Kip1) or a complex of p27(Kip1) and FKBP-12. Alternatively, the complexes and proteins of the present invention can be administered to a non-human test animal having a tumor (e.g., an animal in which a tumor has been induced by introduction of malignant, neoplastic, or transformed cells, or by administration of a carcinogen) and subsequently examining the tumor in the test animal for tumor regression in comparison to a control animal.

Viral oncoproteins have been shown to directly affect the function of p27(Kip1) in TGF-beta treated cells by binding to p27(Kip1) and blocking its inhibitory effect on cell proliferation (Mal et al., 1996, Nature 380:262-265; Nomura et al., 1997, Gene 1991:211-218). By virtue of p27(Kip1) binding, the tumor enhancing activity of the viral oncoproteins may be suppressed. Accordingly, a complexe or protein of the present invention can be screened for the ability to modulate, i.e., increase or decrease, p27(Kip1) binding to a cyclin-CDK complex or for the ability to modulate the oncogenic activity of a viral oncoprotein such as the adenoviral E1A protein, e.g., by any protein binding assay known in the art. p27(Kip1) can influence other oncogenes, for example, but not limited to, c-myc and retinoblastoma protein. Scott et al., 1997, Curr. Top. Microbiol. Immunol. 224:103-112; Eyhevesky et al., 1996, Mol. Biol. Cell. 7:553-564.

A p27(Kip1):FKBP-12 complex or individual members of the complex, or a derivative, analog, or fragment of the complex or member of the complex, can also be screened for activity in modulating the activity of other p27(Kip1) binding partners, i.e., cyclin D-CDK4, cyclin E-CDK2, cyclin A-CDK2, CDC2, and adenoviral protein E1A. For example, CDK2 has been shown to bind p27(Kip1). Kwon et al., 1996, Biochem. Biophys. Res. Comm. 220:703-709. Accordingly, a complex or protein of the present invention can be screened for the ability to modulate, i.e., increase or decrease, CDK2 effects on the cell cycle. Similarly, CDK2 itself interacts with retinoblastoma protein, p53, the transcription factor E2F, histone H1, and other proteins central to cell cycle control. Higashi et al., 1996, Eur. J. Biochem. 237:460-467. Thus, a complex or protein of the present invention can be screened by assaying for changes in the level of p53 phosphorylation, retinoblastoma protein phosphorylation, etc., e.g., as described in Milne et al., 1994, J. Biol. Chem. 269:9253-9260, or the level of CDK2 binding to histone H1, e.g., by methods described, supra.

Accordingly, once a hyperproliferative disease or disorder has been shown to be amenable to treatment by modulation of p27(Kip1):FKBP-12 complex activity or formation that disease or disorder can be treated or prevented by administration of a Therapeutic that modulates p27(Kip1):FKBP-12 complex activity or formation, including supplying a p27(Kip1):FKBP-12 complex. In a specific embodiment, a p27(Kip1):FKBP-12 complex is administered.

Similarly, once a neurodegeneration disease or disorder has been shown to be amenable to treatment by modulation of p27(Kip1):FKBP-12 complex activity or formation that disease or disorder can be treated or prevented by administration of a Therapeutic that modulates p27(Kip1):FKBP-12 complex activity or formation, including supplying a p27(Kip1):FKBP-12 complex. In a specific embodiment, a p27(Kip1):FKBP-12 complex is administered to treat or prevent a neurodegenerative disease or disorder. p27(Kip1) has been implicated in the development and involution of all organs, including the central nervous system. Casaccia-Bonnefil et al., 1997, Genes and Dev. 11:2335-2346. Accordingly, a p27(Kip1):FKBP-12 complex or derivative, homolog, analog or fragment thereof, nucleic acid molecules encoding p27(Kip1) or FKBP-12, anti-p27(Kip1):FKBP-12 complex antibodies, and other modulators of p27(Kip1):FKBP-12 complex activity or formation can be tested for activity in treating or preventing neurodegenerative disease in in vitro and in vivo assays.

In one embodiment, a Therapeutic of the invention can be assayed for activity in treating or preventing neurodegenerative disease by contacting a cultured cell that exhibits an indicator of a neurodegenerative disease in vitro, with the Therapeutic and comparing the level of said indicator in the cell contacted with the Therapeutic, with said level of said indicator in a cell not so contacted, wherein a lower level in said contacted cell indicates that the Therapeutic has activity in treating or preventing neurodegenerative disease. Specific examples of such cultured models for neurodegenerative disease include, but are not limited to, cultured rat endothelial cells from affected and nonaffected individuals (Maneiro et al., 1997, Methods Find. Exp. Clin. Pharmacol. 19:5-12); P19 murine embryonal carcinoma cells (Hung et al., 1992, Proc. Natl. Acad. Sci. USA 89:9439-9443); and dissociated cell cultures of cholinergic neurons from nucleus basalis of Meynert (Nakajima et al., 1985, Proc. Natl. Acad. Sci. USA 82:6325-6329).

In another embodiment, a Therapeutic of the invention can be assayed for activity in treating or preventing neurodegenerative disease by administering the Therapeutic to a test animal that is predisposed to develop symptoms of a neurodegenerative disease, and measuring the change in said symptoms of the neurodegenerative disease after administration of said Therapeutic, wherein a reduction in the severity of the symptoms of the neurodegenerative disease or prevention of the symptoms of the neurodegenerative disease indicates that the Therapeutic has activity in treating or preventing neurodegenerative disease. Such a test animal can be any one of a number of animal models known in the art for neurodegenerative disease. These models, including those for Alzheimer's Disease and mental retardation of trisomy 21, accurately mimic natural human neurodegenerative diseases. Farine, 1997, Toxicol. 119:29-35. Examples of specific models include, but are not limited to, the partial trisomy 16 mouse (Holtzman et al., 1996, Proc. Natl. Acad. Sci. USA 93:13333-13338); bilateral nucleus basalis magnocellularis-lesioned rats (Popovic et al., 1996, Int. J. Neurosci. 86:281-299); the aged rat (Muir, 1997, Pharmacol. Biochem. Behav. 56:687-696); the PDAPP transgenic mouse model of Alzheimer's disease (Johnson-Wood et al., 1997, Proc. Natl. Acad. Sci. USA 94:1550-1555); and experimental autoimmune dementia (Oron et al., 1997, J. Neural Transm. Suppl. 49:77-84).

The immunosuppressants rapamycin and FK506 bind to FKBP-12 via an oval-shaped hydrophobic pocket on FKBP-12. Galat, 1996, Eur. J. Biochem. 216:689-707. The immunosuppressant rapamycin plays an important role in disrupting cytokine signaling that promotes lymphocyte growth and differentiation. In addition to its function as an immunosuppressant, rapamycin is a potent inhibitor of cellular proliferation. This antiproliferative effect is mediated through the formation of a complex with rapamycin and FKBP-12. Widerrecht et al., 1995, Prog. Cell Cycle Res. 1:53-71; Marx et al., 1995, Circ. Res. 76:412-417.

The immunosuppressant FK506 selectively interacts with and inhibits a calcium-dependent serine-threonine phosphatase function after binding to FKBP-12. This phosphatase modulates calcium release in skeletal and cardiac muscles. Thus, a complexe or protein of the present invention can be screened by measuring its effect on the levels of FKBP-12 binding to rapamycin or FK506, as well as on p27(Kip1):FKBP-12 complex formation or activity.

The p27(Kip1) binding partner FKBP-12 is implicated in autoimmune disease. Accordingly, a p27(Kip1):FKBP-12 complex or derivative, analog, or fragment thereof, nucleic acids encoding the p27(Kip1) and FKBP-12 proteins or derivative, analogs or fragments therof, or anti-p27(Kip1):FKBP-12 complex antibodies, or other modulators of p27(Kip1):FKBP-12 complex activity or formation can be tested for activity in treating or preventing autoimmune disease in in vitro and in vivo assays.

In one embodiment of the present invention, a Therapeutic of the present invention can be assayed for activity in treating or preventing autoimmune disease by contacting a cultured cell that exhibits an indicator of an autoimmune reaction in vitro with the Therapeutic, and comparing the level of said indicator in the cell contacted with the Therapeutic with said level of the indicator in a cell not so contacted, wherein a lower level in said contacted cell indicates that the Therapeutic has activity in treating or preventing autoimmune disease. Cell models that can be used for such assays include, but are not limited to, leukocyte and other synovial cells that secrete chemokines mediating inflammation (Kunkel et al., 1996, J. Leukoc. Biol. 59:6-12); cerebrospinal fluid cells from animal models of multiple sclerosis (Norga et al., 1995, Inflamm. Res. 44:529-534); macrophages in experimental autoimmunoneuritis, a model of Guillain-Barre Disease (Bai et al., 1997, J. Neuroimmunol. 76:177-184); CD40/CD40L assays in monocytes (Laman et al., 1996, Crit. Rev. Immunol. 16:59-108); lymphocyte cultures for lpr mice (Nagata, 1996, Prog. Mol. Subcell. Biol. 16:87-103); and cultured thyrocytes in spontaneous murine autoimmune thyroiditis (Green et al., 1996, Endocrinology 137:2823-2832).

In another embodiment of the present invention, a Therapeutic of the present invention can be assayed for activity in treating or preventing autoimmune disease by administering said Therapeutic to a test animal exhibiting an autoimmune reaction or to a test animal that does not exhibit an autoimmune reaction but is subsequently challenged with an agent that elicits an autoimmune reaction, and measuring the change in the autoimmune reaction after the administration of said Therapeutic, wherein a reduction in said autoimmune reaction or a prevention of said autoimmune reaction indicates that the Therapeutic has activity in treating or preventing an autoimmune disease.

A number of animal models of autoimmune disease are known in the art. These models, including those for arthritis, systemic lupus erythematosus, diabetes, thyroiditis, encephalitis etc., accurately mimic natural human autoimmune diseases. Farine, 1997, Toxicol. 119:29-35. Examples of specific models include, but are not limited to, experimental allergic encephalomyelitis for multiple sclerosis (Brabb et al., 1997, J. Immunol. 159:497-507); thyroglobulin-induced experimental thyroiditis (Bhatia et al., 1996 et al., 1996 213:294-300); multiple organ-localized autoimmune disease, e.g., thyroiditis and gastritis in BALB/c nu/nu mice receiving rat thymus grafts under their renal capsules (Taguchi and Takahashi, 1996, Immunology 89:13-19); virus-induced autoimmune diseases such as insulin-dependent diabetes mellitus (Oldstone and von Herath, 1996), experimental autoimmune encephalomyelitis (Encinas et al., 1996, J. Neurosci. Res. 45:655-669); experimental autoimmune labyrinthitis; Freund's-adjuvant induced rheumatoid arthritis and inbred mouse strains that develop systemic lupus erythematosus, rheumatoid arthritis, graft-vs-host disease, and diabetes (Humphryes-Beher, 1996, Adv. Dent. Res. 10:73-75); and autoimmune hepatitis (Meyer zum Buschenfelde and Dienes, 1996, Virchows Arch. 429:1-12).

Similarly, once an organ transplantation disease or disorder has been shown to be amenable to treatment by modulation of p27(Kip1):FKBP-12 complex activity or formation, that disease or disorder can be treated or prevented by administration of a Therapeutic that modulates p27(Kip1):FKBP-12 complex activity or formation. In a specific embodiment, a p27(Kip1):FKBP-12 complex is administered to treat or prevent organ transplantation related diseases or disorders.

p27(Kip1) has been implicated in atherosclerosis as well. The major macrophage colony stimulating factor (MCSF), which is present in atherosclerotic plaques, is required for successful downregulation of p27(Kip1) before cell cycling. Antonov et al., 1997, J. Clin. Invest. 99:2867-2876. A p27(Kip1):FKBP-12 complex or a derivative, analog or fragment thereof, nucleic acids encoding a p27(Kip1) or FKBP-12 protein or a derivative, analog or fragment, or anti-p27(Kip1):FKBP-12 complex antibodies, or other modulators of p27(Kip1):FKBP-12 complex activity or formation, can be tested for activity in treating or preventing atherosclerosis in in vitro and in vivo assays. Accordingly, Therapeutics of the invention, particularly those that modulate (or supply) p27(Kip1):FKBP-12 complex activity or formation, may be effective in treating or preventing atherosclerosis-associated diseases or disorders. Therapeutics of the invention can be assayed by any method known in the art for efficacy in treating or preventing such diseases and disorders.

In one embodiment, a Therapeutic of the present invention can be assayed for activity in treating or preventing atherosclerosis and associated diseases by contacting a cultured cell that exhibits an indicator of an atherosclerosis-associated disease in vitro with the Therapeutic, and comparing the level of said indicator in the cell contacted with the Therapeutic, with said level of the indicator in a cell not so contacted, wherein a lower level in said contacted cell indicates that the Therapeutic has activity in treating or preventing atherosclerosis-associated disease. Specific examples of such cultured models for atherosclerosis and associated diseases include, but are not limited to, monocytes exposed to low density lipoprotein (Frostegard et al., 1996, Atherosclerosis 121:93-103), cloned vascular smooth muscle cells (Suttles et al., 1995, Exp. Cell Res. 218:331-338), endothelial cell-derived chemoattractant exposed T cells (Katz et al., 1994, J. Leukoc. Biol. 55:567-573), cultured human aortic endothelial cells (Farber et al., 1992, Am. J. Physiol. 262:H1088-1085), and foam cell cultures (Libby et al., 1996, Curr. Opin. Lipidol. 7:330-335).

In another embodiment, a Therapeutic of the present invention can be assayed for activity in treating or preventing atherosclerosis-associated diseases by administering the Therapeutic to a test animal that exhibits symptoms of an atherosclerosis-associated disease or that is predisposed to develop symptoms of an atherosclerosis-associated disease; and measuring the change in said symptoms of the atherosclerosis-associated disease after administration of said Therapeutic, wherein a reduction in the severity of the symptoms of the atherosclerosis-associated disease or prevention of the symptoms of the atherosclerosis-associated disease indicates that the Therapeutic has activity in treating or preventing atherosclerosis-associated disease. Such a test animal can be any one of a number of animal models known in the art for atherosclerosis-associated disease. A limited and non-exclusive list of animal models includes knockout mice for premature atherosclerosis (Kurabayashi and Yazaki, 1996, Int. Angiol. 15:187-194), transgenic mouse models of atherosclerosis (Kappel et al., 1994, FASEB J. 8:583-592), antisense oligonucleotide treatment of animal models (Callow, 1995, Curr. Opin. Cardiol. 10:569-576), transgenic rabbit models for atherosclerosis (Taylor, 1997, Ann. N.Y. Acad. Sci 811:146-152), hypercholesterolemic animal models (Rosenfeld, 1996, Diabetes Res. Clin. Pract. 30 Suppl.:1-11), hyperlipidemic mice (Paigen et al., 1994, Curr. Opin. Lipidol. 5:258-264), and inhibition of lipoxygenase in animals (Sigal et al., 1994, Ann. N.Y. Acad. Sci. 714:211-224).

p27(Kip1) has been implicated in membranous nephropathy. A model of membranous nephropathy, which shows aberrant expression of visceral glomerular epithelial cells, demonstrates a marked upregulation of p27(Kip1). Shankland et al., 1997, Kidney Int 52:404-413. A p27(Kip1):FKBP-12 complex or a derivative, analog or fragment thereof, or nucleic acids encoding a p27(Kip1) or FKBP-12 protein or derivative, analog or fragment, or anti-p27(Kip1):FKBP-12 complex antibodies, or other modulators of p27(Kip1):FKBP-12 complex activity or formation can be tested for activity in treating or preventing nephropathy in in vitro and in vivo assays, as described, supra.

5.6. Screening for Antagonists and Agonists of p27(Kip1):FKBP-12 Complex Activity or Formation

p27(Kip1):FKBP-12 complexes and derivatives, fragments and analogs thereof, as well as nucleic acids encoding p27(Kip1) and FKBP-12 and derivatives, fragments and analogs thereof, can be used to screen for a molecule that binds to a p27(Kip1):FKBP-12 complex or a derivative or fragment thereof, as well as to a p27(Kip1) and/or FKBP-12 encoding nucleic acid molecule or a derivative or fragment thereof, which molecule may be used as an agonist or antagonist of p27(Kip1):FKBP-12 complex activity or formation. Accordingly, the present invention is directed to assays for detecting a molecule that specifically binds to p27(Kip1) or FKBP-12 or a fragment or derivative thereof, or to an p27(Kip1) or FKBP-12 encoding nucleic acid molecule, or a fragment or derivative of the nucleic acid molecule. For example, a recombinant cell expressing both p27(Kip1) and FKBP-12 encoding nucleic acid molecules can be used to recombinantly produce the complex or protein used in these assays, or used to screen for a molecule that binds or interferes with p27(Kip1):FKBP-12 complex activity or formation. In preferred embodiments, polypeptide analogs that have superior stabilities but retain the ability to form a p27(Kip1):FKBP-12 complex, (e.g., p27(Kip1) or FKBP-12 modified to be resistant to proteolytic degradation, or modified to be resistant to oxidative degradation), are used to screen for modulators of p27(Kip1) activity or FKBP-12 activity or p27(Kip1):FKBP-12 complex activity or formation. Such resistant molecules can be generated by substitution of amino acids at proteolytic cleavage sites, the use of chemically derivatized amino acids at proteolytic susceptible sites, and replacement of amino acid residues subject to oxidation, i.e., methionine and cysteine.

A molecule (e.g., a putative binding partner or modulator of p27(Kip1):FKBP-12 complex activity or formation) is contacted with a p27(Kip1):FKBP-12 complex, or fragment or derivative thereof, under conditions conducive to binding or modulation, and then a molecule that specifically binds to or modulates the p27(Kip1):FKBP-12 complex is identified. Similar methods can be used to screen for molecules that bind to p27(Kip1) or FKBP-12 encoding nucleic acid molecules or derivatives thereof.

A particular aspect of the invention relates to identifying a molecule that inhibits or promotes formation or degradation of a p27(Kip1):FKBP-12 complex, e.g., using the method described for screening inhibitors using the-modified yeast matrix mating test described in Section 5.6.1., infra, and in International Patent Publication No. WO 97/47763, entitled “Identification and Comparison of Protein-Protein Interactions that Occur in Populations and Identification of Inhibitors of These Interactions” which is incorporated by reference herein in its entirety.

In one embodiment of the invention, a molecule that modulates p27(Kip1) or FKBP-12 activity, or modulates activity or formation of a complex of p27(Kip1) and FKBP-12, is identified by contacting one or more candidate molecules with p27(Kip1) in the presence of FKBP-12 and measuring the amount of complex that forms between p27(Kip1) and FKBP-12; wherein an increase or decrease in the amount of complex that forms relative to the amount that forms in the absence of the candidate molecules indicates that the molecule(s) modulates the activity of p27(Kip1) or FKBP-12 or modulates activity or formation of said complex of p27(Kip1) and FKBP-12. In preferred embodiments, the modulators are identified by administering a candidate molecule to a transgenic non-human animal expressing both p27(Kip1) and FKBP-12 from promoters that are not the native p27(Kip1) or the native FKBP-12 promoters; more preferably where the candidate molecule is also recombinantly expressed in the transgenic non-human animal. Alternatively, the method for identifying such modulators can be carried out in vitro, preferably with purified p27(Kip1), purified FKBP-12, and a purified candidate molecule.

Methods that can be used to carry out the foregoing are commonly known in the art. Agents to be screened can be provided as mixtures of a limited number of specified compounds, or as compound libraries, peptide libraries and the like. Agents to be screened may also include all forms of antisera, antisense nucleic acids, etc., that can modulate p27(Kip1):FKBP-12 complex activity or formation.

By way of example, diversity libraries, such as random or combinatorial peptide or non-peptide libraries, can be screened for molecules that specifically bind to a p27(Kip1):FKBP-12 complex. Many libraries are known in the art and can be used, e.g., chemically synthesized libraries; recombinant, e.g., phage display libraries; and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, BioTechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

In vitro translation-based libraries include but are not limited to those described in PCT Publication No. WO 91/05058 dated Apr. 18, 1991 and in Mattheakis et al., 1994, Proc. Natl. Acad. Sci. USA 91:9022-9026.

By way of examples of non-peptide libraries, a benzodiazepine library (see, e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al., 1994, Proc. Natl. Acad. Sci. USA 91:11138-11142.

Screening the libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and International Patent Publication No. WO 94/18318.

In a specific embodiment, screening can be carried out by contacting the library members with a p27(Kip1):FKBP-12 complex (or encoding nucleic acid molecule or derivative of the complex or molecule) immobilized on a solid phase and harvesting those library members that bind to the complex (or encoding nucleic acid molecule or derivative of the complex or molecule). Examples of such screening methods, termed “panning” techniques, are described by way of example in Parmley and Smith, 1988, Gene 73:305-318; Fowlkes et al., 1992, BioTechniques 13:422-427; International Patent Publication No. WO 94/18318; and in references cited hereinabove.

In a specific embodiment, fragments and/or analogs of p27(Kip1) or FKBP-12, especially peptidomimetics, are screened for activity as competitive or non-competitive inhibitors of p27(Kip1):FKBP-12 complex formation, which thereby inhibit p27(Kip1):FKBP-12 complex activity.

In a preferred embodiment, molecules that bind to a p27(Kip1):FKBP-12 complex can be screened for using the modified yeast mating test described in Section 5.6.1, supra, and exemplified in Section 6.1, infra.

In one embodiment, agents that modulate, i.e., antagonize, or agonize, p27(Kip1):FKBP-12 complex activity or formation can be screened using a binding inhibition assay, wherein agents are screened for their ability to inhibit formation of a p27(Kip1):FKBP-12 complex under aqueous, or physiological, binding conditions in which p27(Kip1):FKBP-12 complex formation occurs in the absence of the agent being tested. Agents that interfere with the formation of p27(Kip1):FKBP-12 complexes are identified as antagonists of complex formation.

Methods for screening may involve labeling the complex proteins with radioligands, e.g., ¹²⁵I or ³H), magnetic ligands, e.g., paramagnetic beads covalently attached to photobiotin acetate), fluorescent ligands, e.g., fluorescein or rhodamine, or enzyme ligands, e.g., luciferase or beta-galactosidase. The reactants that bind in solution can then be isolated by one of many techniques known in the art, including but not restricted to, co-immunoprecipitation of the labeled complex moiety using antisera against the unlabeled binding partner (or labeled binding partner with a distinguishable marker from that used on the labeled complex moiety), immunoaffinity chromatography, size exclusion chromatography, and gradient density centrifugation. In a preferred embodiment, one binding partner is a small fragment or peptidomimetic that is not retained by a commercially available filter. Upon binding, the labeled species is then unable to pass through the filter, providing for a simple assay of complex formation.

Methods commonly known in the art are used to label at least one of the members of the p27(Kip1):FKBP-12 complex. Suitable labeling methods include, but are not limited to, radiolabeling by incorporation of radiolabeled amino acids, e.g., ³H-leucine or ³⁵S-methionine, radiolabeling by post-translational iodination with ¹²⁵I or ¹³¹I using the chloramine T method, Bolton-Hunter reagents, etc., or labeling with ³²P using phosphorylase and inorganic radiolabeled phosphorous, biotin labeling with photobiotin-acetate and sunlamp exposure, etc. In cases where one of the members of the p27(Kip1):FKBP-12 complex is immobilized, the free species is labeled. Where neither of the interacting species is immobilized, each can be labeled with a distinguishable marker such that isolation of both moieties can be followed to provide for more accurate quantitation, and to distinguish the formation of homomeric from heteromeric complexes. Methods that utilize accessory proteins that bind to one of the modified interactants to improve the sensitivity of detection, increase the stability of the complex, etc., are also provided.

Typical binding conditions are, for example, but not by way of limitation, in an aqueous salt solution of 10-250 mM NaCl, 5-50 mM Tris-HCl, pH 5-8, and 0.5% Triton X-100 or other detergent that improves the specificity of the interaction. Metal chelators and/or divalent cations may be added to improve binding and/or reduce proteolysis. Reaction temperatures may include 4, 10, 15, 22, 25, 35, or 42 degrees Celsius, and time of incubation is typically at least 15 seconds, but longer times are preferred to allow binding equilibrium to occur. Particular p27(Kip1):FKBP-12 complexes can be assayed using routine protein binding assays, as described infra, to determine optimal binding conditions for reproducible binding.

The physical parameters of complex formation can be analyzed by quantitation of complex formation using assay methods specific for the label used, e.g., liquid scintillation counting for radioactivity detection, enzyme activity labelled moieties etc. The reaction results are then analyzed utilizing Scatchard analysis, Hill analysis, and other methods commonly known in the arts (e.g., Proteins, Structures, and Molecular Principles, 2^{nd} Edition (1993) Creighton, Ed., W.H. Freeman and Company, New York).

In a second common approach to binding assays, one of the binding species is immobilized on a filter, in a microtiter plate well, in a test tube, to a chromatography matrix, etc., either covalently or non-covalently. Proteins can be covalently immobilized using any method well known in the art, for example, but not limited to the method of Kadonaga and Tjian, 1986, Proc. Natl. Acad. Sci. USA 83:5889-5893, 1986, i.e., linkage.to a cyanogen-bromide derivatized substrate such as CNBr-Sepahrose 4B. Where needed, the use of spacers can reduce steric hindrance from the substrate. Non-covalent attachment of proteins to a substrate include, but are not limited to, attachment of a protein to a charged surface, binding with specific antibodies, binding to a third unrelated interacting protein, etc.

In one embodiment, immobilized p27(Kip1) is used to assay for binding with a radioactively-labeled FKBP-12 in the presence and absence of a compound to be tested for its ability to modulate p27(Kip1):FKBP-12 complex formation. The binding partners are allowed to bind under aqueous, or physiological, conditions, i.e., the conditions under which the original interaction was detected. Conversely, in another embodiment, the FKBP-12 is immobilized and contacted with the labeled p27(Kip1) protein or derivative thereof under binding conditions.

Assays of agents (including cell extracts or a library pool) for competition for binding of one member of a p27(Kip1):FKBP-12 complex, or a derivative thereof, with the other member of the p27(Kip1):FKBP-12 complex labeled by any means, are provided to screen for competitors of p27(Kip1):FKBP-12 complex formation.

In specific embodiments, blocking agents to inhibit non-specific binding of reagents to other protein components, or absorptive losses of reagents to plastics, immobilization matrices, etc., are included in the assay mixture. Blocking agents include, but are not restricted to bovine serum albumin; beta-casein; nonfat dried milk; Denhardt's reagent; Ficoll; polyvinylpyrrolidone; nonionic detergents, e.g., NP40, Triton X-100, Tween 20, Tween 80, etc.; ionic detergents, e.g., SDS, LDS, etc.; polyethylene glycol; etc. Appropriate blocking agent concentrations allow p27(Kip1):FKBP-12 complex formation.

After binding is performed, unbound, labeled protein is removed in the supernatant, and the immobilized protein with any bound, labeled protein is washed extensively. The amount of bound label is then quantitated using standard methods in the art to detect the label as described, supra.

5.6.1. Assays for Protein-Protein Interaction

One aspect of the present invention provides methods for assaying and screening fragments, derivatives and analogs of p27(Kip1)-interacting proteins for binding to p27(Kip1) peptides, and fragments, derivatives, homologs and analogs of p27(Kip1) peptides. Derivatives, analogs and fragments of FKBP-12 that interact with p27(Kip1) or a derivative thereof can be identified by means of a yeast matrix mating test system. Because the interactions are screened for in yeast, the intermolecular protein interactions detected in this system generally occur under physiological conditions that mimic the conditions in mammalian cells. Chien et al., 1991, Proc. Natl. Acad. Sci. USA 88:9578-9581.

Identification of interacting proteins by the improved yeast matrix mating test is based upon the detection of the expression of a reporter gene (“Reporter Gene”), the transcription of which is dependent upon the reconstitution of a transcriptional regulator by the interaction of two proteins, each fused to one half of the transcriptional regulator. The bait (p27(Kip1) or a derivative, homolog or analog thereof) and prey proteins, which are proteins to be tested for ability to interact with the bait, are expressed as fusion proteins to a DNA binding domain, and to a transcriptional regulatory domain, respectively, or vice versa. In various specific embodiments, the prey has a complexity of at least 50, 100, 500, 1,000, 5,000, 10,000, or 50,000; or has a complexity in the range of 25 to 100,000, 100 to 100,000, 50,000 to 100,000, or 100,000 to 500,000. For example, the prey population can be one or more nucleic acid molecules that encodes mutants of FKBP-12, which have been generated by site-directed mutagenesis or another method of making mutations in a nucleotide sequence. Preferably, the prey populations are proteins encoded by DNA, e.g., cDNA or genomic DNA or synthetically generated DNA. For example, the populations can be expressed from chimeric genes comprising cDNA sequences from an un-characterized sample of a population of cDNA from mammalian RNA. Preferably, the prey population are proteins encoded by DNA, e.g., cDNA or genomic DNA or synthetically generated DNA.

In a specific embodiment, recombinant biological libraries expressing random peptides can be used as the source of prey nucleic acids.

Another embodiment of the present invention is directed to a method of screening for inhibitors of the interacting proteins identified herein. Briefly, the protein-protein interaction assay can be carried out as described herein, except that it is done in the presence of one or more candidate molecules. An increase or decrease in Reporter Gene activity relative to that present when the one or more candidate molecules are absent indicates that the candidate molecule has an effect on the interacting pair. In a preferred method, inhibition of the interaction is selected for, i.e., inhibition of the interaction is necessary for the cells to survive, for example, where the interaction activates the URA3 gene, causing yeast to die in medium containing the chemical 5-fluoroorotic acid. Rothstein, 1983, Meth. Enzymol. 101:167-180. The identification of inhibitors of such interactions can also be accomplished, for example, but not by way of limitation, using competitive inhibitor assays, as described supra.

In general, proteins of the bait and prey populations are provided as fusion (chimeric) proteins (preferably by recombinant expression of a chimeric coding sequence) containing each protein contiguous to a pre-selected sequence. For one population, the pre-selected sequence is a DNA binding domain. The DNA binding domain can be any DNA binding domain, as long as it specifically recognizes a DNA sequence within a promoter, or a DNA sequence that modulates the activity of an DNA promoter, e.g., an enhancer element. For example, the DNA binding domain is of a transcriptional activator or inhibitor. For the other population, the pre-selected sequence is an activator or inhibitor domain of a transcriptional activator or inhibitor, respectively. The regulatory domain alone (not as a fusion to a protein sequence) and the DNA-binding domain alone (not as a fusion to a protein sequence) preferably do not detectably interact (so as to avoid false positives in the assay). The assay system further includes a reporter gene operably linked to a promoter that contains a binding site for the DNA binding domain of the transcriptional activator (or inhibitor). Accordingly, in the method of the present invention, binding of a p27(Kip1) fusion protein to a prey fusion protein leads to reconstitution of a transcriptional activator (or inhibitor) which activates (or inhibits) expression of the Reporter Gene. The activation of transcription of the Reporter Gene occurs intracellularly, in prokaryotic or eukaryotic cells, preferably in cell culture.

The promoter that is operably linked to the reporter gene nucleotide sequence can be a native or non-native promoter of the nucleotide sequence, and the DNA binding site(s) that are recognized by the DNA binding domain portion of the fusion protein can be native to the promoter (if the promoter normally contains such binding site(s)) or non-native. Thus, for example, one or more tandem copies (e.g., 4 or 5 copies) of the appropriate DNA binding site can be introduced upstream of the TATA box in the desired promoter (e.g., in the area of position -100 to −400). In a preferred aspect, 4 or 5 tandem copies of the 17 bp UAS (GAL4 DNA binding site) are introduced upstream of the TATA box in the desired promoter, which is upstream of the desired coding sequence for a selectable or detectable marker. In a preferred embodiment, the GAL1-10 promoter is operably fused to the desired nucleotide sequence; the GAL1-10 promoter already contains 5 binding sites for GAL4. Alternatively, the transcriptional activation binding site of the desired gene(s) can be deleted and replaced with GAL4 binding sites. Bartel et al., 1993, BioTechniques 14(6):920-924; Chasman et al., 1989, Mol. Cell. Biol. 9:4746-4749. The Reporter Gene preferably contains a sequence encoding a detectable or selectable marker, the expression of which is regulated by the transcriptional activator, such that the marker is either turned on or off in the cell in response to the presence of a specific interaction. Preferably, the assay is carried out in the absence of background levels of the transcriptional activator, i.e., in a cell that is mutant or otherwise lacking in the transcriptional activator. In one embodiment, more than one Reporter Gene is used to detect transcriptional activation, e.g., one Reporter Gene encoding a detectable marker and one or more Reporter Genes encoding different selectable markers. The detectable marker can be any molecule that can give rise to a detectable signal, e.g., a fluorescent protein or a protein that can be readily visualized or that is recognizable by a specific antibody. The selectable marker can be any protein molecule that confers ability to grow under conditions that do not support the growth of cells not expressing the selectable marker, e.g., the selectable marker is an enzyme that provides an essential nutrient and the cell in which the interaction assay occurs is deficient in the enzyme and the selection medium lacks such nutrient. The Reporter Gene can either be under the control of the native promoter that naturally contains a binding site for the DNA binding protein, or under the control of a heterologous or synthetic promoter.

The activation domain and DNA binding domain used in the assay can be from a wide variety of transcriptional activator proteins, as long as these transcriptional activators have separable binding and transcriptional activation domains. For example, the Gal4 protein of S. cerevisiae, the GCN4 protein of S. cerevisiae (Hope and Struhl, 1986, Cell 46:885-894), the Ard1 protein of S. cerevisiae (Thukral et.al., 1989, Mol. Cell. Biol. 9:2360-2369), the Acel regulatory protein of S. cerevisiae (Thiele et al., 1988, Mol. Cell. Biol. 8:2745-2752), LexA repressor protein of E. coli (Schnarr et al., 1991, Biochimie 73:423-431), herpesvirus VP16 transactivator (Hippenmeyer et al., 1995, Curr. Opin. Biotechnol. 6:548-552), and the human estrogen receptor (Kumar et al., 1987, Cell 51:941-951) each have separable DNA binding and activation domains. The DNA binding domain and activation domain that are employed in the fusion protein need not be from the same transcriptional activator. In a specific embodiment, a Gal4 or LexA DNA binding domain is employed. In another specific embodiment, a Gal4 or herpes simplex virus VP16 (Triezenberg et al., 1988, Genes Dev. 2:730-742) activation domain is employed. In a specific embodiment, amino acids 1-147 of Gal4 (Ma et al., 1987, Cell 48:847-853; Ptashne et al., 1990, Nature 346:329-331) is the DNA binding domain, and amino acids 411-455 of VP16 (Triezenberg et al., 1988, Genes Dev. 2:730-742; Cress et al., 1991, Science 251:87-90) is the activation domain.

In a preferred embodiment, the yeast transcription factor Gal4 is reconstituted by protein-protein interaction and the host strain is mutant for Gal4. In another embodiment, the DNA-binding domain is Ace1 or the activation domain is Ace1, or the DNA binding and activation domains of the Ace1 protein, respectively. Ace1 is a yeast protein that activates transcription from the CUP1 operon in the presence of divalent copper. CUP1 encodes metallothionein, which chelates copper, and the expression of Cup1 protein allows growth in the presence of copper, which is otherwise toxic to the host cells. The Reporter Gene can also be a CUP1-lacZ fusion that expresses the enzyme β-galactosidase (detectable by routine chromogenic assay) upon binding of a reconstituted Ace1 transcriptional activator. Chaudhuri et al., 1995, FEBS Letters 357:221-226. In another specific embodiment, the DNA binding domain of the human estrogen receptor is used, with a Reporter Gene driven by one or three estrogen receptor response elements. Le Douarin et al., 1995, Nucl. Acids. Res. 23:876-878.

The DNA binding domain and the transcription activator/inhibitor domain each preferably has a nuclear localization signal (Ylikomi et al., 1992, EMBO J. 11:3681-3694; Dingwall and Laskey, 1991, TIBS 16:479-481) functional in the cell in which the fusion proteins are to be expressed.

To facilitate isolation of the encoded proteins, he fusion constructs can further contain sequences encoding affinity tags such as glutathione-S-transferase or maltose-binding protein or an epitope of an available antibody, for affinity purification (e.g., binding to glutathione, maltose, or a particular antibody specific for the epitope, respectively. Allen et al., 1995, TIBS 20:511-516. In another embodiment, the fusion constructs further comprise bacterial promoter sequences for recombinant production of the fusion protein in bacterial cells. Allen et al., 1995, TIBS 20:511-516.

The host cell in which the interaction assay occurs can be any cell, prokaryotic or eukaryotic, in which transcription of the Reporter Gene can occur and be detected, including but not limited to mammalian (e.g., monkey, chicken, mouse, rat, human, bovine), bacterial, and insect cells, and is preferably a yeast cell. Expression constructs encoding and capable of expressing the binding domain fusion proteins, the transcriptional activation domain fusion proteins, and the Reporter Gene product(s), are provided within the host cell, by mating of cells containing the expression constructs, or by cell fusion, transformation, electroporation, microinjection, etc. In a specific embodiment in which the assay is carried out in mammalian cells (e.g., hamster cells), the DNA binding domain is the GAL4 DNA binding domain, the activation domain is the herpes simplex virus VP16 transcriptional activation domain, and the Reporter Gene contains the desired reporter gene coding sequence(s) operably linked to a minimal promoter element from the adenovirus E1B gene driven by several GAL4 DNA binding sites. Fearon et al., 1992, Proc. Natl. Acad. Sci. USA 89:7958-7962. The host cell used should not express an endogenous transcription factor that binds to the same DNA site as that recognized by the DNA binding domain fusion population. Also, preferably, the host cell is mutant or otherwise lacking in an endogenous, functional form of the Reporter Gene(s) used in the assay.

Various vectors and host strains for expression of the two fusion protein populations in yeast are known and can be used, see, e.g., Fields et al., U.S. Pat. No. 5,1468,614 dated Nov. 21, 1995; Bartel et al., 1993, “Using the two-hybrid system to detect protein-protein interactions,” in Cellular Interactions in Development, Hartley, D. A. (ed.), Practical Approach Series xviii, IRL Press at Oxford University Press, New York, N.Y., pp. 153-179; Fields and Sternglanz, 1994, Trends in Genetics 10:286-292. By way of example but not limitation, yeast strains or derivative strains made therefrom, which can be used are N105, N106, N1051, N1061, and YULH, as described in Section 6.1, infra. Exemplary strains that can be used in the assay of the invention also include, but are not limited to, the following:

Y190: MATa, ura3-52, his3-200, lys2-801, ade2-101, trp1-901, leu2-3,112, gal4-, gal80-, cyh^{r}2, LYS2: :GAL1_{UAS}-HIS3_{TATA}HIS3, URA³::GAL1_{UAS}-GAL1_{TATA}-lacZ (available from Clontech, Palo Alto, Calif.; Harper et al., 1993, Cell 75:805-816). Y190 contains HIS3 and lacz Reporter Genes driven by GAL4 binding sites.

CG-1945: MATa, ura3-52, his3-200, lys2-801, ade2-101, trp1-901, leu2-3,112, gal4-542, gal80-538, cyh^{r}2, LYS2::GAL1_{UAS}-HIS3_{TATA}HIS3, URA3::GAL1_{UAS17mers(x3)}-CYC1_{TATA}-lacZ (available from Clontech, Palo Alto, Calif.). CG-1945 contains HIS3 and lacZ Reporter Genes driven by GAL4 binding sites.

Y187: MATa, ura3-52, his3-200, ade2-101, trp1-901, leu2-3,112, gal4?, gal80?, URA³::GAL1_{UAS}-GAL1_{TATA}-lacZ (available from Clontech, Palo Alto, Calif.). Y187 contains a lacZ Reporter Gene driven by GAL4 binding sites.

SFY526: MATa, ura3-52, his3-200, lys2-801, ade2-101, trp1-901, leu2-3,112, gal4-542, gal80-538, can^{r}, URA3::GAL1-lacZ (available from Clontech, Palo Alto, Calif.). SFY526 contains HIS3 and lacZ Reporter Genes driven by GAL4 binding sites.

HF7c: MATa, ura3-52, his3-200, lys2-801, ade2-101, trp1-901, leu2-3,112, gal4-542, gal80-538, LYS2::GAL1-HIS3, URA3::GAL1_{UAS 17mers(x3)}-CYC1-lacZ (available from Clontech, Palo Alto, Calif.). HF7c contains HIS3 and lacZ Reporter Genes driven by GAL4 binding sites.

YRG-2: MATa, ura3-52, his3-200, lys2-801, ade2-101, trp1-901, leu2-3,112, gal4-542, gal80-538, LYS2::GAL1_{UAS}-GAL1_{TATA}-HIS³, URA3::GAL1_{UAS17mers(x3)}-CYC1-lacZ (available from Stratagene Cloning Systems, La Jolla, Calif.). YRG-2 contains HIS3 and lacZ Reporter Genes driven by GAL4 binding sites.

Many other strains which are commonly known and available in the art can be used.

If not already lacking in endogenous Reporter Gene activity, cells mutant in the Reporter Gene may be selected by known methods, or the cells can be made mutant in the target Reporter Gene by known gene-disruption methods prior to introducing the Reporter Gene. Rothstein, 1983, Meth. Enzymol. 101:202-211.

In a specific embodiment, plasmids encoding the different fusion protein populations can be both introduced into a single host cell (e.g., a haploid yeast cell) containing one or more Reporter Genes, by co-transformation, to conduct the assay for protein-protein interactions. Or, preferably, the two fusion protein populations are introduced into a single cell either by mating (e.g., of yeast cells) or cell fusions (e.g., of mammalian cells). In a mating type assay, conjugation of haploid yeast cells of opposite mating type that have been transformed with a binding domain fusion expression construct (preferably a plasmid) and an activation (or inhibitor) domain fusion expression construct (preferably a plasmid), respectively, delivers both constructs into the same diploid cell. The mating type of a yeast strain may be manipulated by transformation with the HO gene. Herskowitz and Jensen, 1991, Meth. Enzymol. 194:132-146.

In a preferred embodiment, a yeast interaction mating assay is employed, using two different types of host cells, strain-types a and alpha, of the yeast Saccharomyces cerevisiae. The host cell preferably contains at least two Reporter Genes, each with one or more binding sites for the DNA-binding domain (e.g., of a transcriptional activator). The activator domain and DNA binding domain are each parts of chimeric proteins formed from the two respective populations of proteins. One set of host cells, for example a strain cells, contains fusions of the library of nucleotide sequences with the DNA-binding domain of a transcriptional activator, such as GAL4. The hybrid proteins expressed in this set of host cells are capable of recognizing the DNA-binding site on the Reporter Gene. The second set of yeast host cells, for example alpha strain cells, contains nucleotide sequences encoding fusions of a library of DNA sequences fused to the activation domain of a transcriptional activator. In a preferred embodiment, the fusion protein constructs are introduced into the host cell as a set of plasmids. These plasmids are preferably capable of autonomous replication in a host yeast cell and preferably can also be propagated in E. coli. The plasmid contains a promoter directing the transcription of the DNA binding or activation domain fusion genes, and a transcriptional termination signal. The plasmid also preferably contains a selectable marker gene, permitting selection of cells containing the plasmid. The plasmid can be single-copy or multi-copy. Single-copy yeast plasmids that have the yeast centromere may also be used to express the activation and DNA binding domain fusions. Elledge et al., 1988, Gene 70:303-312. In another embodiment, the fusion constructs are introduced directly into the yeast chromosome via homologous recombination. The homologous recombination for these purposes is mediated through yeast sequences that are not essential for vegetative growth of yeast, e.g., the MER2, MER1, ZIPI, REC102, or ME14 genes.

Bacteriophage vectors can also be used to express the DNA binding domain and/or activation domain fusion proteins. Libraries can generally be prepared faster and more easily from bacteriophage vectors than from plasmid vectors.

A specific embodiment of the present invention is directed to a method of detecting one or more protein-protein interactions comprising (a) recombinantly expressing p27(Kip1) or a derivative, homolog or analog thereof in a first population of yeast cells being of a first mating type and comprising a first fusion protein containing the p27(Kip1) sequence and a DNA binding domain, wherein said first population of yeast cells contains a first nucleotide sequence operably linked to a promoter driven by one or more DNA binding sites recognized by said DNA binding domain such that an interaction of said first fusion protein with a second fusion protein, said second fusion protein comprising a transcriptional activation domain, results in increased transcription of said first nucleotide sequence; (b) negatively selecting to eliminate those yeast cells in said first population in which said increased transcription of said first nucleotide sequence occurs in the absence of said second fusion protein; (c) recombinantly expressing in a second population of yeast cells of a second mating type different from said first mating type, a plurality of said second fusion proteins, each second fusion protein comprising a sequence obtained from a library and an activation domain of a transcriptional activator, in which the activation domain is the same in each said second fusion protein; (d) mating said first population of yeast cells with said second population of yeast cells to form a third population of diploid yeast cells, wherein said third population of diploid yeast cells contains a second nucleotide sequence operably linked to a promoter driven by a DNA binding site recognized by said DNA binding domain such that an interaction of a first fusion protein with a second fusion protein results in increased transcription of said second nucleotide sequence, in which the first and second nucleotide sequences can be the same or different; and (e) detecting said increased transcription of said first and/or second nucleotide sequence, thereby detecting an interaction between a first fusion protein and a second fusion protein.

In a preferred embodiment, the bait p27(Kip1) sequence and the prey library of chimeric genes are combined by mating the two yeast strains on solid media for a period of approximately 6-8 hours. In a less preferred embodiment, the mating is performed in liquid media. The resulting diploids contain both kinds of chimeric genes, i.e., the DNA-binding domain fusion and the activation domain fusion.

Preferred reporter genes include the URA3, HIS3 and/or the lacZ genes (see, e.g., Rose and Botstein, 1983, Meth. Enzymol. 101:167-180) operably linked to GAL4 DNA-binding domain recognition elements. Other reporter genes comprise the functional coding sequences for, but not limited to, Green Fluorescent Protein (GFP) (Cubitt et al., 1995, Trends Biochem. Sci. 20:448-455), luciferase, LEU2, LYS2, ADE2, TRP1, CAN1, CYH2, GUS, CUP1 or chloramphenicol acetyl transferase (CAT). Expression of-LEU2, LYS2, ADE2 and TRP1 are detected by growth in a specific defined media; GUS and CAT can be monitored by well known enzyme assays; and CAN1 and CYH2 are detected by selection in the presence of canavanine and cycloheximide, respectively. With respect to GFP, the natural fluorescence of the protein is detected.

In a specific embodiment, transcription of the Reporter Gene is detected by a linked replication assay. For example, as described by Vasavada et al., 1991, Proc. Natl. Acad. Sci. USA 88:10686-10690, expression of SV40 large T antigen is under the control of the ElB promoter responsive to GAL4 binding sites. The replication of a plasmid containing the SV40 origin of replication, indicates the reconstruction of the GAL4 protein and a protein-protein interaction. Alternatively, a polyoma virus replicon can be employed. Vasavada et al., 1991, Proc. Natl. Acad. Sci. USA 88:10686-10690.

In another embodiment, the expression of Reporter Genes that encode proteins can be detected by immunoassay, i.e., by detecting the immunospecific binding of an antibody to such protein, which antibody can be labeled, or alternatively, which antibody can be incubated with a labeled binding partner to the antibody, so as to yield a detectable signal. Alam and Cook, 1990, Anal. Biochem. 188:245-254 disclose non-limiting examples of detectable marker genes that can be operably linked to a transcriptional regulatory region responsive to a reconstituted transcriptional activator, and thus can be used as Reporter Genes.

The activation of Reporter Genes like URA3 or HIS3 enables the cells to grow in the absence of uracil or histidine, respectively, and hence serves as a selectable marker. Thus, after mating, the cells exhibiting protein-protein interactions are selected for the ability to grow in media lacking a nutritional component, such as uracil or histidine, respectively (referred to as −URA (URA minus) and −HIS (HIS minus) medium, respectively). The −HIS medium preferably contains 3-amino-1,2,4-triazole (3-AT), which is a competitive inhibitor of the HIS3 gene product and thus requires higher levels of transcription to achieve selection. Durfee et al., 1993, Genes Dev. 7:555-569. Similarly, 6-azauracil, which is an inhibitor of the URA3 gene product, can be included in −URA medium. Le Douarin et al., 1995, Nucl. Acids Res. 23:876-878. URA3 gene activity can also be detected and/or measured by determining the activity of its gene product, orotidine-51-monophosphate decarboxylase. Pierrat et al., 1992, Gene 119:237-245; Wolcott et al., 1966, Biochem. Biophys. Acta 122:532-534. In other embodiments of the present invention, the activities of the reporter genes like lacZ or GFP are monitored by measuring a detectable signal (e.g., chromogenic or fluorescent) that results from the activation of these Reporter Genes. For example, lacz transcription can be monitored by incubation in the presence of a chromogenic substrate, such as X-gal (5-bromo-4-chloro-3-indolyl-a-D-galactoside), for its encoded enzyme, β-galactosidase. The pool of all interacting proteins isolated in this manner by mating the p27(Kip1) sequence product and the library identifies the “p27(Kip1) interactive population”.

In a preferred embodiment of the invention, false positives arising from transcriptional activation by the DNA binding domain fusion protein in the absence of a transcriptional activator domain fusion protein are prevented or reduced by negative selection for such activation within a host cell containing the DNA binding fusion population, prior to exposure to the activation domain fusion population. By way of example, if such cell contains URA3 as a Reporter Gene, negative selection is carried out by incubating the cell in the presence of 5-fluoroorotic acid (5-FOA, which kills URA+ cells. Rothstein, 1983, Meth. Enzymol. 101:167-180. Hence, if the DNA-binding domain fusions by themselves activate transcription, the metabolism of 5-FOA will lead to cell death and the removal of self-activating DNA-binding domain hybrids.

Negative selection involving the use of a selectable marker as a Reporter Gene and the presence in the cell medium of an agent toxic or growth inhibitory to the host cells in the absence of Reporter Gene transcription is preferred, since it allows a higher rate of processing than other methods. As will be apparent, negative selection can also be carried out on the activation domain fusion population prior to interaction with the DNA binding domain fusion population, by similar methods, either alone or in addition to negative selection of the DNA binding fusion population.

Negative selection can also be carried out on the recovered p27(Kip1):FKBP-12 pairs by known methods (see, e.g., Bartel et al., 1993, BioTechniques 14:920-924) although pre-negative selection (prior to the interaction assay), as described above, is preferred. For example, each plasmid encoding a protein or peptide or polypeptide fused to the activation domain (one-half of a detected interacting pair) can be transformed back into the original screening strain, either alone or with a plasmid encoding only the DNA-binding domain, the DNA-binding domain fused to the detected interacting protein, or the DNA-binding domain fused to a protein that does not affect transcription or participate in the protein-protein interaction; a positive interaction detected with any plasmid other than that encoding the DNA-binding domain fusion to the detected interacting protein indicates that the activation domain yields false positives, and it is subsequently eliminated from the screen.

In a preferred embodiment, the p27(Kip1) plasmid population is transformed in a yeast strain of a first mating type (a or alpha), and the second plasmid population (containing the library of DNA sequences) is transformed in a yeast strain of different mating type. Both strains are preferably mutant for ura3 and his3, and contain ura3, and optionally lacZ, as Reporter Genes. The first set of yeast cells are positively selected for the p27(Kip1) plasmids and are negatively selected for false positives by incubation in medium lacking the selectable marker (e.g., tryptophan) and containing 5-FOA. Yeast cells of the second mating type are transformed with the second plasmid population, and are positively selected for the presence of the plasmids containing the library of fusion proteins. Selected cells are pooled. Both groups of pooled cells are mixed together and mating is allowed to occur on a solid phase. The resulting diploid cells are then transferred to selective media that selects for the presence of each plasmid and for activation of Reporter Genes.

In a preferred embodiment of the invention, after an interactive population is obtained, the DNA sequences encoding the pairs of interactive proteins are isolated by a method wherein either the DNA-binding domain hybrids or the activation domain hybrids are amplified, in separate respective reactions. Preferably, the amplification is carried out by polymerase chain reaction (PCR) (U.S. Pat. Nos. 4,683,202, 4,683,195 and 4,889,818; Gyllenstein et al., 1988, Proc. Natl. Acad. Sci. USA 85:7652-7656; Ochman et al., 1988, Genetics 120:621-623; Loh et al., 1989, Science 243:217-220; Innis et al., 1990, PCR Protocols, Academic Press, Inc., San Diego, Calif.), using pairs of oligonucleotide primers specific for either the DNA-binding domain hybrids or the activation domain hybrids. This PCR reaction can also be performed on pooled cells expressing interacting protein pairs, preferably as pooled arrays of interactants. Other amplification methods known in the art can be used, including but not limited to ligase chain reaction (EP 320,308), use of Qβ replicase, or methods listed in Kricka et al., 1995, Molecular Probing, Blotting, and Sequencing, Chap. 1 and Table IX, Academic Press, New York.

The plasmids encoding the DNA-binding domain hybrid proteins and the activation domain hybrid proteins can also be isolated and cloned by any of the methods well known in the art. For example, but not by way of limitation, if a shuttle (yeast to E. coli) vector is used to express the fusion proteins, the genes can be recovered by transforming the yeast DNA into E. coli and recovering the plasmids from E. coli (see, e.g., Hoffman et al., 1987, Gene 57:267-272). Alternatively, the yeast vector can be isolated, and the insert encoding the fusion protein subcloned into a bacterial expression vector, for growth of the plasmid in E. coli.

5.7. Pharmaceutical Compositions and Therapeutic/Prophylactic Administration

The present invention provides methods of treatment (and prophylaxis) by administration to a subject in need thereof an effective amount of a Therapeutic of the present invention. In a preferred aspect, the Therapeutic is substantially purified. The subject is preferably an animal, including but not limited to animals such as a cow, pig, horse, chicken, cat, dog, etc., and is preferably a mammal, and most preferably a human. In a specific embodiment, a non-human mammal is the subject.

Formulations and methods of administration that can be employed when the Therapeutic comprises a nucleic acid are described in Sections 5.4.8 and 5.4.9, supra; additional appropriate formulations and routes of administration can be selected from among those described herein below, as well as those known in the art.

Various delivery systems are known and can be used to administer a Therapeutic of the invention, e.g., encapsulation in liposomes, microparticles, and microcapsules: use of recombinant cells capable of expressing the Therapeutic and receptor-mediated endocytosis (see, e.g., Wu and Wu, 1987, J. Biol. Chem. 262:4429-4432); construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (Langer, 1990, Science 249:1527-1533; Treat et al., 1989, In: Liposomes in the Therapy of Infectious Disease and Cancer, Lopez-Berestein and Fidler. eds., Liss, New York, pp. 353-365; Lopez-Berestein, ibid., pp. 317-327; see, generally ibid.)

In yet another embodiment, the Therapeutic can be delivered via a controlled release system. In one embodiment, a pump may be used. Langer, 1009, Science 249:1527-1533; Sefton, 1987, CRC Crit. Ref. Biomed. Eng. 14:201; Buchwald et al., 1980, Surgery 88:507-516; Saudek et al., 1989, N. Engl. J. Med. 321:574-579. In another embodiment, polymeric materials can be used. See, Medical Applications of Controlled Release, Langer and Wise, eds., CRC Press, Boca Raton, Fla., 1974; Controlled Drug Bioavailability, Drug Product Design and Performance, Smolen and Ball, eds., Wiley, New York, 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863. In yet another embodiment, a controlled release system can be placed in proximity of the therapeutic target, e.g., the brain, thus requiring only a fraction of the systemic dose (see, e.g., Goodson, 1984, In: Medical Applications of Controlled Release, supra, Vol. 2, pp. 115-138). Other controlled release systems are discussed in the review by Langer, 1990, Science 249:1527-1533.

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (U.S. Pat. No. 4,980,286), or by direct injection, or by use of microparticle bombardment (e.g., a gene gun; Biolistic, Dupont), or by coating it with lipids, cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid Therapeutic can be introduced intracellularly and incorporated by homologous recombination within host cell DNA for expression.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. optionally associated with such container(s) can be a notice in the form prescribed by a governmental agency regulating the manufacture, use or sale of pharmaceuticals or biological products, which notice reflects approval by the agency of manufacture, use or sale for human administration.

5.8. Animal Models

The present invention also provides animal models. In one embodiment, animal models for diseases and disorders involving p27(Kip1):FKBP-12 complexes are provided. These include but are not limited to cell proliferative disorders including tumorigenesis and tumor spread, degenerative disorders including neurodegenerative disorders, autoimmune disorders, disorders caused by organ transplantation, cardiovascular diseases, and membranous nephropathy. Such animals can be initially produced by promoting homologous recombination or insertional mutagenesis between p27(Kip1) and FKBP-12 genes in the chromosome and exogenous p27(Kip1) and FKBP-12 genes that have been rendered biologically inactive or deleted (preferably by insertion of a heterologous sequence, e.g., an antibiotic resistance gene). In a preferred aspect, homologous recombination is carried out by transforming embryo-derived stem (ES) cells with a vector containing the insertionally inactivated p27(Kip1) and FKBP-12 gene, such that homologous recombination occurs, followed by injecting the transformed ES cells into a blastocyst, and implanting the blastocyst into a foster mother, followed by the birth of the chimeric animal (“knockout animal”) in which a p27(Kip1) and/or FKBP-12 gene has been inactivated or deleted. Capecchi, 1989, Science 244:1288-1292. The chimeric animal can be bred to produce additional knockout animals. Such animals can be mice, hamsters, sheep, pigs, cattle, etc., and are preferably non-human mammals. In a specific embodiment, a knockout mouse is produced.

Such knockout animals are expected to develop, or be predisposed to developing, diseases or disorders involving, but not restricted to, cell proliferative disorders including cancer and benign hypertrophy, various disorders involving cellular apoptosis and cellular differentiation, autoimmune diseases, etc., and thus can have use as animal models of such diseases and disorders, e.g., to screen for or test molecules (e.g., potential Therapeutics) for the ability to inhibit cell proliferative, neurodegenerative, autoimmune, and other diseases.

In a different embodiment of the invention, transgenic animals that have incorporated and express (or overexpress or mis-express) a functional p27(Kip1) and/or FKBP-12 gene, e.g., by introducing the p27(Kip1) and FKBP-12 genes under the control of a heterologous promoter that either overexpresses the protein or proteins, or expresses them in tissues not normally expressing the complexes or proteins, can have use as animal models of diseases and disorders characterized by elevated levels of p27(Kip1):FKBP-12 complexes. Such animals can be used to screen or test molecules for the ability to treat or prevent the diseases and disorders cited, supra.

In one embodiment, the invention provides a recombinant non-human animal in which both an endogenous p27(Kip1) gene and an endogenous FKBP-12 have been deleted or inactivated by homologous recombination or insertional mutagenesis of said animal or an ancestor thereof. In another embodiment, the invention provides a recombinant non-human animal containing both a p27(Kip1) gene and a FKBP-12 gene in which the p27(Kip1) gene is under the control of a promoter that is not the native p27(Kip1) gene promoter and the FKBP-12 gene is under the control of a promoter that is not the native FKBP-12 gene promoter. In a specific embodiment, the invention provides a recombinant non-human animal containing a transgene comprising a nucleic acid sequence encoding a chimeric protein comprising a fragment of p27(Kip1) of at least 6 amino acids fused via a covalent bond to a fragment of FKBP-12 protein of at least 6 amino acids.

### 6. EXAMPLE

6.1. Identification and Specificity of p27(Kip1):FKBP-12 Interactions

6.1.1. Identification of p27(Kip1)

A modified, improved yeast two hybrid system was used to identify protein interactions for the cell cycle protein cyclin dependent kinase (CDK2). Yeast is a eukaryote, and therefore any intermolecular protein interactions detected in this system are likely to occur under physiological conditions in mammalian cells. Chien et al., 1991, Proc. Natl. Acad. Sci. USA 88:9578-9581. One of the identified isolates (prey) was the known p27(Kip1) nucleic acid sequence (GenBank Accession Number U10906), starting from base 127-597 (FIG. 1, SEQ ID NO:1 and SEQ ID NO:2). The interaction between CDK2 and p27(Kip1) has been described before. Kwon et al., 1996 Biochem. Biophys. Res. Comm. 220: 703-709. The nucleic acid sequence and corresponding amino acid sequence of p27(Kip1) are shown in FIG. 1.

6.1.2. Identification and Specificity of p27 (Kip1):FKBP-12 Interaction

In a matrix-mating assay, p27(Kip1), CDK2, FKBP-12, and other proteins were inserted into complementary (a and alpha) mating types of yeast using methods known in the art. Mating was carried out to express both vector constructs within the same yeast cells, thus allowing interaction to occur. Interaction between the domains led to transcriptional activation of reporter genes containing cis-binding elements for Gal4. The reporter genes encoding the indicator protein β-galactosidase, and metabolic markers for uracil and histidine auxotrophy, were included in specific fashion in one or the other of the yeast strains used in the mating. In this way, yeast were selected for successful mating, expression of both fusion constructs, and expression of p27(Kip1)-interacting proteins and the interaction of both fusion proteins.

The p27(Kip1) cDNA was obtained from a commercial fetal brain cDNA library of 3.5×10⁶ independent isolates (Clontech #HL4029AH, Palo Alto, Calif.). The library was synthesized from Xho l-dTl5 primed fetal brain mRNA (from five male/female 19-22 week fetuses) that was directionally cloned into pACT2, a yeast Gal4 activation domain cloning vector including the LEU2 gene for selection in yeast deficient in leucine biosynthesis.

FKBP-12 was amplified from the Clontech pACT2 library by PCR using the forward primer 5′-GGACTAGGCCGAGGTGGCCATGGGAGTGCAGGTGGAAACCATC-3′ (SEQ ID NO:5) and the reverse primer 5′-GGACTAGGCCTCCTGGGCCTCATTCCAGTTTTAGAAGCTCCAC-3′ (SEQ ID NO:6) by standard techniques (nucleotides in italics refer to FKBP-12 sequences, GenBank Accession No. M80199). The fragment was cloned into the SfiI site of the vector pAS, constructed by introducing an SfiI-containing polylinker into the vector pAS2-1 (Clontech, Palo Alto, Calif.). This vector is a yeast DNA-binding domain cloning vector that contains the TRP1 gene for selection in yeast strains deficient in tryptophan biosynthesis. The FKBP-12 sequence was confirmed by nucleic acid sequencing to confirm that PCR amplification reproduced an accurate copy of the FKBP-12 sequence. This test determined that as predicted, the sequence encoded an interacting domain identical to human FKBP-12.

6.1.3. Test for the Specificity of p27(Kip1):FKBP-12 Interaction

p27(Kip1) was transformed by lithium acetate/polyethylene glycol transformation (Ito et al., 1983, J. Bacteriol. 153:163-168) into the yeast strain N106^{r} (mating type a, ura3, his3, ade2, trp1, leu2, gal4, gal80, cyhr, Lys2:::GAL1_{UAS}-HIS3_{TATA}-HIS³, ura 3::GAL1_{UAS}-GAL_{TATA}-lacZ), while the coding sequences of FKBP-12 were transformed into the yeast strain YULH (mating type alpha, ura3, his3, lys2, trp1, leu2, gal4, gal80, GAL1-URA3). The two transformed populations were then mated using standard methods in the art. Sherman et al., Eds., 1991, Getting Started with Yeast, Vol. 194, Academic Press, New York. Briefly, cells were grown until mid-to-late log phase on media that selected for the presence of the appropriate plasmids. The two mating strains, alpha and a, were then, diluted in YAPD media, filtered onto nitrocellulose membranes, and incubated at 30 degrees Celsius for 6-8 hours. The cells were then transferred to media selective for the desired diploids, i.e., yeast harboring reporter genes for beta-galactosidase, uracil auxotrophy, and histidine auxotrophy, and expression of the vectors encoding the bait and prey. The mating products were plated on SC (synthetic complete) (Sambrook et al., 1989, A Laboratory Manual, 2^{nd} Ed., Cold Spring Harbor Press, New York) media lacking adenine and lysine (to select for successful mating), leucine and tryptophan (to select for expression of genes encoded by both plasmids), and uracil and histidine (to select for protein interactions). This medium is herein referred to as SCS medium, for SC Selective medium.

Selected clones were tested for expression of β-galactosidase to confirm the formation of a p27(Kip1):FKBP-12 complex. Filter-lift β-galactosidase assays were performed as modified from the protocol of Breeden and Nasmyth, 1985, Cold Spring Harbor Quant. Biol. 50:643-650. Colonies were patched onto SCS plates, grown overnight, and replica plated onto nitrocellulose filters. The filters were then assayed for β-galactosidase activity as per Breeden and Nasmyth, 1985, Cold Spring Harbor Quant. Biol. 50:643-650. Colonies that were positive turned a visible blue.

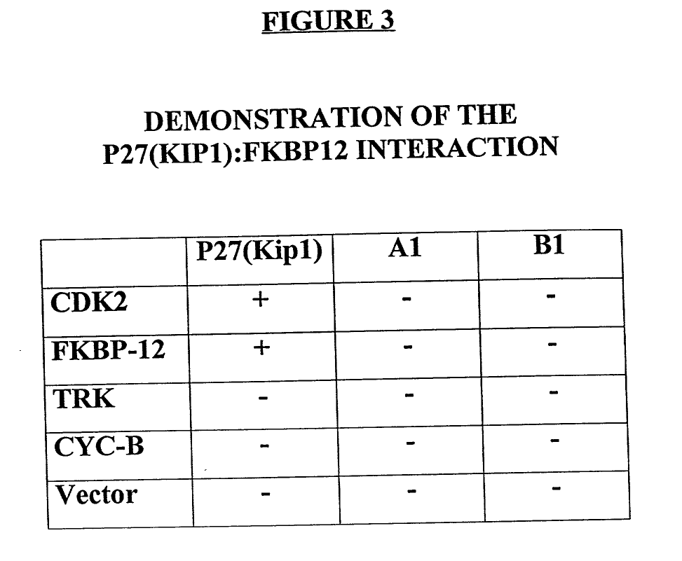

To test for the specificity of p27(Kip1):FKBP-12 interaction, two general tests were first performed. In the first instance, YULH cells expressing FKBP-12 were created and plated on SC (synthetic complete) −Ura plates, grown for 1-2 days, and examined for growth. No growth was found for FKBP-12, confirming that it is not a “self-activating” protein, that is, FKBP-12 requires interaction with a second protein domain for a functional activation complex. In the second instance, plasmids containing p27(Kip1) inserts were transformed into strain N106^{r} (mating type alpha) and mated with yeast strain YULH (mating type a) expressing either CDK2, FKBP-12, or certain other proteins. Promiscuous binders, that is, insert products able to bind with many other proteins in a non-specific fashion, would interact non-specifically with non-CDK2 domains, and would be discarded as non-specific interactants. p27(Kip1) complexed specifically with FKBP-12, but not with trk oncogene (GenBank Accession No. X03541), nor with cyclophilin B (GenBank Accession No. M60857) or the vector-control. As illustrated in FIG. 3, the intersection of the p27(Kip1) column with the FKBP-12 row indicates growth, i.e., a positive interaction. In contrast, the intersection of the p27(Kip1) column with the rows for trk oncogene (trk), cyclophilin B (CYC-B) and vector-control indicates no growth, i.e., no protein interaction. The known interaction between p27(Kip1) and CDK2 was confirmed, as shown in FIG. 3 (intersection of column 1, row 1).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entirety.

What is claimed is:

1. A purified complex of p27(Kip1) and FKBP-12.

2. The purified complex of claim 1 in which the proteins are human proteins.

3. A purified complex selected from the group consisting of a complex of a derivative of p27(Kip1) and FKBP-12, a complex of p27(Kip1) and a derivative of FKBP-12, and a complex of a derivative of p27(Kip1) and a derivative of FKBP-12; in which the derivative of p27(Kip1) is able to form a complex with a wild-type FKBP-12 and the derivative of FKBP-12 is able to form a complex with wild-type p27(Kip1).

4. The purified complex of claim 3 in which the derivative of p27(Kip1) or FKBP-12 is fluorescently labeled.

5. A chimeric protein comprising a fragment of p27(Kip1) consisting of at least 6 amino acids fused via a covalent bond to a fragment of FKBP-12 consisting of at least 6 amino acids.

6. The chimeric protein of claim 5 in which the fragment of p27(Kip1) is a fragment capable of binding FKBP-12 and in which the fragment of FKBP-12 is a fragment capable of binding p27(Kip1).

7. The chimeric protein of claim 6 in which the fragment of p27(Kip1) and the fragment of FKBP-12 form a p27(Kip1):FKBP-12 complex.

8. An antibody which immunospecifically binds the complex of claim 1 or a fragment or derivative of said antibody containing the binding domain thereof.

9. The antibody of claim 8 which does not immunospecifically bind p27(Kip1) or FKBP-12 that is not part of a p27(Kip1):FKBP-12 complex.

10. An isolated nucleic acid or an isolated combination of nucleic acids comprising a nucleotide sequence encoding p27(Kip1) and a nucleotide sequence encoding FKBP-12.

11. The isolated nucleic acid or isolated combination of nucleic acids of claim 10 which are nucleic acid vectors.

12. The isolated nucleic acid or isolated combination of nucleic acids of claim 11 in which the p27(Kip1) coding sequence and the FKBP-12 coding sequence are operably linked to a promoter.

13. An isolated nucleic acid that comprises a nucleotide sequence encoding the chimeric protein of claim 7.

14. A cell containing a nucleic acid of claim 10, which nucleic acid is recombinant.

15. A cell containing a nucleic acid of claim 12, which nucleic acid is recombinant.

16. A recombinant cell containing a nucleic acid of claim 15, which nucleic acid is recombinant.

17. A pharmaceutical composition comprising a therapeutically or prophylactically effective amount of the complex of claim 1; and a pharmaceutically acceptable carrier.

18. The pharmaceutical composition of claim 17 in which the proteins are human proteins.

19. A pharmaceutical composition comprising a therapeutically or prophylactically effective amount of the complex of claim 3; and a pharmaceutically acceptable carrier.

20. A pharmaceutical composition comprising a therapeutically or prophylactically effective amount of the chimeric protein of claim 5; and a pharmaceutically acceptable carrier.

21. A pharmaceutical composition of comprising a therapeutically or prophylactically effective amount of the chimeric protein of claim 6; and a pharmaceutically acceptable carrier.

22. A pharmaceutical composition comprising a therapeutically or prophylactically effective amount of the antibody of claim 8 or a fragment or derivative of said antibody containing the binding domain thereof; and a pharmaceutically acceptable carrier.

23. A pharmaceutical composition comprising a therapeutically or prophylactically effective amount of the antibody of claim 9 or a fragment or derivative of said antibody containing the binding domain thereof; and a pharmaceutically acceptable carrier.

24. A pharmaceutical composition comprising a therapeutically or prophylactically effective amount of the nucleic acid or combination of nucleic acids of claim 10; and a pharmaceutically acceptable carrier.

25. A pharmaceutical composition comprising a therapeutically or prophylactically effective amount of the isolated nucleic acid of claim 13; and a pharmaceutically acceptable carrier.

26. A pharmaceutical composition comprising a therapeutically or prophylactically effective amount of the recombinant cell of claim 14; and a pharmaceutically acceptable carrier.

27. A pharmaceutical composition comprising a therapeutically or prophylactically effective amount of the protein of claim 15; and a pharmaceutically acceptable carrier.

28. A method of producing a complex of p27(Kip1) and FKBP-12 comprising growing a recombinant cell containing the nucleic acid of claim 10 such that the encoded p27(Kip1) and FKBP-12 proteins are expressed and bind to each other, and recovering the expressed complex of p27(Kip1) and FKBP-12.

29. A method of diagnosing or screening for the presence of or a predisposition for developing a disease or disorder characterized by an aberrant level of a complex of p27(Kip1) and FKBP-12, in a subject comprising measuring the level of said complex, RNA encoding p27(Kip1) and FKBP-12, or functional activity of said complex in a sample derived from the subject, in which an increase or decrease in the level of said complex, said RNA encoding p27(Kip1) and FKBP-12, or functional activity of said complex in the sample, relative to the level of said complex, said RNA encoding p27(Kip1) and FKBP-12 or functional activity of said complex found in an analogous sample not having the disease or disorder or a predisposition for developing the disease or disorder, indicates the presence of the disease or disorder or a predisposition for developing the disease or disorder.

30. A kit comprising in one or more containers a substance selected from the group consisting of a complex of p27(Kip1) and FKBP-12, an antibody against said complex, nucleic acid probes capable of hybridizing to RNA of p27(Kip1) and RNA of FKBP-12, or pairs of nucleic acid primers capable of priming amplification of at least a portion of the p27(Kip1) gene and the FKBP-12 gene.

31. A method of treating or preventing a disease or disorder involving aberrant levels of a complex of p27(Kip1) and FKBP-12, in a subject comprising administering to a subject in which such treatment or prevention is desired a therapeutically effective amount of a molecule or molecules that modulate the function of said complex.

32. The method of claim 31 in which said disease or disorder involves decreased levels of said complex and said molecule or molecules promote the function of the complex of p27(Kip1) and FKBP-12 and are selected from the group consisting of a complex of p27(Kip1) and FKBP-12; a derivative or analog of a complex of p27(Kip1) and FKBP-12, which complex is more stable or more active than the wild type complex; nucleic acids encoding p27(Kip1) and FKBP-12 proteins; and nucleic acids encoding a derivative or analog of p27(Kip1) and FKBP-12 that form a complex that is more stable or more active than the wild type complex.

33. The method of claim 31 in which said disease or disorder involves increased levels of said complex and said molecule or molecules inhibit the function of said complex and are selected from the group consisting of an antibody against said complex or a fragment or derivative thereof containing the binding region thereof; p27(Kip1) and FKBP-12 antisense nucleic acids; and nucleic acids comprising at least a portion of the p27(Kip1) and the FKBP—12 gene into which a heterologous nucleotide sequence has been inserted such that said heterologous sequence inactivates the biological activity of the at least a portion of the p27(Kip1) and FKBP-12 genes, in which the p27(Kip1) and the FKBP-12 gene portions flank the heterologous sequences so as to promote homologous recombination with genomic p27(Kip1) and FKBP-12 genes.

34. A method of treating or preventing a disease or disorder involving an aberrant level of FKBP-12 in a subject comprising administering to a subject in which such treatment or prevention is desired a therapeutically effective amount of a molecule that modulates the function of FKBP-12.

35. The method of claim 34 in which said disease or disorder involves a decreased level of FKBP-12 and said molecule promotes the function of FKBP-12 and is selected from the group consisting of the FKBP-12 protein, derivative or analog of FKBP-12 that is active in binding p27(Kip1), a nucleic acid encoding FKBP-12, and a nucleic acid encoding a derivative or analog of FKBP-12 that is active in binding p27(Kip1).

36. The method of claim 34 in which said disease or disorder involves an increased level of FKBP-12 and said molecule inhibits FKBP-12 function and is selected from the group consisting of an anti-FKBP-12 antibody or a fragment or derivative thereof containing the binding region thereof, a FKBP-12 antisense nucleic acid, and a nucleic acid comprising at least a portion of the FKBP-12 gene into which a heterologous nucleotide sequence has been inserted such that said heterologous sequence inactivates the biological activity of the at least a portion of the FKBP-12 gene, in which the FKBP-12 gene portion flanks the heterologous sequence so as to promote homologous recombination with the genomic FKBP-12 gene.

37. A method of treating or preventing a disease or disorder involving an aberrant level of p27(Kip1) in a subject comprising administering to a subject in which such treatment or prevention is desired a therapeutically effective amount of a molecule that modulates the function of p27(Kip1).

38. The method of claim 37 in which said disease or disorder involves a decreased level of p27(Kip1) and said molecule promotes the function of p27(Kip1) and is selected from the group consisting of the p27(Kip1) protein, derivative or analog of p27(Kip1) that is active in binding FKBP-12, a nucleic acid encoding p27(Kip1), and a nucleic acid encoding a derivative or analog of p27(Kip1) that is active in binding FKBP-12.

39. The method of claim 37 in which said disease or disorder involves an increased level of p27(Kip1) and said molecule inhibits p27(Kip1) function and is selected from the group consisting of an anti-p27(Kip1) antibody or a fragment or derivative thereof containing the binding region thereof, a p27(Kip1) antisense nucleic acid, and a nucleic acid comprising at least a portion of the p27(Kip1) gene into which a heterologous nucleotide sequence has been inserted such that said heterologous sequence inactivates the biological activity of the at least a portion of the p27(Kip1) gene, in which the p27(Kip1) gene portion flanks the heterologous sequence so as to promote homologous recombination with the genomic p27(Kip1) gene.

40. A method for screening a purified complex of p27(Kip1) and FKBP-12, or a derivative of said complex, or a modulator of the activity of said complex for activity in treating or preventing atherosclerosis comprising contacting cultured cells that exhibit an indicator of atherosclerosis in vitro with said complex, derivative or modulator; and comparing the level of said indicator in the cells contacted with the complex, derivative, or modulator with said level of said indicator in cells not so contacted, wherein a lower level in said contacted cells indicates that the complex, derivative or modulator has activity in treating or preventing atherosclerosis.

41. A method for screening a purified complex of p27(Kip1) and FKBP-12, or a derivative of said complex, or a modulator of the activity of said complex for activity in treating or preventing an autoimmune disorder comprising contacting cultured cells that exhibit an indicator of a autoimmune disorder in vitro with said complex, derivative or modulator; and comparing the level of said indicator in the cells contacted with the complex, derivative, or modulator with said level of said indicator in cells not so contacted, wherein a lower level in said contacted cells indicates that the complex, derivative or modulator has activity in treating or preventing autoimmune disorder.

42. A method for screening a purified complex of p27(Kip1) and FKBP-12, or a derivative of said complex, or a modulator of the activity of said complex for activity in treating or preventing a neurodegenerative disease comprising contacting cultured cells that exhibit an indicator of a neurodegenerative disease in vitro with said complex, derivative or modulator; and comparing the level of said indicator in the cells contacted with the complex, derivative, or modulator with said level of said indicator in cells not so contacted, wherein a lower level in said contacted cells indicates that the complex, derivative or modulator has activity in treating or preventing neurodegenerative disease.

43. A method of screening a purified complex of p27(Kip1) and FKBP-12, or a derivative of said complex, or a modulator of the activity of said complex for anti-cancer activity comprising measuring the survival or proliferation of cells from a cell line which is derived from or displays characteristics associated with malignant disorder, which cells have been contacted with the complex, derivative, or modulator; and comparing the survival or proliferation in the cells contacted with the complex, derivative or modulator with said survival or proliferation in cells not so contacted, wherein a lower level in said contacted cells indicates that the complex, derivative or modulator has anti-tumor activity.

44. A method of screening a purified complex of p27(Kip1) and FKBP-12, or a derivative of said complex, or a modulator of the activity of said complex for anti-cancer activity by a method comprising administering the complex, derivative or modulator to a test animal, which test animal has a tumor, or which test animal does not have a tumor and is subsequently challenged with tumor cells or tumorigenic agents; and measuring tumor growth or regression in said test animal, wherein decreased tumor growth or increased tumor regression or prevention of tumor growth in test animals administered said complex, derivative or modulator compared to test animals not so administered indicates that the complex, derivative or modulator has anti-cancer activity.

45. A method for screening a purified complex of p27(Kip1) and FKBP-12, or a derivative of said complex, or a modulator of the activity of said complex for activity in treating or preventing membranous nephropathy disorders comprising contacting cultured cells that exhibit an indicator of a membranous nephropathy disorder in vitro with said complex, derivative or modulator; and comparing the level of said indicator in the cells contacted with the complex, derivative, or modulator with said level of said indicator in cells not so contacted, wherein a lower level in said contacted cells indicates that the complex, derivative or modulator has activity in treating or preventing membranous nephropathy disorders.

46. A method for screening a purified complex of p27(Kip1) and FKBP-12, or a derivative of said complex, or a modulator of the activity of said complex for activity in treating or preventing viral infection and associated diseases comprising administering said complex, derivative or modulator to a test animal, which test animal exhibits symptoms of a viral infection or which test animal is predisposed to develop symptoms of a viral infection; and measuring the change in said symptoms of the viral infection after administration of said complex, derivative, or modulator, wherein a reduction in the severity of the symptoms of the viral infection or prevention of the symptoms of the viral infection indicates that the complex, derivative or modulator has activity in treating or preventing viral infection.

47. A method of screening for a molecule that modulates directly or indirectly the formation of a complex of p27(Kip1) and FKBP-12 comprising measuring the levels of said complex formed from p27(Kip1) and FKBP-12 proteins in the presence of said molecule under conditions conducive to formation of the complex; and comparing the levels of said complex with the levels of said complex that are formed in the absence of said molecule, wherein a lower or higher level of said complex in the presence of said molecule indicates that the molecule modulates formation of said complex.

48. A recombinant non-human animal in which both an endogenous p27(Kip1) gene and an endogenous FKBP-12 have been deleted or inactivated by homologous recombination or insertional mutagenesis of said animal or an ancestor thereof.

49. A recombinant non-human animal containing both a p27(Kip1) gene and a FKBP-12 gene, in which the p27(Kip1) gene is under the control of a promoter that is not the native p27(Kip1) gene promoter and the FKBP-12 gene is under the control of a promoter that is not the native FKBP-12 gene promoter.

50. A recombinant non-human animal containing a transgene comprising a nucleic acid sequence encoding the chimeric protein of claim 7.

51. A method of modulating the activity or levels of p27(Kip1) by contacting a cell with, or administering an animal expressing a p27(Kip1) gene, a FKBP-12 protein, or a nucleic acid encoding said protein or an antibody that immunospecifically binds said protein or a fragment or derivative of said antibody containing the binding domain thereof.

52. A method of modulating the activity or levels of FKBP-12 by contacting a cell with, or administering an animal expressing a gene encoding said protein, p27(Kip1), or a nucleic acid encoding p27(Kip1), or an antibody that immunospecifically binds p27(Kip1) or a fragment or derivative of said antibody containing the binding domain thereof.

53. A method of modulating the activity or levels of a complex of p27(Kip1) and FKBP-12 by contacting a cell with, or administering an animal expressing and forming said complex, a molecule that modulates the formation of said complex.

54. A method for identifying a molecule that modulates activity of p27(Kip1) or FKBP-12 or a complex of p27(Kip1) and FKBP-12 comprising contacting one or more candidate molecules with p27(Kip1) in the presence of FKBP-12; and measuring the amount of complex that forms between p27(Kip1) and FKBP-12; wherein an increase or decrease in the amount of complex that forms relative to the amount that forms in the absence of the candidate molecules indicates that the molecules modulate the activity of p27(Kip1) or FKBP-12 or said complex of p27(Kip1) and FKBP-12.

55. The method of claim 54 wherein said contacting is carried out by administering the candidate molecules to the recombinant non-human animal of claim 49.

56. The method of claim 55 wherein said contacting is carried out in vitro; and p27(Kip1), FKBP-12, and said candidate molecules are purified.

57. A method for screening a derivative or analog of p27(Kip1) for biological activity comprising contacting said derivative or analog of p27(Kip1) with FKBP-12; and detecting the formation of a complex between said derivative or analog of p27(Kip1) and FKBP-12; wherein detecting formation of said complex indicates that said derivative or analog of p27(Kip1) has biological activity.

58. A method for screening a derivative or analog of FKBP-12 for biological activity comprising contacting said derivative or analog of FKBP-12 with p27(Kip1); and detecting the formation of a complex between said derivative or analog of FKBP-12 and p27(Kip1); wherein detecting the formation of said complex indicates that said derivative or analog of FKBP-12 has biological activity.

59. A method of monitoring the efficacy of a treatment of a disease or disorder characterized by an aberrant level of a complex of p27(Kip1) and FKBP-12 in a subject administered said treatment for said disease or disorder comprising measuring the level of said complex, RNA encoding p27(Kip1) and FKBP-12, or functional activity of said complex in a sample derived from said subject wherein said sample is taken from said subject after the administration of said treatment and compared to (a) said level in a sample taken from said subject prior to the administration of the treatment or (b) a standard level associated with the pretreatment stage of the disease or disorder, in which the change, or lack of change in the level of said complex, said RNA encoding p27(Kip1) and FKBP-12, or functional activity of said complex in said sample taken after the administration of said treatment relative to the level of said complex, said RNA encoding p27(Kip1) and FKBP-12 or functional activity of said complex in said sample taken before the administration of said treatment or to said standard level indicates whether said administration is effective for treating said disease or disorder.

60. A method of treating or preventing atherosclerosis in a subject comprising administering to a subject in which such treatment or prevention is desired a therapeutically effective amount of a molecule that modulates the function of a complex of p27(Kip1) and FKBP-12.

61. A method of treating or preventing autoimmune disease in a subject comprising administering to a subject in which such treatment or prevention is desired a therapeutically effective amount of a molecule that modulates the function of a complex of p27(Kip1) and FKBP-12.

62. A method of treating or preventing neurodegenerative disease in a subject comprising administering to a.subject in which such treatment or prevention is desired a therapeutically effective amount of a molecule that modulates the function of a complex of p27(Kip1) and FKBP-12.

63. A method of treating or preventing cancer or a hyperproliferative disorder in a subject comprising administering to a subject in which such treatment or prevention is desired a therapeutically effective amount of a molecule that modulates the function of a complex of p27(Kip1) and FKBP-12.

64. A method of treating or preventing membranous nephropathy or an associated disease in a subject comprising administering to a subject in which such treatment or prevention is desired a therapeutically effective amount of a molecule that modulates the function of a complex of p27(Kip1) and FKBP-12.

65. A method of treating or preventing viral infection or an associated disease in a subject comprising administering to a subject in which such treatment or prevention is desired a therapeutically effective amount of a molecule that modulates the function of a complex of p27(Kip1) and FKBP-12.
